(19) United States
(12) Patent Application Publication
(10) Pub. No.: US 2025/0199013 A1
(43) Pub. Date: Jun. 19, 2025

(54) METHODS FOR DETECTING CM-TMA BIOMARKERS

(71) Applicant: Alexion Pharmaceuticals, Inc., Boston, MA (US)

(72) Inventors: Tobin J. Cammett, Southbury, CT (US); Susan Faas McKnight, Old Lyme, CT (US)

(73) Assignee: Alexion Pharmaceuticals, Inc., Boston, MA (US)

(21) Appl. No.: 18/561,670

(22) PCT Filed: May 26, 2022
(86) PCT No.: PCT/US2022/031062

§ 371 Date: Nov. 16, 2023

Related U.S. Application Data
(60) Provisional application No. 63/194,704, filed on May 28, 2021.
(60) Provisional application No. 63/272,033, filed on Oct. 26, 2021.

(51) Int. Cl. G01N 33/68

(52) U.S. Cl. CPC G01N 33/6893; G01N 2333/4716; G01N 2800/226; G01N 2800/52

(57) ABSTRACT

Provided are agents and methods for the detection of complement-mediated thrombotic microangiopathy (CM-TMA) biomarkers. The agents may specifically bind to CM-TMA biomarkers, preferably a proteolytic fragment of complement component factor B (Ba) and soluble C5b9 (sC5b9) and can be used in methods of diagnosis and treatment of CM-TMA, e.g., treatment with an anti-C5 antibody such as ravulizumab (ALXN1210).

### CROSS-REFERENCE TO RELATED PATENT APPLICATIONS

This international patent application claims priority to U.S. Provisional Patent Application No. 63/194,704, filed May 28, 2021, and U.S. Provisional Patent Application No. 63/272,033, filed Oct. 26, 2021 the disclosure of each of which are herein incorporated in its entirety.

### BACKGROUND

### 1. Field

The present disclosure relates to agents and methods for the detection of complement-mediated thrombotic microangiopathy (CM-TMA) biomarkers. The agents may specifically bind to CM-TMA biomarkers, preferably a proteolytic fragment of complement component factor B (Ba) and soluble C5b9 (sC5b9) and can be used in methods of diagnosis and treatment of CM-TMA.

### 2. Description of Related Art

Complement-mediated thrombotic microangiopathy (CM-TMA) is a clinical disorder driven by the generation of excess complement. It is characterized by thrombocytopenia and microangiopathic hemolytic anemia (MAHA) with microvascular thrombosis resulting in systemic organ damage (TMA). One form of CM-TMA, atypical hemolytic uremic syndrome (aHUS), is characterized by pathologic complement activation due to the loss of the natural regulators of the complement system, which results in systemic endothelial and organ damage. Lupus erythematosus is a multisystem immune complex disorder associated with activation of complement, as well as renal failure termed lupus nephritis (LN). A subset of these patients also develop TMA, with progressive life-threatening thrombocytopenia, MAHA, and progressive renal failure similar to aHUS. This subset of patients is poorly responsive to corticosteroids, cyclophosphamide, immunomodulation, and plasma exchange. Park et al. Blood Adv (2018) 2(16): 2090-2094. There exists a need in the art for effective detecting of CM-TMA biomarkers for diagnosis of CM-TMA.

### BRIEF SUMMARY OF THE INVENTION

The disclosure is based, in part, on the identification of biomarkers in urine and/or plasma, which enable diagnosis, prognosis, management, and treatment of patients with CM-TMA. Particularly, the disclosure relates to detection and use of protein biomarkers such as cystatin C; cystatin C/creatinine ratio; complement factor Ba; complement factor Ba/creatinine ratio; complement sC5b-9; and/or complement sC5b-9/creatinine ratio; preferably using a biomarker signature that includes at least two biomarkers Ba and sC5b9, in the diagnosis of CM-TMA and in measurement of patient responsiveness to anti-C5 antibody therapy, such as treatment with ravulizumab (ULTOMIRIS@). In some embodiments, the disclosure additionally relates to use of secondary markers, e.g., estimated glomerular filtration rate (eGFR) and/or urine protein creatinine ratio (UPCR), in addition to the above biomarkers, in CM-TMA diagnosis and/or therapy. Particularly, in embodiments relating to treatment of CM-TMA with anti-C5 antibodies, e.g., ravulizumab (ULTOMIRIS®), it was found that certain biomarkers such as baseline (BL) plasma Ba levels associated with eGFR changes post-treatment. Similarly, baseline (BL) urine sC5b-9, sC5b-9/Cr, urine Ba and Ba/Cr associated with UPCR changes post-treatment.

In an embodiment, a method for assessing complete TMA response may comprise detecting a set of biomarkers in a subject may comprise detecting the level of at least 1, at least 2, at least 3, at least 4, at least 5, at least 6 biomarker(s) selected from the group consisting of cystatin C; cystatin C/creatinine ratio; complement factor Ba; complement factor Ba/creatinine ratio; complement sC5b-9; complement sC5b-9/creatinine ratio, sVCAM-1, sTNF-R1, and/or thrombomodulin; preferably detecting levels of biomarkers in a subset comprising at least two biomarkers comprising Ba and sC5b9, in a fluid biological sample obtained from the subject. The subset of biomarkers may comprise serum sVCAM-1, serum sTNF-R1, plasma thrombomodulin, and urine sC5b-9.

In an embodiment, a method for diagnosing complement-mediated thrombotic microangiopathy (CM-TMA) in a subject may comprise (a) detecting a level of a biomarker selected from the group consisting of cystatin C, cystatin C/creatinine ratio, complement factor Ba, complement factor Ba/creatinine ratio, complement sC5b-9, complement sC5b-9/creatinine ratio, sVCAM-1, sTNF-R1, thrombomodulin, or a combination thereof, in a fluid biological sample obtained from the subject; and (b) comparing the level of the biomarker(s); preferably the levels of the biomarkers in the signature; to a control, wherein an increase in the biomarker levels in the biological sample of the subject compared to that of the control is indicative that the subject suffers from, is suspected of suffering from, or is at risk of suffering from CM-TMA.

In an embodiment, a method for evaluating the treatment of complement-mediated thrombotic microangiopathy (CM-TMA) in a subject may comprise (a) obtaining a fluid biological sample from a subject suffering from complement-mediated thrombotic microangiopathy (CM-TMA); (b) detecting a level of a biomarker selected from the group consisting of cystatin C, cystatin C/creatinine ratio, complement factor Ba, complement factor Ba/creatinine ratio, complement sC5b-9, complement sC5b-9/creatinine ratio, sVCAM-1, sTNF-R1, thrombomodulin, or a combination thereof, in a fluid biological sample obtained from the subject; (c) comparing the level of the biomarker(s) to a control, wherein the control comprises a patient that does not suffer from complement-mediated thrombotic microangiopathy (CM-TMA), (d) treating the subject with complement-mediated thrombotic microangiopathy (CM-TMA); (e) obtaining a fluid biological sample from a subject suffering from complement-mediated thrombotic microangiopathy (CM-TMA); (f) detecting a level of a biomarker selected from the group consisting of cystatin C, cystatin C/creatinine ratio, complement factor Ba, complement factor Ba/creatinine ratio, complement sC5b-9, complement sC5b-9/creatinine ratio, sVCAM-1, sTNF-R1, thrombomodulin, sC5b-9, or a combination thereof, in a fluid biological sample obtained from the subject; (f) comparing the level of the biomarker(s) to a control, wherein the control comprises a patient that does not suffer from complement-mediated thrombotic microangiopathy (CM-TMA).

In an embodiment, a method for detecting biomarkers may comprise (a) obtaining a fluid biological sample from a subject suffering from complement-mediated thrombotic microangiopathy (CM-TMA); and (b) detecting a level of a biomarker selected from the group consisting of cystatin C, cystatin C/creatinine ratio, complement factor Ba, complement factor Ba/creatinine ratio, complement sC5b-9, complement sC5b-9/creatinine ratio, sVCAM-1, sTNF-R1, thrombomodulin, or a combination thereof, in a fluid biological sample obtained from the subject. The method further may comprise comparing the level of the biomarker(s) to a control, wherein the control comprises a patient that does not suffer from complement-mediated thrombotic microangiopathy (CM-TMA).

In an embodiment, the fluid biological sample comprises a heterogeneous sample may comprise plasma Ba, urine Ba/Cr, urine sC5b-9/Cr, and plasma sC5b-9.

In an embodiment, the detection step may comprise detecting serum VCAM-1, serum sTNF-R1, plasma thrombomodulin, and urine sC5b-9.

In an embodiment, a method for diagnosing complement-mediated thrombotic microangiopathy (CM-TMA) in a subject may comprise (a) detecting a level of at least 1, at least 2, at least 3, at least 4, at least 5, at least 6 biomarker(s) selected from the group consisting of cystatin C; cystatin C/creatinine ratio; complement factor Ba; complement factor Ba/creatinine ratio; complement sC5b-9; and/or complement sC5b-9/creatinine ratio; preferably detecting levels of biomarkers in a signature comprising at least two biomarkers comprising Ba and sC5b9, in a fluid biological sample obtained from the subject; and (b) comparing the level of the biomarker(s); preferably the levels of the biomarkers in the signature; to a control, wherein an increase in the biomarker levels in the biological sample of the subject compared to that of the control is indicative that the subject suffers from, is suspected of suffering from, or is at risk of suffering from CM-TMA.

In an embodiment, a method for detecting cystatin C, complement factor Ba, complement sC5b-9 may comprise (a) detecting a level of at least 1, at least 2, at least 3, at least 4, at least 5, at least 6 biomarker(s) selected from the group consisting of cystatin C; cystatin C/creatinine ratio; complement factor Ba; complement factor Ba/creatinine ratio; complement sC5b-9; and/or complement sC5b-9/creatinine ratio; preferably detecting levels of biomarkers in a signature comprising at least two biomarkers comprising Ba and sC5b9, in a fluid biological sample obtained from the subject; and (b) comparing the level of the biomarker(s); preferably the levels of the biomarkers in the signature; to a control, optionally a healthy subject. In an embodiment, an increase in the biomarker levels in the biological sample of the subject compared to that of the control may be indicative that the subject suffers from, is suspected of suffering from, or is at risk of suffering from CM-TMA

In an embodiment, a method for detecting a set of biomarkers in a subject may comprise detecting the biomarkers selected from the group consisting of cystatin C; cystatin C/creatinine ratio; complement factor Ba; complement factor Ba/creatinine ratio; complement sC5b-9; and/or complement sC5b-9/creatinine ratio; preferably detecting levels of biomarkers in a subset comprising at least two biomarkers comprising Ba and sC5b9, in a fluid biological sample obtained from the subject.

In an embodiment, a method for detecting a set of biomarkers in a subject may comprise detecting the biomarkers selected from the group consisting of cystatin C; cystatin C/creatinine ratio; complement factor Ba; complement factor Ba/creatinine ratio; complement sC5b-9, optionally urine sC5b-9; complement sC5b-9/creatinine ratio, sVCAM-1, optionally serum sVACM-1, sTNF-R1, optionally serum sTNF-R1, thrombomodulin, optionally plasma thrombomodulin, or a combination thereof, in a fluid biological sample obtained from the subject.

In an embodiment, the biomarkers may be selected from the group consisting of cystatin C; cystatin C/creatinine ratio; complement factor Ba; complement factor Ba/creatinine ratio; complement sC5b-9, optionally urine sC5b-9; complement sC5b-9/creatinine ratio, sVCAM-1, optionally serum sVACM-1 (Soluble Vascular Cell Adhesion Molecule-1), sTNF-R1 (soluble tumor necrosis factor receptor 1), optionally serum sTNF-R1, thrombomodulin, optionally plasma thrombomodulin, or a combination thereof.

In an embodiment, the fluid biological sample may comprise serum, blood, urine, plasma, or a mixture thereof. The fluid biological sample may comprise urine. The fluid biological sample may comprise plasma. The fluid biological sample may comprise blood. The fluid biological sample may comprise serum. The fluid biological sample may comprise a mixture of urine, plasma, and serum.

In an embodiment, the biomarkers may be associated with the subject's estimated glomerular filtration rate (eGFR) and/or urine protein creatinine ratio (UPCR).

In an embodiment, the method may comprise detecting a level of creatinine in the fluid biological sample from the subject and the control and normalizing the level of the biomarker in the fluid biological samples based on the creatinine level; preferably, wherein the fluid biological sample may comprise urine and the creatinine levels are detected using a creatinine assay.

In an embodiment, the control may comprise an identical biological sample from a healthy subject.

In an embodiment, the biomarker may comprise a protein biomarker selected from cystatin C, complement factor Ba, and/or sC5b9 and the detection comprises contacting the biomarker with an agent comprising an antibody or an antigen-binding fragment thereof that binds, with specificity, to the biomarker. The antibody, or antigen-binding fragment thereof, may be selected from the group consisting of a humanized antibody, a recombinant antibody, a diabody, a chimerized or chimeric antibody, a monoclonal antibody, a deimmunized antibody, a fully human antibody, a single chain antibody, an Fv fragment, an Fd fragment, an Fab fragment, an Fab′ fragment, an F(ab′)₂ fragment, or a combination thereof.

In an embodiment, the detection step may comprise detecting a biomarker signature comprising the following biomarkers: (a) cystatin C+Ba; (b) cystatin C+sC5b9; (c) cystatin C+cystatin C/creatinine; (d) cystatin C+Ba/creatinine; (e) cystatin C+sC5b9/creatinine; (f) Ba+sC5b9; (g) Ba+cystatin C/creatinine; (h) Ba+Ba/creatinine; (i) Ba+sC5b9/creatinine; j) sC5b9+cystatin C/creatinine; (k) sC5b9+Ba/creatinine; (l) sC5b9+sC5b9/creatinine; (m) cystatin C/creatinine+Ba/creatinine; (n) cystatin C/creatinine+sC5b9/creatinine; or (o) Ba/creatinine+sC5b9/creatinine.

In an embodiment, the detection step may comprise detecting a biomarker signature comprising the following biomarkers: (a) cystatin C+Ba+sC5b9; (b) cystatin C+Ba+cystatin C/creatinine; (c) cystatin C+Ba+Ba/creatinine; (d) cystatin C+Ba+sC5b9/creatinine; (e) cystatin C+sC5b9+cystatin C/creatinine; (f) cystatin C+sC5b9+Ba/creatinine; (g) cystatin C+sC5b9+sC5b9/creatinine; (h) cystatin C+cystatin C/creatinine+Ba/creatinine; (i) cystatin C+cystatin C/creatinine+sC5b9/creatinine; (j) cystatin C+Ba/creatinine+sC5b9/creatinine; (k) Ba+sC5b9+cystatin C/creatinine; (l) Ba+sC5b9+Ba/creatinine; (m) Ba+sC5b9+sC5b9/creatinine; (n) Ba+cystatin C/creatinine+Ba/creatinine; (o) Ba+cystatin C/creatinine+sC5b9/creatinine; (p) Ba+Ba/creatinine+sC5b9/creatinine; (q) sC5b9+cystatin C/creatinine+Ba/creatinine; (r) sC5b9+cystatin C/creatinine+sC5b9/creatinine; (s) sC5b9+Ba/creatinine+sC5b9/creatinine; or (t) cystatin C/creatinine+Ba/creatinine+sC5b9/creatinine.

In an embodiment, the detection step may comprise detecting a biomarker signature comprising the following biomarkers: (a) cystatin C+Ba+sC5b9+cystatin C/creatinine; (b) cystatin C+Ba+sC5b9+Ba/creatinine; (c) cystatin C+Ba+sC5b9+sC5b9/creatinine; (d) cystatin C+sC5b9+cystatin C/creatinine+(e) cystatin C+sC5b9+cystatin C/creatinine+sC5b9/creatinine; (f) cystatin C+cystatin C/creatinine+Ba/creatinine+sC5b9/creatinine; (g) Ba+sC5b9+cystatin C/creatinine+Ba/creatinine; (h) Ba+sC5b9+cystatin C/creatinine+sC5b9/creatinine; (i) Ba+cystatin C/creatinine+Ba/creatinine+sC5b9/creatinine; or (j) sC5b9+cystatin C/creatinine+Ba/creatinine+sC5b9/creatinine.

In an embodiment, the detection step may comprise detecting a biomarker signature comprising the following biomarkers: (a) cystatin C+Ba+sC5b9+cystatin C/creatinine+Ba/creatinine; (b) cystatin C+Ba+sC5b9+cystatin C/creatinine+sC5b9/creatinine; (c) cystatin C+sC5b9+cystatin C/creatinine+Ba/creatinine+sC5b9/creatinine; or (d) Ba+sC5b9+cystatin C/creatinine+Ba/creatinine+sC5b9/creatinine.

In an embodiment, the detection step may comprise detecting a biomarker signature comprising the following biomarkers: (a) cystatin C+Ba+sC5b9+cystatin C/creatinine+Ba/creatinine+sC5b9/creatinine.

In an embodiment, the method may further comprise detecting a plasma biomarker selected from Ba and sC5b9.

In an embodiment, the method may further comprise detecting a plasma biomarker signature comprising Ba and sC5b9.

In an embodiment, the fluid biological sample may comprise a heterogeneous sample comprising urine and plasma and the detection step may comprise detecting a biomarker signature comprising at least one urine biomarker and at least one plasma biomarker selected from: (a) urine cystatin C+plasma Ba; (b) urine cystatin C+plasma sC5b9; (c) urine Ba+plasma Ba; (d) urine Ba+plasma sC5b9; (e) urine sC5b9+plasma Ba; (f) urine sC5b9+plasma sC5b9; (g) urine cystatin C/creatinine+plasma Ba; (h) urine cystatin C/creatinine+plasma sC5b9; (i) urine Ba/creatinine+plasma Ba; (j) Ba/creatinine+plasma sC5b9; (k) urine sC5b9/creatinine+plasma Ba; or (l) urine sC5b9/creatinine+plasma sC5b9.

In an embodiment, the fluid biological sample may comprise a heterogeneous sample comprising urine and plasma and the detection step may comprise detecting a biomarker signature comprising at least one urine biomarker and at least two plasma biomarker selected from: (a) urine cystatin C+plasma Ba+plasma sC5b9; (b) urine Ba+plasma Ba+plasma sC5b9; (c) urine sC5b9+plasma Ba+plasma sC5b9; (d) urine cystatin C/creatinine+plasma Ba+plasma sC5b9; (e) urine Ba/creatinine+plasma Ba+plasma sC5b9; or (f) urine sC5b9/creatinine+plasma Ba+plasma sC5b9.

In an embodiment, the fluid biological sample may comprise a heterogeneous sample comprising urine and plasma and the detection step comprises detecting a biomarker signature comprising at least two urine biomarkers and at least one plasma biomarker selected from: (a) urine cystatin C+urine Ba+plasma Ba OR plasma sC5b9; (b) urine cystatin C+urine sC5b9+plasma Ba OR plasma sC5b9; (c) urine cystatin C+urine cystatin C/creatinine+plasma Ba OR plasma sC5b9; (d) urine cystatin C+urine Ba/creatinine+plasma Ba OR plasma sC5b9; (e) urine cystatin C+urine sC5b9/creatinine+plasma Ba OR plasma sC5b9; (f) urine Ba+urine sC5b9+plasma Ba OR plasma sC5b9; (g) urine Ba+urine cystatin C/creatinine+plasma Ba OR plasma sC5b9; (h) urine Ba+urine Ba/creatinine+plasma Ba OR plasma sC5b9; (i) urine Ba+urine sC5b9/creatinine+plasma Ba OR plasma sC5b9; (j) urine sC5b9+urine cystatin C/creatinine+plasma Ba OR plasma sC5b9; (k) urine sC5b9+urine Ba/creatinine+plasma Ba OR plasma sC5b9; (l) urine sC5b9+urine sC5b9/creatinine+plasma Ba OR plasma sC5b9; (m) urine cystatin C/creatinine+urine Ba/creatinine+plasma Ba OR plasma sC5b9; (n) urine cystatin C/creatinine+urine sC5b9/creatinine+plasma Ba OR plasma sC5b9; or (o) urine Ba/creatinine+urine sC5b9/creatinine+plasma Ba OR plasma sC5b9. The biomarkers can comprise plasma complement factor Ba, sC5b-9, thrombomodulin, and D-dimer; serum sTNF-RI and sVCAM-1; urine factor Ba, sC5b-9, and cystatin C.

In an embodiment, the method may further comprise measuring a secondary marker which comprises the subject's estimated glomerular filtration rate (eGFR).

In an embodiment, the method may further comprise measuring a secondary marker which comprises the subject's urine protein/creatinine ratio (UPCR).

In an embodiment, the level of the biomarker and the secondary marker(s) are elevated compared to the control.

In an embodiment, the control may comprise an identical fluid biological sample obtained from a subject without complement-mediated thrombotic microangiopathy (CM-TMA), optionally, a subject with undetectable levels of urine sC5b9.

In an embodiment, the subject may be a mammal. The subject may be a human.

In an embodiment, the subject may have, may be suspected of having, or may be at risk for developing, complement-mediated thrombotic microangiopathy (CM-TMA).

In an embodiment, the subject may have been or may be being treated with an inhibitor of complement. The inhibitor of complement may comprise a complement C5 inhibitor is selected from the group consisting of an antibody, a small molecule, a polypeptide, a polypeptide analog, a peptidomimetic, and an aptamer, or a combination thereof. The complement C5 inhibitor may be selected from the group consisting of recombinant anti-C5 mini-antibody MB12/22, anti-C5 mini-antibody targeted to the endothelium MB12/22-RGD, C5 specific aptamer ARC187, C5 specific aptamer ARC1905 (Avacincaptad pegol), Staphylococcal superantigen-like protein 7 (SSL7), Ornithodoros moubata C inhibitor (OmCl), or a combination thereof. The inhibitor of complement may comprise an anti-C5 antibody or an antigen-binding fragment thereof which (a) bind with specificity to complement C5 and optionally (b) inhibit the cleavage of C5 into fragments C5a and CSb; preferably, wherein the antigen-binding fragment comprises antibody heavy chain complementarity determining regions 1-3 (V_{H}CDR1-3) and antibody light chain complementarity determining regions 1-3 (V_{L}CDR1-3) of the anti-C5 antibody; more preferably an antigen-binding fragment comprising variable heavy (VH) and variable light (VL) chains of the anti-C5 antibody. The, or antigen-binding fragment thereof, may be selected from the group consisting of a humanized antibody, a recombinant antibody, a diabody, a chimerized or chimeric antibody, a monoclonal antibody, a deimmunized antibody, a fully human antibody, a single chain antibody, an Fv fragment, an Fd fragment, an Fab fragment, an Fab′ fragment, an F(ab′)₂ fragment, or a combination thereof. The anti-C5 antibody may comprise eculizumab, ravulizumab, or a combination thereof or a biosimilar thereof.

In an embodiment, the anti-C5 antibody may comprise ravulizumab or a biosimilar thereof; preferably wherein the treatment comprises treatment with ravulizumab comprising a single loading dose on Day 1, followed by regular maintenance dosing beginning on Day 15, based on the subject's weight, wherein (a) for a subject whose weight is ≥40 to <60 kilograms (kg), treatment comprises 2400 milligrams (mg) loading, then 3000 mg maintenance dose every 8 weeks; (b) for a subject whose weight is ≥60 to <100 kg, treatment comprises 2700 mg loading, then 3300 mg maintenance dose every 8 weeks; and (c) for a subject whose weight is ≥100 kg, treatment comprises 3000 mg loading, then 3600 mg maintenance dose every 8 weeks. The anti-C5 antigen-binding fragment may comprise pexelizumab.

In an embodiment, the subject may have been treated with the inhibitor of complement and the treatment has occurred less than one month, optionally less than 31, 30, 29, 28, 27, 26, 25, 24, 23, 22, 21, 20, 19, 18, 17, 16, 15, 14, 13, 12, 11, 10, 9, 8, 7, 6, 5, 4, 3, 2, or 1 day, prior to obtaining the fluid biological sample from the subject.

In an embodiment, the method may further comprise determining whether the subject is at risk of developing complement-mediated thrombotic microangiopathy (CM-TMA).

In an embodiment, the subject may have been treated or may be being treated with a complement inhibitor under a predetermined dosing schedule, the method further comprises determining whether the patient is therapeutically responsive to the complement inhibitor therapy.

In an embodiment, the subject may have or may be at risk of developing complement-mediated thrombotic microangiopathy (CM-TMA) comprising atypical hemolytic uremic syndrome (aHUS); preferably wherein the CM-TMA comprises renal aHUS.

In an embodiment, a method for monitoring responsiveness of a subject to treatment with an inhibitor of complement C5 comprising detecting a level of at least 1, at least 2, at least 3, at least 4, at least 5, at least 6 biomarker(s) selected from the group consisting of cystatin C; cystatin C/creatinine ratio; complement factor Ba; complement factor Ba/creatinine ratio; complement sC5b-9; and/or complement sC5b-9/creatinine ratio; preferably detecting levels of biomarkers in a signature comprising at least two biomarkers comprising Ba and sC5b9, in a fluid biological sample obtained from the subject before and after the treatment and comparing the level of the biomarker(s); preferably the levels of the biomarkers in the signature, in the subject's fluid biological sample before treatment and after the treatment; wherein the subject has, is suspected of having, or is at risk for developing complement-mediated thrombotic microangiopathy (CM-TMA); wherein the subject has been or is being treated with an inhibitor of complement C5; and wherein a reduction in the level of the biomarker(s) in the subject's fluid biological sample after treatment compared to the level thereof prior to treatment with the complement C5 inhibitor indicates that the subject is responsive to the treatment. The method may further comprise measuring a secondary marker which comprises the subject's estimated glomerular filtration rate (eGFR). The method may further comprise measuring a secondary marker which comprises the subject's urine protein/creatinine ratio (UPCR). The level of the biomarker and the secondary marker(s) may be reduced in the subject's fluid biological sample after the treatment.

In an embodiment, a method for treating complement-mediated thrombotic microangiopathy (CM-TMA) using a complement inhibitor in a manner sufficient to induce a physiological change in CM-TMA-associated biomarker proteins, the method may comprise: (a) determining the level or activity of the CM-TMA-associated biomarker proteins in a biological fluid obtained from the subject, wherein the CM-TMA-associated biomarker proteins are selected from the group consisting of: cystatin C; cystatin C/creatinine ratio; complement factor Ba; complement factor Ba/creatinine ratio; complement sC5b-9; and/or complement sC5b-9/creatinine ratio; preferably determining levels or activities of biomarkers in a signature comprising at least two biomarkers comprising Ba and sC5b9; and (b) administering to a subject having, suspected of having, or at risk for developing, CM-TMA, an inhibitor of complement in an amount and with a frequency sufficient to cause a reduction in the levels or activity of the biomarker or the biomarker signature, as compared to the level or activity thereof in a sample of biological fluid of the same type obtained from the subject prior to treatment with the inhibitor. The complement inhibitor may comprise ravulizumab.

In an embodiment, a method for determining whether a patient with complement-mediated thrombotic microangiopathy (CM-TMA) who is treated with a complement inhibitor under a predetermined dosing schedule is in need of a different dosing schedule, the method may comprise (A) determining whether the CM-TMA patient is responsive to treatment with the complement inhibitor under the predetermined dosing schedule, wherein the determining comprises: determining one or both of the concentration and activity of CM-TMA-associated biomarker proteins in a biological fluid obtained from the subject, wherein the CM-TMA-associated biomarker proteins are selected from the group consisting of cystatin C; cystatin C/creatinine ratio; complement factor Ba; complement factor Ba/creatinine ratio; complement sC5b-9; and/or complement sC5b-9/creatinine ratio; preferably determining levels of biomarkers in a signature comprising at least two biomarkers comprising Ba and sC5b9, and wherein: (a) a reduced level or activity, as compared to the concentration in a sample of biological fluid of the same type obtained from the subject prior to treatment with the inhibitor, of the biomarker or the biomarker signature, indicates that the subject is responsive to treatment with the inhibitor; and (B) if the patient is not responsive to treatment with the complement inhibitor, administering the non-responsive patient a different complement inhibitor or the same complement inhibitor at a higher dose or more frequent dosing schedule as compared to the predetermined dosing schedule. The complement inhibitor may comprise ravulizumab. The biomarkers may be selected from the group consisting of cystatin C; cystatin C/creatinine ratio; complement factor Ba; complement factor Ba/creatinine ratio; complement sC5b-9, optionally urine sC5b-9; complement sC5b-9/creatinine ratio, sVCAM-1, optionally serum sVACM-1; sTNF-R1, optionally serum sTNF-R1; thrombomodulin, optionally plasma thrombomodulin; or combinations thereof.

In an embodiment, a kit for the diagnosis of complement-mediated thrombotic microangiopathy (CM-TMA) may comprise an assay plate and a binding agent optionally together with instructions for using the kit, wherein the binding agent is an antibody, or an antigen-binding fragment thereof, which, independently, bind with specificity to a plurality of biological analytes, wherein the analytes are protein biomarkers of CM-TMA, and wherein the protein biomarkers comprise proteolytic fragment of complement component factor B (Ba) and soluble C5b9 (sC5b-9), optionally together with cystatin C. The biomarkers may be selected from the group consisting of cystatin C; cystatin C/creatinine ratio; complement factor Ba; complement factor Ba/creatinine ratio; complement sC5b-9, optionally urine sC5b-9; complement sC5b-9/creatinine ratio, sVCAM-1, optionally serum sVACM-1; sTNF-R1, optionally serum sTNF-R1, thrombomodulin, optionally plasma thrombomodulin; or combinations thereof. The kit may further comprise reagents for creatinine assay. The kit may further comprise reagents for determining urine protein/creatinine ratio (UPCR).

In an embodiment, a composition for the treatment of complement-mediated thrombotic microangiopathy (CM-TMA) in a subject may comprise an effective amount of a complement inhibitor, wherein the complement inhibitor is an anti-C5 antibody or a C5-binding fragment thereof, preferably eculizumab, ravulizumab, or a biosimilar thereof. The effective amount may be an amount sufficient to reduce the level of a biomarker and/or biomarker signature in the subject compared to the level thereof prior to treatment with the complement inhibitor. The biomarker may comprise at least 1, at least 2, at least 3, at least 4, at least 5, at least 6 biomarker(s) selected from the group consisting of cystatin C; cystatin C/creatinine ratio; complement factor Ba; complement factor Ba/creatinine ratio; complement sC5b-9; complement sC5b-9/creatinine ratio, or a combination thereof. The biomarker signature may comprise at least two biomarkers comprising Ba and sC5b9. The levels of the biomarkers and/biomarker signature may be detected in a fluid biological sample obtained from the subject. The fluid biological sample may be urine, plasma, or a combination thereof. The biomarkers may be selected from the group consisting of cystatin C; cystatin C/creatinine ratio; complement factor Ba; complement factor Ba/creatinine ratio; complement sC5b-9, optionally urine sC5b-9; complement sC5b-9/creatinine ratio, sVCAM-1, optionally serum sVACM-1; sTNF-R1, optionally serum sTNF-R1; thrombomodulin, optionally plasma thrombomodulin; or combinations thereof.

In an embodiment, the disclosure relates to use of an effective amount of a complement inhibitor, wherein the complement inhibitor is an anti-C5 antibody or a C5-binding fragment thereof, preferably eculizumab, ravulizumab, or a biosimilar thereof, for the manufacture of a medicament for the treatment of complement-mediated thrombotic microangiopathy (CM-TMA). The effective amount may be an amount sufficient to reduce the level of a biomarker and/or biomarker signature in the subject compared to the level thereof prior to treatment with the complement inhibitor. The biomarker may comprise at least 1, at least 2, at least 3, at least 4, at least 5, at least 6 biomarker(s) selected from the group consisting of cystatin C; cystatin C/creatinine ratio; complement factor Ba; complement factor Ba/creatinine ratio; complement sC5b-9; complement sC5b-9/creatinine ratio, or a combination thereof. The biomarker signature may comprise at least two biomarkers comprising Ba and sC5b9. The levels of the biomarkers and/biomarker signature may be detected in a fluid biological sample obtained from the subject. The fluid biological sample may be urine, serum, plasma, or a combination thereof. The biomarkers may be selected from the group consisting of cystatin C; cystatin C/creatinine ratio; complement factor Ba; complement factor Ba/creatinine ratio; complement sC5b-9, optionally urine sC5b-9; complement sC5b-9/creatinine ratio; sVCAM-1, optionally serum sVACM-1; sTNF-R1, optionally serum sTNF-R1; thrombomodulin, optionally plasma thrombomodulin, or combinations thereof.

### BRIEF DESCRIPTION OF THE DRAWINGS

For a further understanding of the nature, objects, and advantages of the present disclosure, reference should be had to the following detailed description, read in conjunction with the following drawings, wherein like reference numerals denote like elements.

FIG. 1 depicts biomarkers of complement dysregulation and renal damage decrease with Ravulizumab treatment. p-value is derived from a mixed model for repeated measures analysis, with log-transformed biomarker as the dependent variable, and fixed categorical effect of visit and fixed continuous effect of log-transformed baseline value as a covariate, testing the null hypothesis that the mean change from baseline equals zero, versus the alternative hypothesis that the mean change does not equal zero. Dotted lines represent minimum and maximum of normal donor samples. BL, baseline; Cr, creatinine.

FIG. 2 depicts associations between baseline biomarkers and baseline clinical measures. Regression coefficients were derived from simple linear regression analyses, with the log-transformed baseline clinical measure as the dependent variable and the log-transformed baseline biomarker level as the independent variable. For every 2-fold increase in baseline biomarker, the lab value increases (or decreases) by a factor of 2 (raised to the regression coefficient). To calculate percentage increase (or decrease), subtract 1 from this value and multiply by 100; p-values are derived from a two-sided t-test of the null hypothesis that the regression coefficient equals zero. Spearman correlation coefficients are reported. Outlined boxes indicate statistically significant results for biomarkers of complement dysregulation and renal damage. Highlighting/shading indicates very high correlation. Cr, creatinine; eGFR, estimated glomerular filtration rate; LDH, lactate dehydrogenase; UPCR, urine protein/creatinine ratio.

FIG. 3 depicts associations between baseline biomarkers and changes in clinical measures over 26 weeks of treatment. Regression coefficients were derived from simple linear regression analyses, with the log-transformed simple linear regression analysis with the 26-week change from baseline in clinical measure as the dependent variable and the log(2) of the baseline biomarker level as the independent variable. For every 2-fold increase in baseline biomarker, the change in lab value increases (or decreases) by the regression coefficient; p-values are derived from a two-sided t-test of the null hypothesis that the regression coefficient equals zero. Spearman correlation coefficients are reported. Red boxes indicate statistically significant results for complement-specific biomarkers. Yellow highlight indicates very high correlation. Cr, creatinine; eGFR, estimated glomerular filtration rate; LDH, lactate dehydrogenase; UPCR, urine protein/creatinine ratio.

FIG. 4 depicts data showing that baseline biomarker levels were significantly associated with select clinical outcomes at 26 weeks. *Defined as normalization of platelet count, normalization of LDH, and improvement in serum creatinine. CI, confidence interval; Cr, creatinine; LDH, lactate dehydrogenase; TMA, thrombotic microangiopathy.

FIG. 5 depicts Violin plots of biomarkers in blood observed levels over time to 52 weeks. (A) Plasma Ba; (B) Plasma thrombomodulin; (C) Plasma sC5b-9; (D) Plasma D-dimer; (E) Serum sTNF-R1; (F) Serum sVCAM-1. Ravulizumab dose was determined by bodyweight and given at a loading dose at baseline, dose 2 at Day 15, maintenance dose at Day 71 and once every week thereafter. Horizontal lines represent the 25%, median and 75% quartiles. P-values are calculated from a mixed model for repeated measures analysis with biomarker as dependent variable, and fixed categorical effect of visit and fixed continuous effect of baseline value as covariate. The null hypothesis that the mean change from baseline equals zero was tested against the alternative hypothesis that the mean change does not equal zero.

FIG. 6 depicts Violin plots of biomarkers in urine observed levels over time to 52 weeks. (A) Urine cystatin C/creatinine; (B) Urine sC5b-9/creatinine; (C) Urine Ba/creatinine. Ravulizumab dose was determined by bodyweight and given at a loading dose at baseline, dose 2 at Day 15, maintenance dose at Day 71 and once every week thereafter. Horizontal lines represent the 25%, median and 75% quartiles. P-values are calculated from a mixed model for repeated measures analysis with biomarker as dependent variable, and fixed categorical effect of visit and fixed continuous effect of baseline value as covariate. The null hypothesis that the mean change from baseline equals zero was tested against the alternative hypothesis that the mean change does not equal zero.

FIG. 7 depicts Line plot of eGFR and biomarkers (in blood) change from baseline over time by complete TMA response status at 52 Weeks. (A) Plasma Ba, ng/mL (solid circles); (B) Plasma thrombomodulin, ng/mL (solid circles); (C) Plasma sC5b-9, ng/mL (solid circles); (D) Plasma D-dimer, ng/mL (solid circles); (E) Serum sTNF-R1, ng/mL (solid circles); (F) Serum sVCAM-1, ng/mL (solid circles). Change from baseline for blood and urine biomarkers were compared to the clinical measure of eGFR to 52 weeks. Solid lines denote complete TMA responder; dashed lines denote complete TMA non-responder; solid stars denote eGFR (mL/min/1.73 m²) serum. The p-values are presented in each figure for each of the biomarkers.

FIG. 8 depicts Line plot of eGFR and biomarkers (in urine) change from baseline over time by complete TMA response status at 52 Weeks. (A) Urine cystatin C/creatinine, ng/mg creatinine (solid circles); (B) Urine sC5b-9/creatinine, ng/mg creatinine (solid circles); (C) Urine Ba/creatinine, ng/mg creatinine (solid circles). Change from baseline for blood and urine biomarkers were compared to the clinical measure of eGFR to 52 weeks. Solid lines denote complete TMA responder; dashed lines denote complete TMA non-responder; solid stars denote eGFR (mL/min/1.73 m²) serum. The p-values are presented in each figure for each of the biomarkers.

FIG. 9 depicts Logistic regression analysis of complete TMA response at 52 weeks of treatment based on baseline biomarker levels. Odds ratios are derived from a logistic regression analysis with the response variable as the dependent variable and the log of the baseline biomarker level as the independent variable, and represent the increased (or decreased) odds of achieving the efficacy response for every 2-fold increase in baseline biomarker. CI, confidence interval; Cr, creatinine; LDH, lactate dehydrogenase; TMA, thrombotic microangiopathy.

FIG. 10 depicts Receiver operating characteristic (ROC) curves of Ba and sC5b-9 levels in patients versus normal donors. A. Dot plots for individual sC5b-9 and Ba levels in urine and plasma. B. ROC curves for combined biomarkers in urine and plasma. C. Violin plots for combined biomarkers in urine and plasma. D. Summary table for combined biomarkers. TP, true positive; TN, true negative; FP, false positive; FN, false negative.

FIG. 11 depicts receiver operating characteristic (ROC) curves of Ba and sC5b-9 levels in patients versus normal donors A. ROC curves for individual biomarkers in urine and plasma. B. Violin plots for individual biomarkers in urine and plasma.

### DETAILED DESCRIPTION

Before the subject disclosure is further described, it is to be understood that the disclosure is not limited to the particular embodiments of the disclosure described below, as variations of the particular embodiments may be made and still fall within the scope of the appended claims. It is also to be understood that the terminology employed is for the purpose of describing particular embodiments, and is not intended to be limiting. Instead, the scope of the present disclosure will be established by the appended claims.

In this specification and the appended claims, the singular forms “a,” “an,” and “the” include plural reference unless the context clearly dictates otherwise. Unless defined otherwise, all technical and scientific terms used herein have the same meaning as commonly understood to one of ordinary skill in the art to which this disclosure belongs.

“Antibodies” (Abs) and “immunoglobulins” (Igs) are glycoproteins having the same structural characteristics. While antibodies exhibit binding specificity to a specific antigen, immunoglobulins include both antibodies and other antibody-like molecules which lack antigen specificity.

“Native antibodies and immunoglobulins” are usually heterotetrameric glycoproteins of about 150,000 Daltons, composed of two identical light (L) chains and two identical heavy (H) chains. Each light chain is linked to a heavy chain by one covalent disulfide bond, while the number of disulfide linkages varies between the heavy chains of different immunoglobulin isotypes. Each heavy and light chain also has regularly spaced intrachain disulfide bridges. Each heavy chain has at one end a variable domain (VH) followed by a number of constant domains. Each light chain has a variable domain at one end (VL) and a constant domain at its other end; the constant domain of the light chain is aligned with the first constant domain of the heavy chain, and the light chain variable domain is aligned with the variable domain of the heavy chain. Particular amino acid residues are believed to form an interface between the light- and heavy-chain variable domains. Clothia et al. J. Mol. Biol. 186:651 (1985); Novotny and Haber Proc. Natl. Acad. Sci. U.S.A. 82:4592 (1985).

The term “variable” refers broadly to the fact that certain portions of the variable domains differ extensively in sequence among antibodies and are used in the binding and specificity of each particular antibody for its particular antigen. However, the variability is not evenly distributed throughout the variable domains of antibodies. It is concentrated in three segments called complementarity-determining regions (CDRs) or hypervariable regions both in the light-chain and the heavy-chain variable domains. The more highly conserved portions of variable domains are called the framework (FR). The variable domains of native heavy and light chains each comprise four FR regions, largely adopting a j-sheet configuration, connected by three CDRs, which form loops connecting, and in some cases forming part of, the j-sheet structure. The CDRs in each chain are held together in close proximity by the FR regions and, with the CDRs from the other chain, contribute to the formation of the antigen-binding site of antibodies (see Johnson & Wu “Kabat Databse and its applications: 30 years after the first variability plot” Nucleic Acids Research (2000) 28(1): 214-218). The constant domains are not involved directly in binding an antibody to an antigen, but exhibit various effector functions, such as participation of the antibody in antibody-dependent cellular toxicity.

Papain digestion of antibodies produces two identical antigen-binding fragments, called “Fab” fragments, each with a single antigen-binding site, and a residual “Fc” fragment, whose name reflects its ability to crystallize readily. Pepsin treatment yields an F(ab′) 2 fragment that has two antigen-combining sites and is still capable of cross-linking antigen.

“Fv” is the minimum antibody fragment which contains a complete antigen-recognition and binding site. In a two-chain Fv species, this region consists of a dimer of one heavy and one light chain variable domain in tight, non-covalent association. In a single-chain Fv species, one heavy and one light chain variable domain can be covalently linked by a flexible peptide linker such that the light and heavy chains can associate in a “dimeric” structure analogous to that in a two-chain Fv species. It is in this configuration that the three CDRs of each variable domain interact to define an antigen-binding site on the surface of the VH-VL dimer. Collectively, the six CDRs confer antigen-binding specificity to the antibody. However, even a single variable domain (or half of an Fv comprising only three CDRs specific for an antigen) has the ability to recognize and bind antigen, although at a lower affinity than the entire binding site.

The Fab fragment also contains the constant domain of the light chain and the first constant domain (CH1) of the heavy chain. Fab′ fragments differ from Fab fragments by the addition of a few residues at the carboxy terminus of the heavy chain CH1 domain including one or more cysteines from the antibody hinge region. Fab′-SH is the designation herein for Fab′ in which the cysteine residue(s) of the constant domains bear a free thiol group. F(ab′) 2 antibody fragments originally were produced as pairs of Fab′ fragments which have hinge cysteines between them. Other chemical couplings of antibody fragments are also known.

The “light chains” of antibodies (immunoglobulins) from any vertebrate species can be assigned to one of two clearly distinct types, called kappa (κ) and lambda (λ), based on the amino acid sequences of their constant domains.

Depending on the amino acid sequence of the constant domain of their heavy chains, immunoglobulins can be assigned to different classes. There are five major classes of immunoglobulins: IgA, IgD, IgE, IgG, and IgM, and several of these can be further divided into subclasses (isotypes), e.g., IgG 1, IgG2, IgG3, IgG4, IgA1, and IgA2. The heavy-chain constant domains that correspond to the different classes of immunoglobulins are called α, δ, ε, γ, and μ, respectively. The subunit structures and three-dimensional configurations of different classes of immunoglobulins are well known. “Therapeutic Antibody Engineering” (1^{st} Ed.) Strohl & Strohl Woodhead Publishing (2012). The term “antibody” specifically covers monoclonal antibodies, including antibody fragment clones.

“Antibody fragments” comprise a portion of an intact antibody, generally the antigen binding or variable region of the intact antibody. Examples of antibody fragments include Fab, Fab′, F(ab′) 2, and Fv fragments; diabodies; single-chain antibody molecules, including single-chain Fv (scFv) molecules; and multispecific antibodies formed from antibody fragments. “Human Monoclonal Antibodies: Methods and Protocols” (2^{nd} Ed.) Steinitz (Ed.) Humana Press (2019).

“Monoclonal antibody,” as used herein, refers to broadly to an antibody (or antibody fragment) obtained from a population of substantially homogeneous antibodies, e.g., the individual antibodies comprising the population are identical except for possible naturally occurring mutations that may be present in minor amounts. Monoclonal antibodies are highly specific, being directed against a single antigenic site. Furthermore, in contrast to conventional (polyclonal) antibody preparations which typically include different antibodies directed against different determinants (epitopes), each monoclonal antibody is directed against a single determinant on the antigen. In addition to their specificity, the monoclonal antibodies are advantageous in that they are synthesized by the hybridoma culture, uncontaminated by other immunoglobulins. The modifier “monoclonal” indicates the character of the antibody as being obtained from a substantially homogeneous population of antibodies and is not to be construed as requiring production of the antibody by any particular method. For example, the monoclonal antibodies to be used in accordance with the present invention may be made by the hybridoma method first described by Kohler et al, Nature, 256:495 (1975), or may be made by recombinant DNA methods (see, e.g., U.S. Pat. No. 4,816,567). The “monoclonal antibodies” also include clones of antigen-recognition and binding-site containing antibody fragments (Fv clones) isolated from phage antibody libraries using the techniques described in Clackson et al., Nature, 352:624-628 (1991) and Marks et al., J. Mol. Biol., 222:581-597 (1991), for example.

The monoclonal antibodies herein specifically include “chimeric” antibodies (immunoglobulins) in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological activity (U.S. Pat. No. 4,816,567 to Cabilly et al.; Morrison et al., Proc. Natl. Acad. Sci. USA, 81:6851-6855 (1984)); “Antibody Engineering” Volume 2 (2^{nd} Ed.) Kontermann & Dubel. Springer Press (2010).

A “human” antibody (also called a “fully human” antibody) refers broadly to an antibody that includes human framework regions and all of the CDRs from a human immunoglobulin. In one example, the framework and the CDRs are from the same originating human heavy and/or light chain amino acid sequence. However, frameworks from one human antibody can be engineered to include CDRs from a different human antibody.

“Humanized” forms of non-human (e.g., murine) antibodies are chimeric immunoglobulins, immunoglobulin chains or fragments thereof (such as Fv, Fab, Fab′, F(ab′) 2 or other antigen-binding subsequences of antibodies) which contain minimal sequence derived from non-human immunoglobulin. For the most part, humanized antibodies are human immunoglobulins (recipient antibody) in which residues from a complementarity-determining region (CDR) of the recipient are replaced by residues from a CDR of a non-human species (such as mouse, rat or rabbit) or a synthetic sequence (donor antibody), having the desired specificity, affinity, and capacity. In some instances, Fv framework region (FR) residues of the human immunoglobulin are replaced by corresponding non-human residues. Furthermore, humanized antibodies may comprise residues which are found neither in the recipient antibody nor in the imported CDR or framework sequences. These modifications are made to further refine and optimize antibody performance. In some embodiments, all the CDRs are from the donor immunoglobulin in a humanized immunoglobulin. Constant regions need not be present, but if they are, they should be substantially identical to human immunoglobulin constant regions, e.g., at least about 85-90%, such as about 95% or more identical. Hence, all parts of a humanized immunoglobulin, except possibly the CDRs, are substantially identical to corresponding parts of natural human immunoglobulin sequences. A “humanized antibody” is an antibody comprising a humanized light chain and a humanized heavy chain immunoglobulin. A humanized antibody binds to the same antigen as the donor antibody that provides the CDRs. The acceptor framework of a humanized immunoglobulin or antibody may have a limited number of substitutions by amino acids taken from the donor framework. Humanized or other monoclonal antibodies can have additional conservative amino acid substitutions which have substantially no effect on antigen binding or other immunoglobulin functions. Humanized immunoglobulins can be constructed by means of genetic engineering. See e.g., U.S. Pat. No. 5,585,089.

“Single-chain Fv” or “scFv” antibody fragments comprise the VH and VL domains of antibody, wherein these domains are present in a single polypeptide chain. Generally, the scFv polypeptide further comprises a polypeptide linker between the VH and VL domains which enables the scFv to form the desired structure for antigen binding. For a review of scFv see Pluckthun, in The Pharmacology of Monoclonal Antibodies, vol. 113, Rosenburg and Moore eds., Springer-Verlag, New York, pp. 269-315 (1994).

“Diabodies,” as used herein, refers broadly to small antibody fragments with two antigen-binding sites, which fragments comprise a heavy-chain variable domain (VH) connected to a light-chain variable domain (VL) in the same polypeptide chain (VH-VL). By using a linker that is too short to allow pairing between the two domains on the same chain, the domains are forced to pair with the complementary domains of another chain and create two antigen-binding sites. Diabodies are described more fully in, for example, EP 404,097; WO 93/11161; and Hollinger et al., Proc. Natl. Acad. Sci. USA, 90:6444-6448 (1993).

An “isolated” antibody is one which has been identified and separated and/or recovered from a component of its natural environment. Contaminant components of its natural environment are materials which would interfere with diagnostic or therapeutic uses for the antibody, and may include enzymes, hormones, and other proteinaceous or nonproteinaceous solutes. In preferred embodiments, the antibody will be purified (1) to greater than 95% by weight of antibody as determined by the Lowry method, and most preferably more than 99% by weight, (2) to a degree sufficient to obtain at least 15 residues of N-terminal or internal amino acid sequence by use of a spinning cup sequenator, or (3) to homogeneity by SDS-PAGE under reducing or nonreducing conditions using Coomassie blue or, preferably, silver stain. Isolated antibody includes the antibody in situ within recombinant cells since at least one component of the antibody's natural environment will not be present. Ordinarily, however, isolated antibody will be prepared by at least one purification step.

“Binding agent,” as used herein, refers broadly to any naturally occurring, synthetic or genetically engineered agent, such as protein, that binds an antigen (e.g., a biomarker protein). Binding agents can be or be derived from naturally-occurring antibodies. A binding protein or agent can function similarly to an antibody by binding to a specific antigen to form a complex. Binding agents or proteins can include isolated antigen-binding fragments of antibodies.

“Mammal,” as used herein, refers broadly to any and all warm-blooded vertebrate animals of the class Mammalia, characterized by a covering of hair on the skin and, in the female, milk-producing mammary glands for nourishing the young. Mammals include, but are not limited to, humans, domestic and farm animals, and zoo, sports, or pet animals. Examples of mammals include but are not limited to alpacas, armadillos, capybaras, cats, camels, chimpanzees, chinchillas, cattle, dogs, gerbils, goats, gorillas, guinea pigs, hamsters, horses, humans, lemurs, llamas, mice, non-human primates, pigs, rats, sheep, shrews, squirrels, and tapirs. Mammals include but are not limited to bovine, canine, equine, feline, murine, ovine, porcine, primate, and rodent species. Mammal also includes any and all those listed on the Mammal Species of the World maintained by the National Museum of Natural History, Smithsonian Institution in Washington D.C. Similarly, the term “subject” or “patient” includes both human and veterinary subjects and/or patients.

“Normal,” as used herein, refers broadly to an individual or group of individuals who does/do not have a particular disease or condition (e.g., CM-TMA, aHUS) and is also not suspected of having or being at risk for developing the disease or condition. The term “normal” is also used herein to qualify a biological specimen or sample (e.g., a biological fluid) isolated from a normal or healthy individual or subject (or group of such subjects), for example, a “normal control sample” or “normal control biological fluid”.

A subject “at risk for developing aHUS,” as used herein, refers broadly to a subject having one or more (e.g., two, three, four, five, six, seven, or eight or more) risk factors for developing the disorder. Risk factors for aHUS are well known in the art of medicine and include, e.g., a predisposition to develop the condition, e.g., a family history of the condition or a genetic predisposition to develop the condition such as, e.g., one or more mutations in complement Factor H (CFH), membrane cofactor protein (MCP; CD46), C4b-binding protein, complement factor B (CFB), or complement factor I (CFI). See, e.g., Warwicker et al. (1998) Kidney Int 53:836-844; Richards et al. (2001) Am J Hum Genet 68:485-490; Caprioli et al. (2001) Am Soc Nephrol 12:297-307; Neuman et al. (2003) J Med Genet 40:676-681; Richards et al. (2006) Proc Natl Acad Sci USA 100:12966-12971; Fremeaux-Bacchi et al. (2005) J Am Soc Nephrol 17:2017-2025; Esparza-Gordillo et al. (2005) Hum Mol Genet 14:703-712; Goicoechea de Jorge et al. (2007) Proc Natl Acad Sci USA 104(1):240-245; Blom et al. (2008) J Immunol 180(9):6385-91; and Fremeaux-Bacchi et al. (2004) J Medical Genet 41:e84). See also Kavanagh et al. (2006), supra. Risk factors also include, e.g., infection with Streptococcus pneumoniae, pregnancy, cancer, exposure to anti-cancer agents (e.g., quinine, mitomycin C, cisplatin, or bleomycin), exposure to immunotherapeutic agents (e.g., cyclosporine, OKT3, or interferon), exposure to anti-platelet agents (e.g., ticlopidine or clopidogrel), HIV infection, transplantation, autoimmune disease, and combined methylmalonic aciduria and homocystinuria (cblC). See, e.g., Constantinescu et al. (2004) Am J Kidney Dis 43:976-982; George (2003) Curr Opin Hematol 10:339-344; Gottschall et al. (1994) Am J Hematol 47:283-289; Valavaara et al. (1985) Cancer 55:47-50; Miralbell et al. (1996) J Clin Oncol 14:579-585; Dragon-Durey et al. (2005) J Am Soc Nephrol 16:555-63; and Becker et al. (2004) Clin Infect Dis 39:S267-S275. Thus, a human at risk for developing aHUS can be, e.g., one who has a family history of aHUS and/or one who has an HIV infection. From the above it will be clear that subjects “at risk for developing aHUS” are not all the subjects within a species of interest.

A subject “suspected of having aHUS,” as used herein, refers broadly to one having one or more symptoms of the condition. Symptoms of this condition are well-known to those of skill in the art of medicine and include, e.g., severe hypertension, proteinuria, uremia, lethargy/fatigue, irritability, thrombocytopenia, microangiopathic hemolytic anemia, and renal function impairment (e.g., acute renal failure). It will be clear from the foregoing passage that subjects “suspected of having aHUS” are not all the subjects within a species of interest.

As used herein, a “trigger” in the context of CM-TMA, is an event, situation, or condition which causes CM-TMA to occur. In some embodiments, the CM-TMA trigger is an autoimmune condition or event. In some embodiments, trigger is an infection, such as a bacterial infection, viral infection, fungal infection, or parasitic infection. In some embodiments, the trigger is a transplant, e.g., a bone marrow transplant or a solid organ transplant (e.g., selected from kidney, pancreas, liver, heart, and small bowel transplant). In some embodiments, the trigger is one or more drugs. In some embodiments, the trigger is malignant hypertension.

### The Complement System

The complement system acts in conjunction with other immunological systems of the body to defend against intrusion of cellular and viral pathogens. There are at least 25 complement proteins, which are found as a complex collection of plasma proteins and membrane cofactors. The plasma proteins make up about 10% of the globulins in vertebrate serum. Complement components achieve their immune defensive functions by interacting in a series of intricate but precise enzymatic cleavage and membrane binding events. The resulting complement cascade leads to the production of products with opsonic, immunoregulatory, and lytic functions. A concise summary of the biologic activities associated with complement activation is provided, for example, in The Merck Manual, 16^{th} Edition.

The complement cascade progresses via the classical pathway, the alternative pathway, or the lectin pathway. These pathways share many components, and while they differ in their initial steps, they converge and share the same “terminal complement” components (C5 through C9) responsible for the activation and destruction of target cells.

The classical pathway (CP) is typically initiated by antibody recognition of, and binding to, an antigenic site on a target cell. The alternative pathway (AP) can be antibody independent, and can be initiated by certain molecules on pathogen surfaces. Additionally, the lectin pathway is typically initiated with binding of mannose-binding lectin (MBL) to high mannose substrates. These pathways converge at the point where complement component C3 is cleaved by an active protease to yield C3a and C3b. Other pathways activating complement attack can act later in the sequence of events leading to various aspects of complement function. C3a is an anaphylatoxin. C3b binds to bacterial and other cells, as well as to certain viruses and immune complexes, and tags them for removal from the circulation. This opsonic function of C3b is generally considered to be the most important anti-infective action of the complement system. C3b also forms a complex with other components unique to each pathway to form classical or alternative C5 convertase, which cleaves complement component C5 (hereinafter referred to as “C5”) into C5a and C5b.

Cleavage of C5 releases biologically active species such as for example C5a, a potent anaphylatoxin and chemotactic factor, and C5b which through a series of protein interactions leads to the formation of the lytic terminal complement complex, C5b-9. C5a and C5b-9 also have pleiotropic cell activating properties, by amplifying the release of downstream inflammatory factors, such as hydrolytic enzymes, reactive oxygen species, arachidonic acid metabolites and various cytokines.

C5b combines with C6, C7, and C8 to form the C5b-8 complex at the surface of the target cell. Upon binding of several C9 molecules, the membrane attack complex (MAC, C5b-9, terminal complement complex—TCC) is formed. When sufficient numbers of MACs insert into target cell membranes the openings they create (MAC pores) mediate rapid osmotic lysis of the target cells. Lower, non-lytic concentrations of MACs can produce other effects. In particular, membrane insertion of small numbers of the C5b-9 complexes into endothelial cells and platelets can cause deleterious cell activation. In some cases activation may precede cell lysis.

C3a and C5a are activated complement components. These can trigger mast cell degranulation, which releases histamine from basophils and mast cells, and other mediators of inflammation, resulting in smooth muscle contraction, increased vascular permeability, leukocyte activation, and other inflammatory phenomena including cellular proliferation resulting in hypercellularity. C5a also functions as a chemotactic peptide that serves to attract pro-inflammatory granulocytes to the site of complement activation. C5a receptors are found on the surfaces of bronchial and alveolar epithelial cells and bronchial smooth muscle cells. C5a receptors have also been found on eosinophils, mast cells, monocytes, neutrophils, and activated lymphocytes.

Complement-mediated thrombotic microangiopathy (CM-TMA) is a clinical disorder driven by the generation of excess complement. It is characterized by thrombocytopenia and microangiopathic hemolytic anemia (MAHA) with microvascular thrombosis resulting in systemic organ damage (TMA).

aHUS is a genetic, life threatening disease involving chronic complement dysregulation. Patients afflicted with the disease suffer from, among other things, thrombotic microangiopathy (TMA), which can result in stroke and kidney failure. Eculizumab, an antagonist anti-C5 antibody, has been shown to dramatically reduce TMA, normalize platelet levels, and improve renal function of aHUS patients. Yet, even with the clear and robust clinical benefit of complement inhibitor therapy for aHUS patients, some patients still experience elevated levels of several aHUS biomarker proteins in the face of treatment. See, U.S. Pat. No. 9,494,601.

aHUS can be genetic, acquired, or idiopathic. aHUS can be considered genetic when two or more (e.g., three, four, five, or six or more) members of the same family are affected by the disease at least six months apart and exposure to a common triggering agent has been excluded, or when one or more aHUS-associated gene mutations (e.g., one or more mutations in CFH, MCP/CD46, CFB, or CFI) are identified in a subject. For example, a subject can have CFH-associated aHUS, CFB-associated aHUS, CFI-associated aHUS, or MCP-associated aHUS. Up to 30% of genetic aHUS is associated with mutations in CFH, 12% with mutations in MCP, 5-10% with mutations in CFI, and less than 2% with mutations in CFB. Genetic aHUS can be multiplex (e.g., familial; two or more affected family members) or simplex (e.g., a single occurrence in a family). aHUS can be considered acquired when an underlying environmental factor (e.g., a drug, systemic disease, or viral or bacterial agents that do not result in Shiga-like exotoxins) can be identified. aHUS can be considered idiopathic when no trigger (genetic or environmental) is evident.

The methods described herein may comprise identifying the subject as one having, suspected of having, or at risk for developing aHUS. In addition to use of the aHUS biomarker profiling described herein, laboratory tests can be performed to determine whether a human subject has thrombocytopenia, microangiopathic hemolytic anemia, or acute renal insufficiency. Thrombocytopenia can be diagnosed by a medical professional as one or more of: (i) a platelet count that is less than 150,000/mm³ (e.g., less than 60,000/mm³); (ii) a reduction in platelet survival time that is reduced, reflecting enhanced platelet disruption in the circulation; and (iii) giant platelets observed in a peripheral smear, which is consistent with secondary activation of thrombocytopoiesis. Microangiopathic hemolytic anemia can be diagnosed by a medical professional as one or more of: (i) hemoglobin concentrations that are less than 10 mg/dL (e.g., less than 6.5 mg/dL); (ii) increased serum lactate dehydrogenase (LDH) concentrations (>460 U/L); (iii) hyperbilirubinemia, reticulocytosis, circulating free hemoglobin, and low or undetectable haptoglobin concentrations; and (iv) the detection of fragmented red blood cells (schistocytes) with the typical aspect of burr or helmet cells in the peripheral smear together with a negative Coombs test. See, e.g., Kaplan et al. (1992) “Hemolytic Uremic Syndrome and Thrombotic Thrombocytopenic Purpura,” Informa Health Care (ISBN 0824786637) and Zipfel (2005) “Complement and Kidney Disease,” Springer (ISBN 3764371668).

### Detection of CM-TMA Biomarkers

The present disclosure provides for agents and methods for the detection of CM-TMA biomarkers in a biological fluid in patients afflicted with CM-TMA and/or those CM-TMA patients receiving complement inhibitor therapy. aHUS is difficult to diagnose.

Validated biomarkers for diagnosis and monitoring of patients with complement-mediated thrombotic microangiopathy (CM-TMA) are not clinically available; characterization of biomarkers in patients with atypical hemolytic uremic syndrome (aHUS), a form of CM-TMA, may inform diagnosis, treatment decisions and monitoring for patients with CM-TMA.

There is an unmet need for validated biomarkers for the diagnosis, prognosis and monitoring for patients with CM-TMA. The inventors surprisingly found that measurements of complement factor Ba and sC5b-9 demonstrate clinical utility in identifying the CM-TMA/aHUS subset of TMA patients. Indeed, the inventors surprisingly discovered that vascular tissue biomarkers TNF-RI and thrombomodulin, together with factor Ba, demonstrate potential clinical prognostic utility based on their strong association with kidney dysfunction and complete TMA response to ravulizumab treatment.

The methods described herein may comprise the use of at least 1, at least 2, at least 3, at least 4, at least 5, or at least 6 protein biomarkers selected from (1) cystatin C; (2) complement Ba; (3) sC5b9; (4) cystatin C/creatinine; (5) complement Ba/creatinine; (6) sC5b9/creatinine, in diagnosing, monitoring, and treatment of patients with CM-TMA. In instances wherein the biomarkers are derived from urine, the disclosure provides use of (a) cystatin C; (b) Ba; (c) sC5b9; (d) cystatin C/creatinine; (e) Ba/creatinine; (f) sC5b9/creatinine. More specifically, the disclosure relates to use of the aforementioned biomarkers, either solely, or in conjunction with secondary markers such as estimated glomerular filtration rate (eGFR) and/or urine protein creatinine ratio (UPCR), in CM-TMA diagnosis and treatment, e.g., with an inhibitor of complement such as anti-C5 antibody (such as ravulizumab). In particular, it was observed that that post-treatment initiation, baseline (BL) plasma complement factor Ba was associated with eGFR changes following treatment (e.g., at 26 weeks of infusion with ravulizumab). Similarly, urine sC5b-9, sC5b-9/Cr, urine Ba and Ba/Cr were associated with UPCR changes following treatment (e.g., at 26 weeks of infusion with ravulizumab).

The biomarkers detected in the methods described herein may be selected from the group consisting of cystatin C; cystatin C/creatinine ratio; complement factor Ba; complement factor Ba/creatinine ratio; complement sC5b-9, optionally urine sC5b-9; complement sC5b-9/creatinine ratio; sVCAM-1, optionally serum sVACM-1; sTNF-R1, optionally serum sTNF-R1; thrombomodulin, optionally plasma thrombomodulin; or combinations thereof.

The structures (e.g., amino acid sequences) of the CM-TMA biomarkers of the present disclosure are known and accessioned in databases (e.g., GENBANK and/or UNIPROT). For example, human cystatin C has been accessioned under GENBANK No. NP_000090 (date: 12 Sep. 2021) and UNIPROT No. P01034 (date: Jun. 2, 2021). Human complement B, which is cleaved to form Ba and Bb, has been accessioned under GENBANK No. NP_001701 (date: 26 Jul. 2021) and UNIPROT No. P00751 (date: Jun. 2, 2021). Human C5-C9, which form sC5b9 complex, has been accessioned under GENBANK Nos. NP_001726 (C5 protein; date: 3 Oct. 2021); NP_000056 (C6 protein; date: 20 Apr. 2021); NP_000578 (C7 protein; date: 27 Jun. 2021); NP_000553 (C8α protein; date: 30-Jun. 2021), NP_000057 (C8β protein; date: 24 Jun. 2021), and NP_000597 (C8γ protein; date: 14 Feb. 2021); NP_001728 (C9 protein; date: 26 Jun. 2021) and UNIPROT Nos. P01031 (C5 protein); P13671 (C6 protein); P10643 (C7 protein); P07357 (C8α protein), P07358 (C8β protein), and P07360 (C8γ protein); and P02748 (C9 protein), all accessioned Jun. 2, 2021. Human VCAM-1 has been accessioned under GENBANK No. NP_001069 (date: 7 Sep. 2021) and UNIPROT No. P19320 (date: Jun. 2, 2021). Human TNFR-1 has been accessioned under GENBANK No. NP_001056 (date: 17 Oct. 2021) and UNIPROT No. P19438 (date: Jun. 2, 2021). Human thrombomodulin has been accessioned under GENBANK No. NP_000352 (date: 12 Sep. 2021) and UNIPROT No. P07204 (date: Jun. 2, 2021).

The instant disclosure is further based on the surprising discovery that the complement biomarkers a proteolytic fragment of complement component factor B (Ba) (in plasma and urine) and soluble C5b9 (sC5b-9) (in urine) were associated with kidney function in patients with aHUS at baseline and over 26 weeks of treatment with anti-C5 therapy. Such biomarkers show diagnostic potential in CM-TMA and may predict renal response to complement inhibition.

While the disclosure is not bound by any particular theory, the inventors found that monitoring a patient treated with a complement inhibitor (such as an anti-C5 antibody) for a change in concentration of Ba and/or sC5b-9 is useful for diagnosing a patient as having or at risk of developing CM-TMA, including aHUS. Monitoring the status of Ba and/or sC5b-9 can also be useful for determining whether a CM-TMA patient is responding to therapy with a complement inhibitor. Moreover, evaluating the status of Ba and/or sC5b-9 is also useful for identifying a dose, including a threshold dose, of a complement inhibitor, e.g., anti-05 antibody, that by virtue of its effect on the concentration of Ba and/or sC5b-9 biomarker proteins in the human is sufficient to achieve a clinically-meaningful effect on the disease (e.g., sufficient to treat a complement-associated disease, e.g., aHUS).

The inventors identified biomarkers for CM-TMA, namely Ba and sC5b-9. The inventors discovered that an elevated or, in some cases, reduced concentration of certain proteins is associated with the presence of CM-TMA. Similarly, a reduced or elevated concentration (or activity) of certain proteins in a biological fluid obtained from an CM-TMA patient treated with a complement inhibitor indicates that the patient has responded to therapy with the inhibitor. The complement biomarkers Ba (detected in plasma and urine) and sC5b-9 (detected urine) were associated with kidney function in patients with CM-TMA at baseline and over 26 weeks of treatment with anti-C5 therapy. Accordingly, analysis of the concentration and/or activity level of such proteins can be employed to evaluate, among other things, risk for CM-TMA, diagnose CM-TMA, monitor progression or abatement of CM-TMA, and/or monitor treatment response to a complement inhibitor.

### Tissue-Specific Biomarkers and Combinations Thereof in Signatures

### Urine Biomarkers

Urine biomarkers may be used in the diagnosis, monitoring, and therapy of patients with CM-TMA. Urine biomarkers that are useful for this purpose include (a) cystatin C; (b) Ba; (c) sC5b9; (d) cystatin C/creatinine; (e) Ba/creatinine; and (f) sC5b9/creatinine or any combination thereof, for example, comprising, at least 2, at least 3, at least 4, at least 5, or all 6 of the aforementioned biomarkers.

A urine biomarker signature comprising a combination of the above biomarkers may be detected in a sample, and, optionally, be used in the diagnosis, monitoring, and therapy of patients with CM-TMA. Partly due to additive or even synergistic predictive power of biomarker combinations, it may be desirable to employ such signatures in the various embodiments of the present disclosure. It may be even more desirable when different biomarkers are derived from different biological samples, e.g., a signature including urine sC5b9 protein levels and plasma Ba protein levels.

Examples of urine biomarker signatures comprising at least two urine biomarkers include, but are not limited to: (a) cystatin C+Ba; (b) cystatin C+sC5b9; (c) cystatin C+cystatin C/creatinine; (d) cystatin C+Ba/creatinine; (e) cystatin C+sC5b9/creatinine; (f) Ba+sC5b9; (g) Ba+cystatin C/creatinine; (h) Ba+Ba/creatinine; (i) Ba+sC5b9/creatinine; (j) sC5b9+cystatin C/creatinine; (k) sC5b9+Ba/creatinine; (l) sC5b9+sC5b9/creatinine; (m) cystatin C/creatinine+Ba/creatinine; (n) cystatin C/creatinine+sC5b9/creatinine; and (o) Ba/creatinine+sC5b9/creatinine.

Examples of urine biomarker signatures comprising at least three urine biomarkers include but are not limited to: (a) cystatin C+Ba+sC5b9; (b) cystatin C+Ba+cystatin C/creatinine; (c) cystatin C+Ba+Ba/creatinine; (d) cystatin C+Ba+sC5b9/creatinine; (e) cystatin C+sC5b9+cystatin C/creatinine; (f) cystatin C+sC5b9+Ba/creatinine; (g) cystatin C+sC5b9+sC5b9/creatinine; (h) cystatin C+cystatin C/creatinine+Ba/creatinine; (i) cystatin C+cystatin C/creatinine+sC5b9/creatinine; (j) cystatin C+Ba/creatinine+sC5b9/creatinine; (k) Ba+sC5b9+cystatin C/creatinine; (l) Ba+sC5b9+Ba/creatinine; (m) Ba+sC5b9+sC5b9/creatinine; (n) Ba+cystatin C/creatinine+Ba/creatinine; (o) Ba+cystatin C/creatinine+sC5b9/creatinine; (p) Ba+Ba/creatinine+sC5b9/creatinine; (q) sC5b9+cystatin C/creatinine+Ba/creatinine; (r) sC5b9+cystatin C/creatinine+sC5b9/creatinine; (s) sC5b9+Ba/creatinine+sC5b9/creatinine; and (t) cystatin C/creatinine+Ba/creatinine+sC5b9/creatinine.

Examples of urine biomarker signatures comprising at least four urine biomarkers include but are not limited to: (a) cystatin C+Ba+sC5b9+cystatin C/creatinine; (b) cystatin C+Ba+sC5b9+Ba/creatinine; (c) cystatin C+Ba+sC5b9+sC5b9/creatinine; (d) cystatin C+sC5b9+cystatin C/creatinine+(e) cystatin C+sC5b9+cystatin C/creatinine+sC5b9/creatinine; (f) cystatin C+cystatin C/creatinine+Ba/creatinine+sC5b9/creatinine; (g) Ba+sC5b9+cystatin C/creatinine+Ba/creatinine; (h) Ba+sC5b9+cystatin C/creatinine+sC5b9/creatinine; (i) Ba+cystatin C/creatinine+Ba/creatinine+sC5b9/creatinine; or (j) sC5b9+cystatin C/creatinine+Ba/creatinine+sC5b9/creatinine. Examples of urine biomarker signatures comprising at least five urine biomarkers include but are not limited to: (a) cystatin C+Ba+sC5b9+cystatin C/creatinine+Ba/creatinine; (b) cystatin C+Ba+sC5b9+cystatin C/creatinine+sC5b9/creatinine; (c) cystatin C+sC5b9+cystatin C/creatinine+Ba/creatinine+sC5b9/creatinine; (d) Ba+sC5b9+cystatin C/creatinine+Ba/creatinine+sC5b9/creatinine.

Further representative examples of urine biomarker signatures comprising at least six urine biomarkers include but are not limited to: (a) cystatin C+Ba+sC5b9+cystatin C/creatinine+Ba/creatinine+sC5b9/creatinine. Any one or a combination of the urine biomarker signatures comprising a combination of biomarkers described herein may be used in the methods described herein.

### Plasma Biomarkers

Plasma biomarkers may be used in the diagnosis, monitoring, and therapy of patients with CM-TMA. Plasma biomarkers that are useful for this purpose include (a) complement Ba and (b) sC5b9, or any combination thereof, e.g., comprising, at least 2 of the aforementioned plasma biomarkers.

Plasma biomarker signature may comprise a combination of the above plasma biomarkers. For example, a plasma biomarker signature comprising at least two plasma biomarkers may comprise Ba and sC5b9. Further plasma biomarkers comprise plasma thrombomodulin.

Biomarkers from Heterogeneous Samples

Exemplary biomarker signatures comprising biomarkers from heterogeneous samples (e.g., urine and plasma) may be employed in the diagnostic and therapeutic methods described herein. The exemplary biomarker signatures comprising biomarkers from heterogeneous samples (e.g., urine and plasma) may be detected in fluid biological samples.

A two biomarker signature from heterogeneous samples (at least one from urine and one from plasma) may be employed in the diagnostic and therapeutic methods. The exemplary two biomarker signature from heterogeneous samples may be detected in fluid biological samples. Exemplary heterogeneous two biomarker signatures include but are not limited to (a) urine cystatin C+plasma Ba; (b) urine cystatin C+plasma sC5b9; (c) urine Ba+plasma Ba; (d) urine Ba+plasma sC5b9; (e) urine sC5b9+plasma Ba; (f) urine sC5b9+plasma sC5b9; (g) urine cystatin C/creatinine+plasma Ba; (h) urine cystatin C/creatinine+plasma sC5b9; (i) urine Ba/creatinine+plasma Ba; (j) urine Ba/creatinine+plasma sC5b9; (k) urine sC5b9/creatinine+plasma Ba; (l) urine sC5b9/creatinine+plasma sC5b9.

A three biomarker signature from heterogeneous samples (e.g., including at least one from urine and two from plasma) may be employed in the diagnostic and therapeutic methods described herein. The exemplary three biomarker signatures from heterogeneous samples may be detected in fluid biological samples. Examples of heterogeneous three biomarker signatures include but are not limited to (a) urine cystatin C+plasma Ba+plasma sC5b9; (b) urine Ba+plasma Ba+plasma sC5b9; (c) urine sC5b9+plasma Ba+plasma sC5b9; (d) urine cystatin C/creatinine+plasma Ba+plasma sC5b9; (e) urine Ba/creatinine+plasma Ba+plasma sC5b9; (f) urine sC5b9/creatinine+plasma Ba+plasma sC5b9.

A three biomarker signature from heterogeneous samples (e.g., including at least two from urine and one from plasma) may be employed in the diagnostic and therapeutic methods described herein. The exemplary three biomarker signatures from heterogeneous samples may be detected in fluid biological samples. Examples of heterogeneous three biomarker signatures include but are not limited to (a) urine cystatin C+urine Ba+plasma Ba or plasma sC5b9; (b) urine cystatin C+urine sC5b9+plasma Ba or plasma sC5b9; (c) urine cystatin C+urine cystatin C/creatinine+plasma Ba or plasma sC5b9; (d) urine cystatin C+urine Ba/creatinine+plasma Ba or plasma sC5b9; (e) urine cystatin C+urine sC5b9/creatinine+plasma Ba or plasma sC5b9; (f) urine Ba+urine sC5b9+plasma Ba or plasma sC5b9; (g) urine Ba+urine cystatin C/creatinine+plasma Ba or plasma sC5b9; (h) urine Ba+urine Ba/creatinine+plasma Ba or plasma sC5b9; (i) urine Ba+urine sC5b9/creatinine+plasma Ba or plasma sC5b9; ( ) urine sC5b9+urine cystatin C/creatinine+plasma Ba or plasma sC5b9; (k) urine sC5b9+urine Ba/creatinine+plasma Ba or plasma sC5b9; (l) urine sC5b9+urine sC5b9/creatinine+plasma Ba or plasma sC5b9; (m) urine cystatin C/creatinine+urine Ba/creatinine+plasma Ba or plasma sC5b9; (n) urine cystatin C/creatinine+urine sC5b9/creatinine+plasma Ba or plasma sC5b9; (o) urine Ba/creatinine+urine sC5b9/creatinine+plasma Ba or plasma sC5b9.

A four biomarker signature from heterogeneous samples (e.g., including at least two from urine and two from plasma) may be employed in the diagnostic and therapeutic methods described herein. The exemplary four biomarker signatures from heterogeneous samples may be detected in fluid biological samples. Examples of heterogeneous four biomarker signatures include but are not limited to (a) urine cystatin C+urine Ba+plasma Ba+plasma sC5b9; (b) urine cystatin C+urine sC5b9+plasma Ba+plasma sC5b9; (c) urine cystatin C+urine cystatin C/creatinine+plasma Ba+plasma sC5b9; (d) urine cystatin C+urine Ba/creatinine+plasma Ba+plasma sC5b9; (e) urine cystatin C+urine sC5b9/creatinine+plasma Ba+plasma sC5b9; (f) urine Ba+urine sC5b9+plasma Ba+plasma sC5b9; (g) urine Ba+urine cystatin C/creatinine+plasma Ba+plasma sC5b9; (h) urine Ba+urine Ba/creatinine+plasma Ba+plasma sC5b9; (i) urine Ba+urine sC5b9/creatinine+plasma Ba+plasma sC5b9; j) urine sC5b9+urine cystatin C/creatinine+plasma Ba+plasma sC5b9; (k) urine sC5b9+urine Ba/creatinine+plasma Ba+plasma sC5b9; (l) urine sC5b9+urine sC5b9/creatinine+plasma Ba+plasma sC5b9; (m) urine cystatin C/creatinine+urine Ba/creatinine+plasma Ba+plasma sC5b9; (n) urine cystatin C/creatinine+urine sC5b9/creatinine+plasma Ba+plasma sC5b9; (o) urine Ba/creatinine+urine sC5b9/creatinine+plasma Ba+plasma sC5b9.

A four biomarker signature from heterogeneous samples (e.g., including at least three from urine and one from plasma) may be employed in the diagnostic and therapeutic methods described herein. The exemplary four biomarker signatures from heterogeneous samples may be detected in fluid biological samples. Examples of heterogeneous three biomarker signatures include but are not limited to (a) urine cystatin C+urine Ba+urine sC5b9+plasma Ba or plasma sC5b9; (b) cystatin C+Ba+cystatin C/creatinine+plasma Ba or plasma sC5b9; (c) cystatin C+Ba+Ba/creatinine+plasma Ba or plasma sC5b9; (d) cystatin C+Ba+sC5b9/creatinine+plasma Ba or plasma sC5b9; (e) cystatin C+sC5b9+cystatin C/creatinine+plasma Ba or plasma sC5b9; (f) cystatin C+sC5b9+Ba/creatinine+plasma Ba or plasma sC5b9; (g) cystatin C+sC5b9+sC5b9/creatinine+plasma Ba or plasma sC5b9; (h) cystatin C+cystatin C/creatinine+Ba/creatinine+plasma Ba or plasma sC5b9; (i) cystatin C+cystatin C/creatinine+sC5b9/creatinine+plasma Ba or plasma sC5b9; (j) cystatin C+Ba/creatinine+sC5b9/creatinine+plasma Ba or plasma sC5b9; (k) Ba+sC5b9+cystatin C/creatinine+plasma Ba or plasma sC5b9; (l) Ba+sC5b9+Ba/creatinine+plasma Ba or plasma sC5b9; (m) Ba+sC5b9+sC5b9/creatinine+plasma Ba or plasma sC5b9; (n) Ba+cystatin C/creatinine+Ba/creatinine+plasma Ba or plasma sC5b9; (o) Ba+cystatin C/creatinine+sC5b9/creatinine+plasma Ba or plasma sC5b9; (p) Ba+Ba/creatinine+sC5b9/creatinine+plasma Ba or plasma sC5b9; (q) sC5b9+cystatin C/creatinine+Ba/creatinine+plasma Ba or plasma sC5b9; (r) sC5b9+cystatin C/creatinine+sC5b9/creatinine+plasma Ba or plasma sC5b9; (s) sC5b9+Ba/creatinine+sC5b9/creatinine+plasma Ba or plasma sC5b9; (t) cystatin C/creatinine+Ba/creatinine+sC5b9/creatinine+plasma Ba or plasma sC5b9.

A four biomarker signature from a heterogeneous sample (e.g., two from serum, one from plasma, and one from urine) may be used in the diagnostic and therapeutic methods described herein. The exemplary four biomarker signatures from the heterogeneous samples described herein may be detected in fluid biological samples. An exemplary four biomarker signature may comprise serum sVCAM-1 (circulating vascular adhesion molecule-1), serum sTNF-R1 (soluble Tumor necrosis factor receptor 1), plasma thrombomodulin, and urine C5b-9.

A five biomarker signature from heterogeneous samples (e.g., including at least three from urine and two from plasma) may be employed in the diagnostic and therapeutic methods described herein. The exemplary five biomarker signatures from heterogeneous samples described herein may be detected in fluid biological samples. Examples of heterogeneous three biomarker signatures include but are not limited to the signatures selected from the group consisting of (a) urine cystatin C+urine Ba+urine sC5b9+plasma Ba or plasma sC5b9; (b) cystatin C+Ba+cystatin C/creatinine+plasma Ba or plasma sC5b9; (c) cystatin C+Ba+Ba/creatinine+plasma Ba or plasma sC5b9; (d) cystatin C+Ba+sC5b9/creatinine+plasma Ba or plasma sC5b9; (e) cystatin C+sC5b9+cystatin C/creatinine+plasma Ba or plasma sC5b9; (f) cystatin C+sC5b9+Ba/creatinine+plasma Ba or plasma sC5b9; (g) cystatin C+sC5b9+sC5b9/creatinine+plasma Ba or plasma sC5b9; (h) cystatin C+cystatin C/creatinine+Ba/creatinine+plasma Ba or plasma sC5b9; (i) cystatin C+cystatin C/creatinine+sC5b9/creatinine+plasma Ba or plasma sC5b9; (j) cystatin C+Ba/creatinine+sC5b9/creatinine+plasma Ba or plasma sC5b9; (k) Ba+sC5b9+cystatin C/creatinine+plasma Ba or plasma sC5b9; (l) Ba+sC5b9+Ba/creatinine+plasma Ba or plasma sC5b9; (m) Ba+sC5b9+sC5b9/creatinine+plasma Ba or plasma sC5b9; (n) Ba+cystatin C/creatinine+Ba/creatinine+plasma Ba or plasma sC5b9; (o) Ba+cystatin C/creatinine+sC5b9/creatinine+plasma Ba or plasma sC5b9; (p) Ba+Ba/creatinine+sC5b9/creatinine+plasma Ba or plasma sC5b9; (q) sC5b9+cystatin C/creatinine+Ba/creatinine+plasma Ba or plasma sC5b9; (r) sC5b9+cystatin C/creatinine+sC5b9/creatinine+plasma Ba or plasma sC5b9; (s) sC5b9+Ba/creatinine+sC5b9/creatinine+plasma Ba or plasma sC5b9; (t) cystatin C/creatinine+Ba/creatinine+sC5b9/creatinine+plasma Ba or plasma sC5b9, except that both plasma biomarkers, e.g., Ba and sC5b9, are included (the aforementioned heterogeneous four biomarker signatures include the plasma biomarkers in the alternate and not in a combination).

A five biomarker signature from heterogeneous samples (e.g., including at least four from urine and one from plasma) may be used in the methods described herein. The exemplary five biomarker signatures from heterogeneous samples described herein may be detected in fluid biological samples. Examples heterogeneous five biomarker signatures include but are not limited to a four biomarker signature from urine selected from the group consisting of (a) cystatin C+Ba+sC5b9+cystatin C/creatinine; (b) cystatin C+Ba+sC5b9+Ba/creatinine; (c) cystatin C+Ba+sC5b9+sC5b9/creatinine; (d) cystatin C+sC5b9+cystatin C/creatinine+(e) cystatin C+sC5b9+cystatin C/creatinine+sC5b9/creatinine; (f) cystatin C+cystatin C/creatinine+Ba/creatinine+sC5b9/creatinine; (g) Ba+sC5b9+cystatin C/creatinine+Ba/creatinine; (h) Ba+sC5b9+cystatin C/creatinine+sC5b9/creatinine; (i) Ba+cystatin C/creatinine+Ba/creatinine+sC5b9/creatinine; or (j) sC5b9+cystatin C/creatinine+Ba/creatinine+sC5b9/creatinine, and one biomarker from plasma selected from the group consisting of complement Ba and (b) sC5b9, or any combination thereof.

A six biomarker signature from heterogeneous samples (e.g., including at least four from urine and two from plasma) may be used in the methods described herein. The exemplary six biomarker signatures from heterogeneous samples described herein may be detected in fluid biological samples. Examples of heterogeneous six biomarker signatures may comprise, e.g., (1) a four biomarker signature from urine, selected from the group consisting of (a) cystatin C+Ba+sC5b9+cystatin C/creatinine; (b) cystatin C+Ba+sC5b9+Ba/creatinine; (c) cystatin C+Ba+sC5b9+sC5b9/creatinine; (d) cystatin C+sC5b9+cystatin C/creatinine+(e) cystatin C+sC5b9+cystatin C/creatinine+sC5b9/creatinine; (f) cystatin C+cystatin C/creatinine+Ba/creatinine+sC5b9/creatinine; (g) Ba+sC5b9+cystatin C/creatinine+Ba/creatinine; (h) Ba+sC5b9+cystatin C/creatinine+sC5b9/creatinine; (i) Ba+cystatin C/creatinine+Ba/creatinine+sC5b9/creatinine; or (j) sC5b9+cystatin C/creatinine+Ba/creatinine+sC5b9/creatinine and two biomarker signature from plasma, for example, Ba+sC5b9, above or (2) a five biomarker signature from urine selected from the group consisting of (a) cystatin C+Ba+sC5b9+cystatin C/creatinine+Ba/creatinine; (b) cystatin C+Ba+sC5b9+cystatin C/creatinine+sC5b9/creatinine; (c) cystatin C+sC5b9+cystatin C/creatinine+Ba/creatinine+sC5b9/creatinine; (d) Ba+sC5b9+cystatin C/creatinine+Ba/creatinine+sC5b9/creatinine, and a single biomarker from plasma selected from the group consisting of complement Ba and (b) sC5b9, or any combination thereof.

A seven biomarker signature from heterogeneous samples (e.g., including at least five from urine and two from plasma) may be used in the methods described herein. The exemplary seven biomarker signatures from heterogeneous samples described herein may be detected in fluid biological samples. Examples of heterogeneous seven biomarker signatures may comprise a five biomarker signature from urine selected from the group consisting of (a) cystatin C+Ba+sC5b9+cystatin C/creatinine+Ba/creatinine; (b) cystatin C+Ba+sC5b9+cystatin C/creatinine+sC5b9/creatinine; (c) cystatin C+sC5b9+cystatin C/creatinine+Ba/creatinine+sC5b9/creatinine; (d) Ba+sC5b9+cystatin C/creatinine+Ba/creatinine+sC5b9/creatinine and two biomarker signature from plasma, for example, Ba and sC5b9.

An eight biomarker signature from heterogeneous samples (e.g., including at least six from urine and two from plasma) may be used in the methods described herein. The exemplary eight biomarker signatures from heterogeneous samples described herein may be detected in fluid biological samples. Examples of heterogeneous eight biomarker signatures may comprise a six biomarker signature from urine comprising cystatin C+Ba+sC5b9+cystatin C/creatinine+Ba/creatinine+sC5b9/creatinine with two biomarker signature from plasma, for example, Ba and sC5b9.

The protein biomarkers described herein may be normalized, e.g., based on the level of creatinine in the subject's biological sample. For normalization, it is preferable to use the same source of the biological sample for determination of creatinine levels from which source the biomarker is derived. Preferably, the disclosure relates to normalization of urine biomarkers based on the level of urine creatinine levels (ng of biomarker per mg of creatinine). Especially, the levels of urine Ba, sC5b9 and cytostatin C are normalized on the basis of urine creatinine levels.

The biomarker levels may be normalized to levels of creatinine in the subject's sample, the creatinine levels are determined contemporaneously with the determination of the subject's biomarker levels or biomarker signatures (e.g., at the same time or separated by a very small time gap, e.g., a gap which is less than an hour, preferably less than 30 minutes, particularly less than 20 minutes, or especially less than 5 minutes). For example, the levels of plasma Ba and/or plasma sC5b9 may be normalized on the basis of plasma creatinine levels.

The diagnostic and/or therapeutic methods described herein may be comprise measuring at least one secondary marker together with the biomarkers and/or biomarker signatures described herein, wherein the biomarker levels are optionally normalized to creatinine levels in the subject's sample. Representative examples of secondary markers include, e.g., estimated glomerular filtration rates (eGFR) and/or urine protein creatinine ratio (UPCR). Preferably, these secondary markers are measured contemporaneously with the biomarkers or biomarker signatures, optionally together with levels of the normalizing analyte (creatinine levels).

Methods of Diagnosis and/or Detection

A method for detecting a biomarker in a biological sample may comprise obtaining a biological fluid from a subject; contacting the biological fluid with an agent that binds to a biomarker, e.g., a protein biomarker selected from a proteolytic fragment of complement component factor B (Ba), soluble C5b9 (sC5b-9), or a combination thereof; and detecting the binding of the agent to the biomarker, e.g., a protein biomarker selected from a proteolytic fragment of complement component factor B (Ba), soluble C5b9 (sC5b-9), or both. The method may further comprise determining the levels of Ba and/or sC5b-9. Any standard method may be employed in the detection of the biomarkers, including, antibody based detection comprising standard immunoassays such as enzyme-linked immunosorbent assay (ELISA), radio-immunoassay (RIA), or the like.

A method for monitoring or evaluating the status of CM-TMA-associated biomarker proteins in a subject may comprise obtaining a biological fluid from a subject; contacting the biological fluid with an agent that binds to a biomarker, e.g., a protein biomarker selected from a proteolytic fragment of complement component factor B (Ba), soluble C5b9 (sC5b-9), or a combination thereof; and detecting the binding of the agent to the biomarker, e.g., a protein biomarker selected from a proteolytic fragment of complement component factor B (Ba), soluble C5b9 (sC5b-9), or both. The method may further comprise determining the levels of Ba and/or sC5b-9.

A method for assessing one or both of the concentration and activity level of CM-TMA-associated biomarker protein in a subject may comprise obtaining a biological fluid, detecting the protein biomarker, e.g., a proteolytic fragment of complement component factor B (Ba), soluble C5b9 (sC5b-9), or both by an immunoassay. The method may further comprise determining the concentration of the biomarkers on the basis of standard determinations, e.g., calculating amount or relative amounts of biomarkers per unit volume of sample. For activity determinations, standard activity assays may be run. For instance, wherein the biomarker is complement Bb and the parameter of interest is the activity of Bb in the subject's sample, a C3 cleavage assay may be carried out. This assay recognizes that Bb is the catalytically active site of the C3bBb complex (C3 convertase) and is capable of cleaving new C3 to C3a and C3b. Similarly, wherein the biomarker is complement sC5b9 and the parameter of interest is the activity of sC5b9 in the subject's sample, a terminal complement complex assay such as CH50 may be used. The CH50 test is a lytic assay, which uses antibody-sensitized sheep erythrocytes (EA) as the activator of the classical complement pathway and various dilutions of the test serum to determine the amount required to give 50% lysis. The percent hemolysis is determined spectrophotometrically. The CH50 test is an indirect measure of TCC, since the TCC themselves are directly responsible for the hemolysis which is measured cleavage assay may be carried out. In some embodiments, a direct assay may be used.

A method for monitoring or determining whether a patient is at risk for developing thrombotic microangiopathy may comprise obtaining a biological fluid, detecting one or more of the foregoing biomarkers or biomarker signatures, e.g., proteolytic fragment of complement component factor B (Ba), soluble C5b9 (sC5b-9), or both, by an immunoassay. The method may further comprise determining the levels of Ba and/or sC5b-9.

A method for monitoring or evaluating the status of CM-TMA associated biomarker proteins in a subject or a method for assessing one or both of the concentration and activity level of one or more of the foregoing biomarkers or biomarker signatures, e.g., proteolytic fragment of complement component factor B (Ba), soluble C5b9 (sC5b-9), or both in a subject may comprise obtaining a biological fluid, detecting one or more of the foregoing biomarkers or biomarker signatures, e.g., a proteolytic fragment of complement component factor B (Ba), soluble C5b9 (sC5b-9), or both by an immunoassay. The method may further comprise determining the levels of Ba and/or sC5b-9.

A method for determining whether the subject has or is at risk for developing CM-TMA may comprise obtaining a biological fluid, detecting one or more of the foregoing biomarkers or biomarker signatures, e.g., a proteolytic fragment of complement component factor B (Ba), soluble C5b9 (sC5b-9), or both by an immunoassay. The method may further comprise determining the levels of Ba and/or sC5b-9.

A method for determining whether the subject has responded to treatment with a complement inhibitor may comprise obtaining a biological fluid, detecting one or more of the foregoing biomarkers or biomarker signatures, e.g., a proteolytic fragment of complement component factor B (Ba), soluble C5b9 (sC5b-9), or both by an immunoassay. The method may further comprise determining the levels of Ba and/or sC5b-9. In some embodiments, the disclosure relates to a method for treating complement-mediated thrombotic microangiopathy (CM-TMA) using a complement inhibitor in a manner sufficient to induce a physiological change in CM-TMA-associated biomarker proteins, the method comprising: (a) determining the level or activity of the CM-TMA-associated biomarker proteins in a biological fluid obtained from the subject, wherein the CM-TMA-associated biomarker proteins are selected from the group consisting of: cystatin C; cystatin C/creatinine ratio; complement factor Ba; complement factor Ba/creatinine ratio; complement sC5b-9; complement sC5b-9/creatinine ratio, or a combination thereof, preferably determining levels or activities of biomarkers in a signature comprising at least two biomarkers comprising Ba and sC5b9; and (b) administering to a subject having, suspected of having, or at risk for developing, CM-TMA, an inhibitor of complement in an amount and with a frequency sufficient to cause a reduction in the levels or activity of the biomarker or the biomarker signature, as compared to the level or activity thereof in a sample of biological fluid of the same type obtained from the subject prior to treatment with the inhibitor. In these embodiments, preferably the complement inhibitor comprises an anti-C5 antibody such as ravulizumab.

A method for determining whether a patient with complement-mediated thrombotic microangiopathy (CM-TMA) who is treated with a complement inhibitor under a predetermined dosing schedule is in need of a different dosing schedule may comprise (A) determining whether the CM-TMA patient is responsive to treatment with the complement inhibitor under the predetermined dosing schedule, wherein the determining comprises: determining one or both of the concentration and activity of CM-TMA-associated biomarker proteins in a biological fluid obtained from the subject, wherein the CM-TMA-associated biomarker proteins are selected from the group consisting of: cystatin C; cystatin C/creatinine ratio; complement factor Ba; complement factor Ba/creatinine ratio; complement sC5b-9; complement sC5b-9/creatinine ratio, or a combination thereof, preferably determining levels of biomarkers in a signature comprising at least two biomarkers comprising Ba and sC5b9, and wherein: (a) a reduced level or activity, as compared to the concentration in a sample of biological fluid of the same type obtained from the subject prior to treatment with the inhibitor, of the biomarker or the biomarker signature, indicates that the subject is responsive to treatment with the inhibitor; and (B) if the patient is not responsive to treatment with the complement inhibitor, administering the non-responsive patient a different complement inhibitor or the same complement inhibitor at a higher dose or more frequent dosing schedule as compared to the predetermined dosing schedule. In these embodiments, preferably the complement inhibitor comprises an anti-C5 antibody such as ravulizumab.

The subject may be a human having, suspected of having, or at risk for developing, aHUS. The subject may be a human having, suspected of having, or a risk for developing CM-TMA. The subject may be a human having, suspected of having, or a risk for developing lupus, optionally lupus nephritis.

The subject can be one who has been (or is being) treated with an inhibitor of complement, optionally an inhibitor of complement component C5, e.g., an anti-C5 antibody.

The treatment can have occurred less than one month (e.g., less than 31, 30, 29, 28, 27, 26, 25, 24, 23, 22, 21, 20, 19, 18, 17, 16, 15, 14, 13, 12, 11, 10, 9, 8, 7, 6, 5, 4, 3, 2, or 1 day) prior to obtaining the sample from the subject.

The methods described herein may further comprise a step of determining whether the subject has or is at risk of developing CM-TMA, particularly aHUS, Lupus, optionally lupus nephritis, or a combination thereof. Where the subject has been treated or is being treated with a complement inhibitor (e.g., an anti-C5 antibody) under a predetermined dosing schedule, the method may further comprise determining whether the patient is responsive to the complement inhibitor therapy.

A decreased concentration of a proteolytic fragment of complement component factor B (Ba) and soluble C5b9 (sC5b-9), as compared to the concentration of these biomarkers in a sample of biological fluid of the same type obtained from the subject prior to treatment with the inhibitor, indicates that the subject is responsive to treatment with the inhibitor.

A method for monitoring responsiveness of a subject to treatment with an inhibitor of complement component C5 may comprise detecting biomarkers in a biological fluid, wherein the biomarker is a proteolytic fragment of complement component factor B (Ba), soluble C5b9 (sC5b-9), or both. The biological fluid is obtained from a subject: (i) having, suspected of having, or at risk for developing, CM-TMA and (ii) who is being (or who has been, e.g., recently) treated with an inhibitor of complement component C5 under a predetermined dosing schedule. In accordance with such methods, (a) a reduced concentration, as compared to the concentration in a sample of biological fluid of the same type obtained from the subject prior to treatment with the inhibitor, of a proteolytic fragment of complement component factor B (Ba), soluble C5b9 (sC5b-9), or both; or (b) an increased concentration, as compared to the concentration in a sample of biological fluid of the same type obtained from the subject prior to treatment with the competitor of complement, indicates that the subject is responsive to treatment with the inhibitor.

A method for determining whether the subject has responded to treatment with the complement inhibitor may comprise detecting biomarkers in a biological fluid, wherein the biomarker is a proteolytic fragment of complement component factor B (Ba), soluble C5b9 (sC5b-9), or both. A reduced concentration, as compared to the concentration in a sample of biological fluid of the same type obtained from the subject prior to treatment with the inhibitor, of a proteolytic fragment of complement component factor B (Ba), soluble C5b9 (sC5b-9), or both.

A method for monitoring responsiveness of a subject to treatment with an inhibitor of complement may comprise detecting biomarkers in a biological fluid, wherein the biomarker is a proteolytic fragment of complement component factor B (Ba), soluble C5b9 (sC5b-9), or both. The subject has, is suspected of having, or is at risk for developing CM-TMA and the subject has been or is being treated with an inhibitor of complement. (A) a reduced concentration, as compared to the concentration in a sample of biological fluid of the same type obtained from the subject prior to treatment with the inhibitor, of detecting biomarkers in a biological fluid, wherein the biomarker is a proteolytic fragment of complement component factor B (Ba), soluble C5b9 (sC5b-9), or both; or (B) an increased concentration, as compared to the concentration in a sample of biological fluid of the same type obtained from the subject prior to treatment with the inhibitor of complement, indicates that the subject is responsive to treatment with the inhibitor.

A method for reducing the number, frequency, or occurrence, likelihood of occurrence, or risk of developing, lupus, optionally lupus nephritis, using a complement inhibitor in a manner sufficient to induce a physiological change in a biomarker protein associated with thrombosis or coagulation may comprise: (a) determining the concentration of a biomarker protein in a biological fluid obtained from the subject, wherein the biomarker proteins are a proteolytic fragment of complement component factor B (Ba), soluble C5b9 (sC5b-9), or both and relate to thrombosis and/or coagulation; and (b) administering to a subject having, suspected of having, or at risk for developing, lupus, optionally lupus nephritis an inhibitor of complement in an amount and with a frequency sufficient to cause a physiological change in a biomarker protein, wherein the physiological change is a reduction in the concentration of the biomarker protein relative to the concentration of the markers in an equivalent biological sample obtained from the subject prior to treatment with the complement inhibitor. The method may comprise both measuring the concentration of the biomarkers before and after treatment.

A method for determining whether an CM-TMA patient treated with a complement inhibitor under a predetermined dosing schedule is in need of: (i) treatment with a different complement inhibitor or (ii) treatment with the same complement inhibitor under a different dosing schedule may comprise: (A) determining whether the CM-TMA patient is responsive to treatment with the complement inhibitor under the predetermined dosing schedule, wherein the determining comprises: measuring in a biological fluid obtained from the subject one or both of the concentration and activity of biomarkers in a biological fluid, wherein the biomarker is a proteolytic fragment of complement component factor B (Ba), soluble C5b9 (sC5b-9), or both. in the biological fluid, and wherein: (a) a reduced concentration, as compared to the concentration in a sample of biological fluid of the same type obtained from the subject prior to treatment with the inhibitor, of a proteolytic fragment of complement component factor B (Ba), soluble C5b9 (sC5b-9), or both; or (b) an increased concentration, as compared to the concentration in a sample of biological fluid of the same type obtained from the subject prior to treatment with the inhibitor of complement indicates that the subject is responsive to treatment with the inhibitor; and (B) if the patient is not responsive to treatment with the complement inhibitor, administering the patient a different complement inhibitor or the same complement inhibitor at a higher dose or more frequent dosing schedule as compared to the predetermined dosing schedule.

A method for diagnosing a subject as having, or being at risk for developing, atypical hemolytic uremic syndrome (aHUS) may comprise measuring in a biological fluid the concentration of at least two aHUS-associated biomarker proteins a proteolytic fragment of complement component factor B (Ba), soluble C5b9 (sC5b-9), or both. The biological fluid is one obtained from a subject suspected of having or at risk for developing aHUS. In accordance with the methods, an elevated concentration, as compared to the concentration in a normal control biological fluid of the same type, of a proteolytic fragment of complement component factor B (Ba), soluble C5b9 (sC5b-9), or both, indicates that the subject has, or is at risk for developing, aHUS. A control may be obtained from a healthy subject, e.g., a subject who does not have CM-TMA, preferably a subject whose urine sample does not contain detectable levels of sC5b9, as measured via a standard immunoassay

A method for determining whether an CM-TMA patient has responded to therapy with a complement inhibitor may comprise measuring the concentration of Ba and/or sC5b-9, in a biological sample obtained from a patient having, suspected of having, or at risk for developing, CM-TMA and treated with a complement inhibitor (e.g., an anti-C5 antibody); and determining that the patient has responded to the therapy if the concentration of the one or more biomarkers in the biological sample is reduced, as compared to the concentration of the one or more biomarkers in a biological sample of the same type obtained from the patient prior to treatment with the complement inhibitor or determining that the patient has not responded to the therapy if the concentration of the one or more biomarkers in the biological sample is not reduced, as compared to the concentration of the one or more biomarkers in a biological sample of the same type obtained from the patient prior to treatment with the complement inhibitor. Thus, the method can be used to assess or monitor terminal complement blockade in an CM-TMA patient treated with a complement inhibitor. In embodiments in which the patient is non-responsive, or less responsive to therapy, the method can also include changing the dose amount or dose frequency of the complement inhibitor or electing a different complement inhibitor (e.g., an inhibitor of C3 activation) for use in treating the patient.

### Detection Methods

The biomarkers, a proteolytic fragment of complement component factor B (Ba), soluble C5b9 (sC5b-9), or both, can be measured using an immunoassay, e.g., enzyme linked immunosorbent assay (ELISA), a radioimmunoassay (RIA), fluoroimmunoassay (FIA), chemiluminescent immunoassay (CLIA), counting immunoassay (CIA), colorimetric immunoassay, Western blotting, dot blotting, cytometric bead array (CBA), or a combination thereof. Methodology for immunoassays are known in the art. Laboratory Methods in Immunology Shawkatová (2019).

Antibodies that bind to C5b as well as methods for making such antibodies are known in the art. Commercially available anti-C5b antibodies are available from a number of vendors including, e.g., Hycult Biotechnology (catalogue number: HM2080; clone 568) and ABCAM®. (ab46151 or ab46168).

The antibody may be an anti-factor B antibody (such as the monoclonal antibody 1379 produced by ATCC Deposit No. PTA-6230). Anti-factor B antibodies are also described in, e.g., Ueda et al. (1987) J Immunol 138(4): 1143-9; Tanhehco et al. (1999) Transplant Proc 31(5):2168-71; U.S. Pat. Nos. 7,999,082 and 7,964,705; and WO 09/029669.

In the Examples section and elsewhere, representative types of antibodies which are useful in carrying out various embodiments of the disclosure are provided, e.g., with information on the particular vendor and/or catalog number. It should be understood that the disclosure is not limited to the exemplary embodiments which utilize antibody detection regents from a particular vendor/manufacturer. Antibodies against the biomarkers/analytes of the disclosure can be obtained from any manufacturer, including, Biolegend (San Diego, CA), Southern Biotech (Birmingham, AL), United States Biological (USB; Salem, MA), Lifespan Biosciences (LSBIO; Seattle, WA), ABCAM® (Cambridge, United Kingdom), Cell Signaling Technology (Danvers, MA), and Sigma-Aldrich (St. Louis, MO). For example, rabbit anti-PODXL antibody can be purchased form USB (Catalog #212672), LSBIO (Catalog #LS-C141161), ABCAM® (Catalog #ab205350) and Sigma-Aldrich (Catalog #HPA002110); anti-CD9 antibody, clone MM2/57 can be purchased from Southern Biotech (Catalog #9310), EMD Millipore (Catalog #CBL162), VWR (Catalog #89366), and BIO RAD (Catalog #MCA469G). Antibodies may also be generated using conventional techniques, e.g., immunization of a mammal such as a mouse or rabbit and/or hybridoma technology.

The biomarkers may be detected using an array. For example, the array may be a protein chip where each address of the array is a well of an assay plate. Each address of the array may be a particle (e.g., a bead) having immobilized thereupon a binding agent.

Measuring protein expression levels in a biological sample may be performed by any suitable method. See, e.g., Greenfield (Ed.) (2014) “Antibodies: A Laboratory Manual”, Cold Spring Harbor Laboratory: Cold Spring Harbor, N.Y. In general, protein levels are determined by contacting a biological sample obtained from a subject with binding agents for the biomarker proteins; detecting, in the sample (e.g., the biological fluid), the levels of one or more of the biomarker proteins that bind to the binding agents; and comparing the levels of one or more of the biomarker proteins in the sample with the levels of the corresponding protein biomarkers in a control sample (e.g., a normal sample). In certain embodiments, a suitable binding agent is a ribosome, with or without a peptide component, an RNA molecule, or a polypeptide (e.g., a polypeptide that comprises a polypeptide sequence of a protein marker, a peptide variant thereof, or a non-peptide mimetic of such a sequence).

Suitable binding agents also include an antibody specific for a biomarker protein described herein. Suitable antibodies for use in the methods of the present invention include monoclonal and polyclonal antibodies and antigen-binding fragments (e.g., Fab fragments or scFvs) of antibodies. Antibodies, including monoclonal and polyclonal antibodies, fragments and chimeras, may be prepared using methods known in the art. Antibodies to be used in the methods of the invention can be purified by methods well known in the art. Greenfield (Ed.) (2014) “Antibodies: A Laboratory Manual”, Cold Spring Harbor Laboratory: Cold Spring Harbor, N.Y. Antibodies may also be obtained from commercial sources.

The binding agent is directly or indirectly labeled with a detectable moiety. The role of a detectable agent is to facilitate the detection step of the diagnostic method by allowing visualization of the complex formed by binding of the binding agent to the protein marker (or fragment thereof). The detectable agent can be selected such that it generates a signal that can be measured and whose intensity is related (preferably proportional) to the amount of protein marker present in the sample being analyzed. Methods for labeling biological molecules such as polypeptides and antibodies are well-known in the art. Any of a wide variety of detectable agents can be used in the practice of the present invention. Suitable detectable agents include, but are not limited to: various ligands, radionuclides, fluorescent dyes, chemiluminescent agents, microparticles (e.g., quantum dots, nanocrystals, phosphors), enzymes (e.g., those used in an ELISA, e.g., horseradish peroxidase, beta-galactosidase, luciferase, alkaline phosphatase), colorimetric labels, magnetic labels, and biotin, digoxigenin or other haptens and proteins for which antisera or monoclonal antibodies are available.

The binding agents (e.g., antibodies) may be immobilized on a carrier or support (e.g., a bead, a magnetic particle, a latex particle, a microtiter plate well, a cuvette, or other reaction vessel). Examples of suitable carrier or support materials include agarose, cellulose, nitrocellulose, dextran, Sephadex®, Sepharose®, liposomes, carboxymethyl cellulose, polyacrylamides, polystyrene, gabbros, filter paper, magnetite, ion-exchange resin, plastic film, plastic tube, glass, polyamine-methyl vinyl-ether-maleic acid copolymer, amino acid copolymer, ethylene-maleic acid copolymer, nylon, silk, or combinations thereof. Binding agents may be indirectly immobilized using second binding agents specific for the first binding agents (e.g., mouse antibodies specific for the protein markers may be immobilized using sheep anti-mouse IgG Fc fragment specific antibody coated on the carrier or support).

Protein expression levels in a biological sample may be determined using immunoassays. Examples of such assays are time resolved fluorescence immunoassays (TR-FIA), radioimmunoassays, enzyme immunoassays (e.g., ELISA), immunofluorescence immunoprecipitation, latex agglutination, hemagglutination, Western blot, and histochemical tests, which are conventional methods well-known in the art. Methods of detection and quantification of the signal generated by the complex formed by binding of the binding agent with the protein marker will depend on the nature of the assay and of the detectable moiety (e.g., fluorescent moiety).

In an example, the presence or amount of protein expression of a gene (e.g., Ba and/or sC5b-9) can be determined using a Western blotting technique. For example, a lysate can be prepared from a biological sample, or the biological sample (e.g., biological fluid) itself, can be contacted with Laemmli buffer and subjected to sodium-dodecyl sulfate polyacrylamide gel electrophoresis (SDS-PAGE). SDS-PAGE-resolved proteins, separated by size, can then be transferred to a filter membrane (e.g., nitrocellulose) and subjected to immunoblotting techniques using a detectably-labeled antibody specific to the protein of interest. The presence or amount of bound detectably-labeled antibody indicates the presence or amount of protein in the biological sample.

In an example, an immunoassay can be used for detecting and/or measuring the protein expression of a biomarker protein (e.g., Ba and/or sC5b-9). As above, for the purposes of detection, an immunoassay can be performed with an antibody that bears a detection moiety (e.g., a fluorescent agent or enzyme). Proteins from a biological sample can be conjugated directly to a solid-phase matrix (e.g., a multi-well assay plate, nitrocellulose, agarose, Sepharose®, encoded particles, or magnetic beads) or it can be conjugated to a first member of a specific binding pair (e.g., biotin or streptavidin) that attaches to a solid-phase matrix upon binding to a second member of the specific binding pair (e.g., streptavidin or biotin). Such attachment to a solid-phase matrix allows the proteins to be purified away from other interfering or irrelevant components of the biological sample prior to contact with the detection antibody and also allows for subsequent washing of unbound antibody. Here, as above, the presence or amount of bound detectably-labeled antibody indicates the presence or amount of protein in the biological sample.

Alternatively, the protein expression levels may be determined using mass spectrometry based methods or image-based methods known in the art for the detection of proteins. Other suitable methods include 2D-gel electrophoresis, proteomics-based methods such as the identification of individual proteins recovered from the gel (e.g., by mass spectrometry or N-terminal sequencing) and/or bioinformatics.

Methods for detecting or measuring protein expression can, optionally, be performed in formats that allow for rapid preparation, processing, and analysis of multiple samples. This can be, for example, in multi-well assay plates (e.g., 96 wells or 386 wells) or arrays (e.g., protein chips). Stock solutions for various reagents can be provided manually or robotically, and subsequent sample preparation, pipetting, diluting, mixing, distribution, washing, incubating (e.g., hybridization), sample readout, data collection (optical data) and/or analysis (computer aided image analysis) can be done robotically using commercially available analysis software, robotics, and detection instrumentation capable of detecting the signal generated from the assay. Examples of such detectors include, but are not limited to, spectrophotometers, luminometers, fluorimeters, and devices that measure radioisotope decay.

### Diagnostic Antibodies

The antibody or antigen-binding fragment thereof may be selected from the group consisting of a humanized antibody, a recombinant antibody, a diabody, a chimerized or chimeric antibody, a monoclonal antibody, a deimmunized antibody, a fully human antibody, a single chain antibody, an Fv fragment, an Fd fragment, an Fab fragment, an Fab′ fragment, an F(ab′)₂ fragment, or a combination thereof.

### Monoclonal Antibodies

The monoclonal antibodies disclosed herein can be of any isotype. The monoclonal antibody can be, for example, an IgM or an IgG antibody, such as IgG1 or an IgG2. The class of an antibody that immunospecifically binds Ba or sC5b-9 can be switched with another (for example, IgG can be switched to IgM), according to well-known procedures. Class switching can also be used to convert one IgG subclass to another, such as from IgG1 to IgG2.

The antibodies of the present invention may be monovalent, bivalent, trivalent or multivalent. For example, monovalent scFvs can be multimerized either chemically or by association with another protein or substance. An scFv that is fused to a hexahistidine tag or a Flag tag can be multimerized using Ni-NTA agarose (Qiagen) or using anti-Flag antibodies (Stratagene, Inc.).

The antibodies of the present invention may be monospecific, bispecific, trispecific or of greater multispecificity. Multispecific antibodies may be specific for different epitopes of Ba and/or sC5b-9, or fragment thereof, and a heterologous epitope, such as a heterologous polypeptide or solid support material. See, e.g., WO 93/17715; WO 92/08802; WO 91/00360; WO 92/05793; Tutt et al, J. Immunol. 147:60-69 (1991); U.S. Pat. Nos. 4,474,893; 4,714,681; 4,925,648; 5,573,920; 5,601,819; Kostelny et al. J. Immunol. 148:1547-1553 (1992).

### Methods for Producing Antibodies

Antibodies that may be used in the methods described herein (including scFvs and other molecules comprising, or alternatively consisting of antibody fragments or variants of the invention) can be produced by any method known in the art for the synthesis of antibodies, in particular, by chemical synthesis or preferably, by recombinant expression techniques. Greenfield (Ed.) (2014) “Antibodies: A Laboratory Manual”, Cold Spring Harbor Laboratory: Cold Spring Harbor, N.Y.

Single chain Fvs (scFvs) that immunospecifically bind Ba and/or sC5b-9, or fragment thereof may be generated using phage display methods known in the art. In phage display methods, functional antibody domains are displayed on the surface of phage particles which carry the polynucleotide sequences encoding them. In particular, DNA sequences encoding VH and VL domains are amplified from animal cDNA libraries (e.g., human or murine cDNA libraries of lymphoid tissues) or synthetic cDNA libraries. The DNA encoding the VH and VL domains are joined together by an scFv linker by PCR and cloned into a phagemid vector (e.g., p CANT AB 6 or pComb 3 HSS). The vector is electroporated in E. coli and the E. coli is infected with helper phage. Phage used in these methods are typically filamentous phage including fd and M13 and the VH and VL domains are usually recombinantly fused to either the phage gene III or gene VIII. Phage expressing an antigen binding domain that binds to an antigen of interest (e.g., Ba and/or sC5b-9, or fragment thereof) can be selected or identified with antigen, e.g., using labeled antigen or antigen bound or captured to a solid surface or bead. Examples of phage display methods that can be used to make the antibodies of the present invention include, but are not limited to, those disclosed in Brinkman et al., J. Immunol. Methods 182:41-50 (1995); Ames et al., J. Immunol. Methods 184:177-186 (1995); Kettleborough et al. Eur. J. Immunol. 24:952-958 (1994); Persic et al., Gene 187 9-18 (1997); Burton et al. Advances in Immunology 57:191-280(1994); WO 91/10737; WO 92/01047; WO 92/18619; WO 93/11236; WO 95/15982; WO 95/20401; WO97/13844; and U.S. Pat. Nos. 5,698,426; 5,223,409; 5,403,484; 5,580,717; 5,427,908; 5,750,753; 5,821,047; 5,571,698; 5,427,908; 5,516,637; 5,780,225; 5,658,727; 5,733,743 and 5,969,108.

As described in the above references, after phage selection, the antibody coding regions from the phage can be isolated and used to generate whole antibodies, including human or humanized antibodies, or any other desired antigen binding fragment, and expressed in any desired host, including mammalian cells, insect cells, plant cells, yeast, and bacteria, e.g., as described below. Techniques to recombinantly produce Fab, Fab′ and F(ab′)2 fragments can also be employed using methods known in the art such as those disclosed in WO 92/22324; Mullinax et al., BioTechniques 12(6):864-869 (1992); Sawai et al., AJRI 34:26-34 (1995); and Better et al., Science 240:1041-1043 (1988).

To generate whole antibodies, PCR primers including VH or VL nucleotide sequences, a restriction site, and a flanking sequence to protect the restriction site can be used to amplify the VH or VL sequences in scFv clones. Utilizing cloning techniques known to those of skill in the art, the PCR amplified VH domains can be cloned into vectors expressing a VH constant region, e.g., the human gamma 4 constant region, and the PCR amplified VL domains can be cloned into vectors expressing a VL constant region, e.g., human kappa or lambda constant regions. Preferably, the vectors for expressing the VH or VL domains comprise a promoter suitable to direct expression of the heavy and light chains in the chosen expression system, a secretion signal, a cloning site for the immunoglobulin variable domain, immunoglobulin constant domains, and a selection marker such as neomycin. The VH and VL domains may also be cloned into one vector expressing the necessary constant regions. The heavy chain conversion vectors and light chain conversion vectors are then co-transfected into cell lines to generate stable or transient cell lines that express full-length antibodies, e.g., IgG, using techniques known to those of skill in the art.

Once an antibody that may be used in the methods described herein (including molecules comprising, or alternatively consisting of, antibody fragments or variants thereof) has been chemically synthesized or recombinantly expressed, it may be purified by any method known in the art for purification of an immunoglobulin molecule, or more generally, a protein molecule, such as, for example, by chromatography (e.g., ion exchange, affinity, particularly by affinity for the specific antigen after Protein A, and sizing column chromatography), centrifugation, differential solubility, or by any other standard technique for the purification of proteins. Further, the antibodies that may be used in the methods described herein may be fused to heterologous polypeptide sequences described herein or otherwise known in the art, to facilitate purification.

Methods for recombinantly producing antibodies that may be used in the methods described herein are well known to those of ordinary skill in the art. The antibodies may also be produced by constructing, using conventional techniques well known to those of ordinary skill in the art, an expression vector comprising an operon and a DNA sequence encoding the antibodies. Furthermore, the invention relates to vectors, especially plasmids, cosmids, viruses, bacteriophages and other vectors common in genetic engineering, which may comprise the above-mentioned nucleic acid molecules. The nucleic acid molecules contained in the vectors may be linked to regulatory elements that ensure the transcription in prokaryotic and eukaryotic cells.

Vectors comprise elements that facilitate manipulation for the expression of a foreign protein within the target host cell. Conveniently, manipulation of sequences and production of DNA for transformation is first performed in a bacterial host (e.g., E. coli) and usually vectors will include sequences to facilitate such manipulations, including a bacterial origin of replication and appropriate bacterial selection marker. Selection markers encode proteins necessary for the survival or growth of transformed host cells grown in a selective culture medium. Host cells not transformed with the vector containing the selection gene will not survive in the culture medium. Typical selection genes encode proteins that confer resistance to antibiotics or other toxins, complement auxotrophic deficiencies, or supply critical nutrients not available from complex media. Exemplary vectors and methods for transformation of yeast are described in the art. See, e.g., Burke, et al. (2000) Methods in Yeast Genetics Cold Spring Harbor Laboratory Press.

The polynucleotide coding the antibodies may be operably linked to transcriptional and translational regulatory sequences that provide for expression of the polypeptide in yeast cells. These vector components may include, but are not limited to, one or more of the following: an enhancer element, a promoter, and a transcription termination sequence. Sequences for the secretion of the polypeptide may also be included (e.g., a signal sequence).

Nucleic acids are “operably linked” when placed into a functional relationship with another nucleic acid sequence. For example, DNA for a signal sequence is operably linked to DNA for a polypeptide if it is expressed as a preprotein that participates in the secretion of the polypeptide; a promoter or enhancer is operably linked to a coding sequence if it affects the transcription of the sequence. Generally, “operably linked” refers broadly to contiguous linked DNA sequences, and, in the case of a secretory leader, contiguous and in reading frame. However, enhancers do not have to be contiguous.

Promoters are untranslated sequences located upstream (5′) to the start codon of a structural gene (generally within about 100 to 1000 bp) that control the transcription and translation of particular nucleic acid sequences to which they are operably linked. Such promoters fall into several classes: inducible, constitutive, and repressible promoters (e.g., that increase levels of transcription in response to absence of a repressor). Inducible promoters may initiate increased levels of transcription from DNA under their control in response to some change in culture conditions (e.g., the presence or absence of a nutrient or a change in temperature.)

The expression vectors are transfected into a host cell by convention techniques well known to those of ordinary skill in the art to produce a transfected host cell, said transfected host cell cultured by conventional techniques well known to those of ordinary skill in the art to produce said antibodies.

The host cells used to express the anti-Ba and/or anti-sC5b-9 antibodies may be either a bacterial cell such as E. coli, yeast (e.g., S. cerevisiae), or a eukaryotic cell (e.g., a mammalian cell line). A mammalian cell of a well-defined type for this purpose, such as a myeloma cell, 3T3, HeLa, C6A2780, Vero, MOCK II, a Chinese hamster ovary (CHO), Sf9, Sf21, COS, NSO, or HEK293 cell line may be used.

The general methods by which the vectors may be constructed, transfection methods required to produce the host cell and culturing methods required to produce the antibodies, and fragments thereof, from said host cells all include conventional techniques. Although preferably the cell line used to produce the antibodies is a mammalian cell line, any other suitable cell line, such as a bacterial cell line such as an E. coli-derived bacterial strain, or a yeast cell line, may be used.

Similarly, once produced the antibodies may be purified according to standard procedures in the art, such as for example cross-flow filtration, ammonium sulphate precipitation, and affinity column chromatography.

Antibodies binding to biomarkers or biomarker signatures associated with CM-TMA may be screened using any known methods, e.g., binding assays. In a representative method, target biomarkers or antigenic epitope thereof are expressed in standard cells and antibodies are panned using selection techniques known in the art. Antibodies may be ranked, e.g., based on binding affinities, for example, a dissociation constant (K_{d}) of at least 10⁻⁶ M; preferably 10⁻⁸ M; and especially 10⁻¹⁰ M. Here, K_{d} values may be determined using standard binding assays.

### Biological Fluids

Suitable biological samples for use in the methods described herein include, e.g., any biological fluid. A biological sample can be, for example, a specimen obtained from a subject (e.g., a mammal such as a human) or can be derived from such a subject. A biological sample can also be a biological fluid such as urine, whole blood or a fraction thereof (e.g., plasma or serum), saliva, semen, sputum, cerebrospinal fluid, tears, or mucus. A biological sample can be further fractionated, if desired, to a fraction containing particular analytes (e.g., proteins) of interest. For example, a whole blood sample can be fractionated into serum or into fractions containing particular types of proteins. If desired, a biological sample can be a combination of different biological samples from a subject such as a combination of two different fluids.

The biological fluid obtained from a patient may be blood, optionally a blood fraction, including but not limited to, serum or plasma. The biological fluid may be urine. The biological fluid may be blood, serum, plasma, urine, or a combination thereof. The measurements may be performed on one biological fluid. In the methods described herein, the measurements may be performed on at least two different biological fluids obtained from the subject, e.g., a combination of blood, plasma, serum, and urine. For example, the concentration a first biomarker protein is measured in one type of biological fluid and the second biomarker protein is measured in a second type of biological fluid.

Biological samples suitable for the invention may be fresh or frozen samples collected from a subject, or archival samples with known diagnosis, treatment and/or outcome history. The biological samples can be obtained from a subject, e.g., a subject having, suspected of having, or at risk of developing, a complement-associated disorder (e.g., CM-TMA). Any suitable methods for obtaining the biological samples can be employed, although exemplary methods include, e.g., phlebotomy, swab (e.g., buccal swab), lavage, or fine needle aspirate biopsy procedure.

A protein extract may be prepared from a biological sample. A protein extract contains the total protein content. Methods of protein extraction are well known in the art. See, e.g., Roe (2001) “Protein Purification Techniques: A Practical Approach”, 2^{nd} Edition, Oxford University Press. Numerous different and versatile kits can be used to extract proteins from bodily fluids and tissues, and are commercially available from, for example, BioRad Laboratories (Hercules, Calif.), BD Biosciences Clontech (Mountain View, Calif.), Chemicon International, Inc. (Temecula, Calif.), Calbiochem (San Diego, Calif.), Pierce Biotechnology (Rockford, Ill.), and Invitrogen Corp. (Carlsbad, Calif.).

Methods for obtaining and/or storing samples that preserve the activity or integrity of biomarkers in the biological sample are well known to those skilled in the art. For example, a biological sample can be further contacted with one or more additional agents such as appropriate buffers and/or inhibitors, including protease inhibitors, the agents meant to preserve or minimize changes (e.g., changes in osmolarity or pH) in protein structure. Such inhibitors include, but are not limited to, chelators such as ethylenediamine tetraacetic acid (EDTA), ethylene glycol tetraacetic acid (EGTA), protease inhibitors such as phenylmethylsulfonyl fluoride (PMSF), aprotinin, and leupeptin. Appropriate buffers and conditions for storing or otherwise manipulating samples are described in, e.g., Pollard and Walker (1997), “Basic Cell Culture Protocols,” volume 75 of Methods in molecular biology, Humana Press; Masters (2000) “Animal cell culture: a practical approach,” volume 232 of Practical approach series, Oxford University Press; and Jones (1996) “Human cell culture protocols,” volume 2 of Methods in molecular medicine, Humana Press.

A sample can be processed to eliminate or minimize the presence of interfering substances. For example, a biological sample can be fractionated or purified to remove one or more materials (e.g., cells) that are not of interest. Methods of fractionating or purifying a biological sample include, but are not limited to, flow cytometry, fluorescence activated cell sorting, and sedimentation.

The methods described herein may comprise recording the measured value(s) of the concentration of the biomarker protein(s). The recordation can be written or on a computer readable medium. The method may further comprise communicating the measured value(s) of the concentration of the biomarker protein to the subject and/or to a medical practitioner in whose care the subject is placed.

### Therapeutic Methods for Treating CM-TMA

“Complete TMA response,” refers broadly to a composite outcome measure that required normalization of hematological parameters (e.g., platelet count and lactate dehydrogenase [LDH]) and improvement in kidney function (≥25% reduction in serum creatinine from baseline); for participants on dialysis, baseline was established at least 6 days after the end of dialysis. Participants had to meet these criteria for 2 separate assessments obtained at least 4 weeks (28 days) apart, and any measurement in between during a 26-week Initial Evaluation Period. The inventors surprisingly discovered that, lower baseline levels of (1) serum sVCAM-1, (2) serum sTNF-R1, (3) plasma thrombomodulin and (4) urine sC5b-9, were associated with higher likelihood of achieving complete TMA response.

Additionally, a complete TMA response may be assessed by the combined measure of normalization of platelet count and LDH, along with improvement in serum creatinine in the patient receiving the treatment.

The complement inhibitor may be administered to the subject under a predetermined dosing schedule based, in part, on the body weight of the subject. Exemplary anti-C5 antibody dosing schedules (e.g., chronic dosing schedules) are described in WO 2010/054403 and U.S. Pat. No. 9,658,236.

The complement inhibitor may be antibody or an antigen binding fragment thereof, a small molecule, a polypeptide, a polypeptide analog, a peptidomimetic, or an aptamer. The complement inhibitor may inhibit one or more of complement components C1, C2, C3, C4, C5, C6, C7, C8, C9, Factor D, Factor B, properdin, MBL, MASP-1, MASP-2, a biologically active fragment, or a combination thereof. The complement inhibitor may inhibit one or both of the generation of the anaphylatoxic activity associated with C5a and/or the assembly of the membrane attack complex associated with C5b.

Naturally occurring or soluble forms of complement inhibitory compounds including but not limited to CR1, LEX-CRI, MCP, DAF, CD59, Factor H, cobra venom factor, FUT-175, complestatin, K76 COOH, and combinations thereof, may be used.

The complement inhibitor may be a complement receptor 2 (CR2)-factor H (FH) molecule comprising: a) a CR2 portion comprising CR2 (e.g., human CR2) or a fragment thereof, and b) a FH portion comprising a FH or a fragment thereof, wherein the CR2-FH molecule or fragment thereof is capable of binding to a CR2 ligand, and wherein the CR2-FH molecule is capable of inhibiting complement activation of the alternative pathway. Exemplary CR2-FH fusion proteins are described in WO 2007/149567 and WO 2011/143637. The complement inhibitor may comprise a targeting domain such as CR2 or an anti-C3d antibody as described in WO 2011/163412. Fusions of targeting domains with other complement inhibitors such as CD59, CD55, and factor H-like molecules can be used in the methods described herein as a complement inhibitor. WO 2011/163412.

The inhibitor of complement may be selected from the group consisting of recombinant anti-C5 mini-antibody MB12/22, anti-C5 mini-antibody targeted to the endothelium MB12/22-RGD, C5 specific aptamer ARC187, C5 specific aptamer ARC1905 (Avacincaptad pegol), Staphylococcal superantigen-like protein 7 (SSL7), Ornithodoros moubata C inhibitor (OmCl), or a combination thereof.

The inhibitor of complement may be an antagonist antibody or antigen-binding fragment thereof. The antibody or antigen-binding fragment thereof may be selected from the group consisting of a humanized antibody, a recombinant antibody, a diabody, a chimerized or chimeric antibody, a monoclonal antibody, a deimmunized antibody, a fully human antibody, a single chain antibody, an Fv fragment, an Fd fragment, an Fab fragment, an Fab′ fragment, an F(ab′)₂ fragment, or a combination thereof.

The antagonist antibody may be an anti-C5 antibody including but not limited to SOLIRIS® (eculizumab) and ULTOMIRIS® (ravulizumab). The antagonist antibody may be pexelizumab, a C5-binding fragment of anti-C5 antibody.

The methods described herein may comprise a step of administering to the subject the complement inhibitor at a higher dose or with an increased frequency of dosing, relative to the predetermined dosing schedule, if the subject is not responsive to treatment with the inhibitor under the predetermined dosing schedule.

A method for treating complement-mediated thrombotic microangiopathy (CM-TMA) may comprise administering to a subject having, suspected of having, or at risk for developing, CM-TMA an inhibitor of complement (e.g., an inhibitor of complement component C5) in an amount and with a frequency sufficient to effect a physiological change in a proteolytic fragment of complement component factor B (Ba), soluble C5b9 (sC5b-9), or both, wherein the physiological change is selected from the group consisting of: (a) a reduced concentration, as compared to the concentration in a sample of biological fluid of the same type obtained from the subject prior to treatment with the inhibitor, of a proteolytic fragment of complement component factor B (Ba), soluble C5b9 (sC5b-9), or both; or (b) an increased concentration, as compared to the concentration in a sample of biological fluid of the same type obtained from the subject prior to treatment with the inhibitor of complement. In some embodiments, the at least one CM-TMA-associated biomarker can be a proteolytic fragment of complement component factor B (Ba), soluble C5b9 (sC5b-9), or both.

A method for treating complement-mediated thrombotic microangiopathy (CM-TMA) using a complement inhibitor in a manner sufficient to induce a physiological change in CM-TMA-associated biomarker proteins comprising: (a) determining the concentration of a CM-TMA-associated biomarker proteins in a biological fluid obtained from the subject, wherein the CM-TMA-associated biomarker proteins are a proteolytic fragment of complement component factor B (Ba), soluble C5b9 (sC5b-9), or both; and (b) administering to a subject having, suspected of having, or at risk for developing, CM-TMA an inhibitor of complement in an amount and with a frequency sufficient to cause a physiological change in at least each of two (2) CM-TMA-associated biomarker proteins, wherein the physiological change is selected from the group consisting of: (a) a reduced concentration, as compared to the concentration in a sample of biological fluid of the same type obtained from the subject prior to treatment with the inhibitor, of a proteolytic fragment of complement component factor B (Ba), soluble C5b9 (sC5b-9), or both; and (b) an increased concentration in a biological fluid of obtained from the subject, as compared to the concentration in a sample of biological fluid of the same type obtained from the subject prior to treatment with the inhibitor of complement. The method can also comprise determining whether the physiological changes occurred.

A method for detecting a set of biomarkers in a subject may comprise detecting the level of at least 1, at least 2, at least 3, at least 4, at least 5, at least 6 biomarker(s) selected from the group consisting of cystatin C; cystatin C/creatinine ratio; complement factor Ba; complement factor Ba/creatinine ratio; complement sC5b-9; and/or complement sC5b-9/creatinine ratio; preferably detecting levels of biomarkers in a subset comprising at least two biomarkers comprising Ba and sC5b9, in a fluid biological sample obtained from the subject.

The methods described herein may further comprise measuring the concentrations of CM-TMA-associated biomarker proteins in a biological fluid, wherein the CM-TMA-associated biomarker proteins are a proteolytic fragment of complement component factor B (Ba), soluble C5b9 (sC5b-9), or both. The biological fluid may be obtained from the subject.

The method described herein may comprise determining whether the proteolytic fragment of complement component factor B (Ba), soluble C5b9 (sC5b-9), or both physiological changes have occurred. After treatment, the concentrations of both Ba and sC5b9 may be reduced. For example, after treatment, the concentration (e.g., the urine, blood, plasma, serum concentration) of each of Ba and sC5b9 may be reduced.

The Ba concentration may be reduced by at least 10% by week 6 post-initiation of treatment. The Ba concentration may be reduced by at least 30% by week 12 post-initiation of treatment. The reduced Ba concentration may be the concentration in the blood, serum, plasma, urine, or a combination thereof. The reduced Ba concentration may be in the plasma.

The sC5ab9 concentration may be reduced by at least 40% by week 3 post-initiation of treatment. The sC5ab9 concentration may be reduced by at least 70% by week 6 post-initiation of treatment. The sC5ab9 concentration may be reduced by at least 50% by week 3 post-initiation of treatment. The reduced sC5ab9 concentration may be the concentration in the blood, serum, plasma, urine, or a combination thereof. The sC5ab9 concentration may be in the plasma. The sC5ab9 concentration may be in the urine. The sC5ab9 concentration may be in the plasma and urine.

In the methods described herein, the method may comprise administering the inhibitor of complement to the subject in an amount and with a frequency sufficient to effect a physiological change in the biomarkers.

The physiological change in the biomarker protein(s) may occur within two days, three days, four days, five days, six days, one week, two weeks, three weeks, four weeks, six weeks, two months, nine weeks, or three months or more after administration (e.g., chronic administration) of the inhibitor.

After administration of an effective amount of the complement inhibitor, the concentration of the biomarker protein(s) may be reduced by at least 5%, 10%, 15%, 20%, 25%, 30%, 35%, 40%, 45%, 50%, 55%, 60%, 65%, or 70% following administration of the inhibitor.

After administration of an effective amount of the complement inhibitor, the concentration of the biomarker protein(s) may be reduced to within 50%, 49%, 48%, 47%, 46%, 45%, 44%, 43%, 42%, 41%, 40%, 39%, 38%, 37%, 36%, 35%, 34%, 33%, 32%, 31%, 30%, 29%, 28%, 27%, 26%, 25%, 24%, 23%, 22%, 21%, 20%, 19%, 18%, 17%, 16%, 15%, 14%, 13%, 12%, 11%, 10%, 9%, 8%, 7%, 6%, 5%, 4%, 3%, 2%, or 1% of the normal concentration of the biomarker protein following administration of one or more doses of the complement inhibitor.

The subject of the methods described herein may have received dialysis at least once (e.g., at least twice, thrice, four times, or five times or more) within the three months (e.g., 11, 10, 9, 8, 7, 6, 5, 4, 3, 2, or 1 week(s)) prior to treatment with the complement inhibitor. For example, the subject may have received dialysis one time two months before receiving the complement inhibitor therapy. In another example, the subject may be one who has received dialysis three times within the three month period just prior to receiving the complement inhibitor therapy. Further, relative to the concentrations (e.g., the blood, plasma, serum, and/or urine concentrations) in a healthy human, the concentrations of Ba, sC5b9, cystatin C, and/or creatinine may be elevated.

The subject may be experiencing a first acute complement-mediated thrombotic microangiopathy (CM-TMA) manifestation. For example, prior to treatment with the complement inhibitor, the subject can have elevated concentrations, relative to the normal concentrations, of at least one of the above biomarkers or the above biomarker signature, comprising, e.g., a plurality of the biomarkers.

The subject is one having CM-TMA, but deemed to be in clinical remission (e.g., the subject is one having normal levels of platelets or other hematologic markers such as LDH or haptoglobin). A subject may be one having elevated levels of one or more of the CM-TMA biomarkers described herein including, but not limited, one or more of Ba, sC5b9, cystatin C, and/or creatinine.

The methods described herein may further comprise monitoring the status of the biomarkers and determining whether to start a second therapy (in addition to complement inhibitor therapy) or modify the dosing regimen of one or more second therapies being administered to a complement-mediated thrombotic microangiopathy (CM-TMA) patient. For example, during treatment (e.g., chronic treatment) with a complement inhibitor, the concentration of one or more CM-TMA associated biomarker proteins can be measured in one or more biological fluids obtained from the subject. If the concentration of one or more of the biomarker proteins has not normalized and/or remains elevated, a medical practitioner may elect to administer to the subject one or more additional secondary agents (e.g., anti-inflammatories) to address any pathophysiological effects resulting from the elevated biomarkers.

A normal control concentration, as used in any of the methods described herein, can be (or can be based on), e.g., the concentration of a given biomarker protein in a biological sample or biological samples obtained from one or more (e.g., two, three, four, five, six, seven, eight, nine, 10, 15, 20, 25, 30, 35, or 40 or more) healthy individuals. In some embodiments, a normal control concentration of a biomarker can be (or can be based on), e.g., the concentration of the biomarker in a pooled sample obtained from two or more (e.g., two, three, four, five, six, seven, eight, nine, 10, 15, 20, 25, 30, 35, or 40 or more) healthy individuals. In some embodiments of any of the methods described herein, the pooled samples can be from healthy individuals or, at least, individuals who do not have or are not suspected of having (nor at risk for developing) CM-TMA. For example, determining whether a subject is one having CM-TMA can involve comparing the measured concentration of one or more complement component proteins in a biological sample (or several different types of biological samples) obtained from the patient and comparing the measured concentration to the average concentration of the same proteins in the pooled healthy samples. Such healthy human control concentrations can be, in some embodiments, a range of values, or a median or mean value obtained from the range.

### Administration of Therapeutic Agents

The compositions (e.g., complement inhibitors and/or secondary agents) can be administered to a subject, e.g., a human subject, using a variety of methods that depend, in part, on the route of administration. The route can be, e.g., intravenous injection or infusion (IV), subcutaneous injection (SC), intraperitoneal (IP) injection, or intramuscular injection.

Administration can be achieved by, e.g., local infusion, injection, or by means of an implant. The implant can be of a porous, non-porous, or gelatinous material, including membranes, such as sialastic membranes, or fibers. The implant can be configured for sustained or periodic release of the composition to the subject. See, e.g., U.S. Patent Application Publication No. 2008/0241223; U.S. Pat. Nos. 5,501,856; 4,863,457; and 3,710,795; and EP Patent Nos. EP488401 and EP430539. The composition can be delivered to the subject by way of an implantable device based on, e.g., diffusive, erodible or convective systems, e.g., osmotic pumps, biodegradable implants, electrodiffusion systems, electroosmosis systems, vapor pressure pumps, electrolytic pumps, effervescent pumps, piezoelectric pumps, erosion-based systems, or electromechanical systems.

A suitable dose of a complement inhibitor (e.g., an anti-C5 antibody or fragment thereof), which dose is capable of treating or preventing complement-mediated thrombotic microangiopathy (CM-TMA) in a subject, can depend on a variety of factors including, e.g., the age, sex, and weight of a subject to be treated and the particular inhibitor compound used. For example, a different dose of an siRNA specific for human C5 may be required to treat a subject with CM-TMA as compared to the dose of an anti-C5 antibody required to treat the same patient. Other factors affecting the dose administered to the subject include, e.g., the type or severity of the CM-TMA. For example, a subject having CFH-associated atypical hemolytic uremic syndrome (aHUS) may require administration of a different dosage of the inhibitor than a subject with MCP-associated aHUS. Other factors can include, e.g., other medical disorders concurrently or previously affecting the subject, the general health of the subject, the genetic disposition of the subject, diet, time of administration, rate of excretion, drug combination, and any other additional therapeutics that are administered to the subject. It should also be understood that a specific dosage and treatment regimen for any particular subject will depend upon the judgment of the treating medical practitioner (e.g., doctor or nurse).

The inhibitor can be administered as a fixed dose, or in a milligram per kilogram “mg/kg” dose. The dose can also be chosen to reduce or avoid production of antibodies or other host immune responses against one or more active agents in the composition. Exemplary dosages of an inhibitor, such as an anti-C5 antibody, include, e.g., 1-100 mg/kg, 0.5-50 mg/kg, 0.1-100 mg/kg, 0.5-25 mg/kg, 1-20 mg/kg, and 1-10 mg/kg of body weight.

A human can be intravenously administered an anti-C5 antibody (e.g., eculizumab, ravulizumab) at a dose of about 900 mg about every 12 (e.g., about every 10, 11, 13, 14, 15, 16, 17, 18, 19, 20, 21, 28, 30, 42, or 49 or more) days. See, e.g., Hill et al. (2005) Blood 106(7):2559.

A human can be intravenously administered an anti-C5 antibody (e.g., eculizumab, ravulizumab) at a dose of about 600 (e.g., about 625, 650, 700, 725, 750, 800, 825, 850, 875, 900, 925, 950, or 1,000 or more) mg every week, optionally, for two or more (e.g., three, four, five, six, seven, or eight or more) weeks. Following the initial treatment, the human can be administered the antibody at a dose of about 900 mg about every 14 (e.g., about every 11, 12, 13, 14, 15, 16, 17, 18, 19, 20, 21, 28, 30, 42, or 49 or more) days, e.g., as a maintenance dose. See, e.g., Hillmen et al. (2004) N Engl J Med. 350(6):552-9 and Dmytrijuk et al. (2008) The Oncologist 13(9):993.

A human can be intravenously administered an anti-C5 antibody (e.g., eculizumab, ravulizumab) at a dose of about 900 (e.g., 925, 950, 975, 1000, 1100, or 1200 or more) mg every week, optionally, for two or more (e.g., three, four, five, six, seven, or eight or more) weeks. Following the initial treatment, the human can be administered the antibody at a dose of about 1200 mg about every 14 (e.g., about every 11, 12, 13, 14, 15, 16, 17, 18, 19, 20, 21, 28, 30, 42, or 49 or more) days, e.g., as a maintenance dose. See, e.g., International patent application publication no. WO 2010/054403.

In some embodiments, the disclosure relates to treating CM-TMA in a human subject, comprising, diagnosing or prognosticating CM-TMA in accordance with the foregoing methods, e.g., detection of at least 1, at least 2, at least 3, at least 4, at least 5, at least 6 biomarkers selected from (1) cystatin C; (2) cystatin C/creatinine; (3) complement factor Ba; (4) complement factor Ba/creatinine; (5) complement sC5b-9; and/or (6) complement sC5b-9/creatinine, optionally together with (7) sTNFR1, (8) VCAM-1 and/or (9) thrombomodulin, in the subject's sample; preferably detecting levels of a biomarker signature comprising at least two biomarkers comprising Ba and sC5b9 in a fluid biological sample obtained from the subject; and administering an anti-C5 antibody or an antigen-binding fragment thereof to the subject. In some embodiments, the treatment method comprises detection of the biomarker(s) before and after administration of the anti-C5 antibody or the antigen-binding fragment, wherein, a modulation (e.g., attenuation) in the concentration of the biomarker in the subject's sample after administration of the anti-C5 antibody or the antigen-binding fragment, compared to the concentration thereof prior to administration of the anti-C5 antibody or the antigen-binding fragment indicates that the subject is undergoing effective treatment of CM-TMA.

In some embodiments, the anti-C5 antibody is selected from eculizumab, ravulizumab, tesidolumab, pozelimab, or crovalimab, or a biosimilar thereof or the antigen-binding fragment. For example, preferred biosimilars of eculizumab include, e.g., ABP 959, SB12 or Elizaria. In some embodiments, the anti-C5 antibody comprises eculizumab (SOLIRIS), which is described in WO1995029697 and U.S. Pat. No. 6,355,245 and the heavy and light chains of eculizumab are provided in WO2007106585 and U.S. Pat. No. 9,718,880. In some embodiments, the anti-C5 antibody comprises ravulizumab (also known as ULTOMIRIS®, BNJ441 and ALXN1210), which is described in WO2015134894 and U.S. Pat. No. 9,079,949. In some embodiments, the anti-C5 antibody comprises crovalimab, the antibody sequences being disclosed in Fukuzawa et al. (Sci. Rep., 7:1080, 2017). In some embodiments, the anti-C5 antibody comprises pozelimab, which is described in U.S. Pat. No. 10,633,434. In some embodiments, the anti-C5 antibody comprises tesidolumab (LFG316).

In some embodiments, in the treatment of CM-TMA, the anti-C5 antibody, or antigen binding fragment is administered at a fixed dose. For example, in one embodiment, the anti-C5 antibody, or antigen binding fragment is administered at a dose of 10 mg, 20 mg, 25 mg, 50 mg, 75 mg, 100 mg, 125 mg, 150 mg, 175 mg, 200 mg, 225 mg, 250 mg, 275 mg, 300 mg, 325 mg, 350 mg, 375 mg, 400 mg, 425 mg, 450 mg, 475 mg, 500 mg, 525 mg, 550 mg, 575 mg, 600 mg, 625 mg, 650 mg, 675 mg, 700 mg, 725 mg, 750 mg, 775 mg, 800 mg, 825 mg, 850 mg, 875 mg, 900 mg, 925 mg, 950 mg, 975 mg, 1000 mg, 1100 mg, 1200 mg, 1300 mg, 1400 mg, 1500 mg, 1600 mg, 1700 mg, 1800 mg, 1900 mg, 2000 mg, 2100 mg, 2200 mg, 2300 mg, 2400 mg, 2500 mg, 2600 mg, 2700 mg, 2800 mg, 2900 mg, 3000 mg, 3100 mg, 3200 mg, 3300 mg, 3400 mg, 3500 mg, 3600 mg, 3700 mg, 3800 mg, 3900 mg, 4000 mg, 4100 mg, 4200 mg, 4300 mg, 4400 mg, 4500 mg, 4600 mg, 4700 mg, 4800 mg, 4900 mg, 5000 mg, 5100 mg, 5200 mg, 5300 mg, 5400 mg, 5500 mg, 5600 mg, 5700 mg, 5800 mg, 5900 mg, 6000 mg, 6100 mg, 6200 mg, 6300 mg, 6400 mg, 6500 mg, 6600 mg, 6700 mg, 6800 mg, 6900 mg, 7000 mg, 7100 mg, 7200 mg, 7300 mg, 7400 mg, 7500 mg, 7600 mg, 7700 mg, 7800 mg, 7900 mg, 8000 mg, 8100 mg, 8200 mg, 8300 mg, 8400 mg, 8500 mg, 8600 mg, 8700 mg, 8800 mg, 8900 mg, 9000 mg, 9100 mg, 9200 mg, 9300 mg, 9400 mg, 9500 mg, 9600 mg, 9700 mg, 9800 mg, 9900 mg, 10000 mg, 10100 mg, 10200 mg, 10300 mg, 10400 mg, 10500 mg, 10600 mg, 10700 mg, 10800 mg, 10900 mg, or 11000 mg, without regard to the patient's weight.

In another embodiment, the anti-C5 antibody, or antigen binding fragment thereof, (e.g., ravulizumab (ULTOMIRIS®)) is administered at a dose of 1200 mg, 2400 mg, 2700 mg, 3000 mg, 3300 mg, or 3600 mg.

In another embodiment, the dose of the anti-C5 antibody, or antigen binding fragment is based on the weight of the patient. For example, in one embodiment, 10 mg, 20 mg, 25 mg, 50 mg, 75 mg, 100 mg, 125 mg, 150 mg, 175 mg, 200 mg, 225 mg, 250 mg, 275 mg, 300 mg, 325 mg, 350 mg, 375 mg, 400 mg, 425 mg, 450 mg, 475 mg, 500 mg, 525 mg, 550 mg, 575 mg, 600 mg, 625 mg, 650 mg, 675 mg, 700 mg, 725 mg, 750 mg, 775 mg, 800 mg, 825 mg, 850 mg, 875 mg, 900 mg, 925 mg, 950 mg, 975 mg, 1000 mg, 1100 mg, 1200 mg, 1300 mg, 1400 mg, 1500 mg, 1600 mg, 1700 mg, 1800 mg, 1900 mg, 2000 mg, 2100 mg, 2200 mg, 2300 mg, 2400 mg, 2500 mg, 2600 mg, 2700 mg, 2800 mg, 2900 mg, 3000 mg, 3100 mg, 3200 mg, 3300 mg, 3400 mg, 3500 mg, 3600 mg, 3700 mg, 3800 mg, 3900 mg, 4000 mg, 4100 mg, 4200 mg, 4300 mg, 4400 mg, 4500 mg, 4600 mg, 4700 mg, 4800 mg, 4900 mg, 5000 mg, 5100 mg, 5200 mg, 5300 mg, 5400 mg, 5500 mg, 5600 mg, 5700 mg, 5800 mg, 5900 mg, 6000 mg, 6100 mg, 6200 mg, 6300 mg, 6400 mg, 6500 mg, 6600 mg, 6700 mg, 6800 mg, 6900 mg, 7000 mg, 7100 mg, 7200 mg, 7300 mg, 7400 mg, 7500 mg, 7600 mg, 7700 mg, 7800 mg, 7900 mg, 8000 mg, 8100 mg, 8200 mg, 8300 mg, 8400 mg, 8500 mg, 8600 mg, 8700 mg, 8800 mg, 8900 mg, 9000 mg, 9100 mg, 9200 mg, 9300 mg, 9400 mg, 9500 mg, 9600 mg, 9700 mg, 9800 mg, 9900 mg, 10000 mg, 10100 mg, 10200 mg, 10300 mg, 10400 mg, 10500 mg, 10600 mg, 10700 mg, 10800 mg, 10900 mg, or 11000 mg of the anti-C5 antibody, or antigen binding fragment is administered to a patient weighing ≥30 to <40 kg. In another embodiment, 1200 mg or 2700 mg of the anti-C5 antibody, or antigen binding fragment thereof, (e.g., ravulizumab (ULTOMIRIS®)) is administered to a patient weighing ≥30 to <40 kg.

In another embodiment, 10 mg, 20 mg, 25 mg, 50 mg, 75 mg, 100 mg, 125 mg, 150 mg, 175 mg, 200 mg, 225 mg, 250 mg, 275 mg, 300 mg, 325 mg, 350 mg, 375 mg, 400 mg, 425 mg, 450 mg, 475 mg, 500 mg, 525 mg, 550 mg, 575 mg, 600 mg, 625 mg, 650 mg, 675 mg, 700 mg, 725 mg, 750 mg, 775 mg, 800 mg, 825 mg, 850 mg, 875 mg, 900 mg, 925 mg, 950 mg, 975 mg, 1000 mg, 1100 mg, 1200 mg, 1300 mg, 1400 mg, 1500 mg, 1600 mg, 1700 mg, 1800 mg, 1900 mg, 2000 mg, 2100 mg, 2200 mg, 2300 mg, 2400 mg, 2500 mg, 2600 mg, 2700 mg, 2800 mg, 2900 mg, 3000 mg, 3100 mg, 3200 mg, 3300 mg, 3400 mg, 3500 mg, 3600 mg, 3700 mg, 3800 mg, 3900 mg, 4000 mg, 4100 mg, 4200 mg, 4300 mg, 4400 mg, 4500 mg, 4600 mg, 4700 mg, 4800 mg, 4900 mg, 5000 mg, 5100 mg, 5200 mg, 5300 mg, 5400 mg, 5500 mg, 5600 mg, 5700 mg, 5800 mg, 5900 mg, 6000 mg, 6100 mg, 6200 mg, 6300 mg, 6400 mg, 6500 mg, 6600 mg, 6700 mg, 6800 mg, 6900 mg, 7000 mg, 7100 mg, 7200 mg, 7300 mg, 7400 mg, 7500 mg, 7600 mg, 7700 mg, 7800 mg, 7900 mg, 8000 mg, 8100 mg, 8200 mg, 8300 mg, 8400 mg, 8500 mg, 8600 mg, 8700 mg, 8800 mg, 8900 mg, 9000 mg, 9100 mg, 9200 mg, 9300 mg, 9400 mg, 9500 mg, 9600 mg, 9700 mg, 9800 mg, 9900 mg, 10000 mg, 10100 mg, 10200 mg, 10300 mg, 10400 mg, 10500 mg, 10600 mg, 10700 mg, 10800 mg, 10900 mg, or 11000 mg of the anti-C5 antibody, or antigen binding fragment is administered to a patient weighing ≥40 to <60 kg. In another embodiment, 2400 mg or 3000 mg of the anti-C5 antibody, or antigen binding fragment thereof, (e.g., ravulizumab (ULTOMIRIS®)) is administered to a patient weighing ≥40 to <60 kg.

In another embodiment, 10 mg, 20 mg, 25 mg, 50 mg, 75 mg, 100 mg, 125 mg, 150 mg, 175 mg, 200 mg, 225 mg, 250 mg, 275 mg, 300 mg, 325 mg, 350 mg, 375 mg, 400 mg, 425 mg, 450 mg, 475 mg, 500 mg, 525 mg, 550 mg, 575 mg, 600 mg, 625 mg, 650 mg, 675 mg, 700 mg, 725 mg, 750 mg, 775 mg, 800 mg, 825 mg, 850 mg, 875 mg, 900 mg, 925 mg, 950 mg, 975 mg, 1000 mg, 1100 mg, 1200 mg, 1300 mg, 1400 mg, 1500 mg, 1600 mg, 1700 mg, 1800 mg, 1900 mg, 2000 mg, 2100 mg, 2200 mg, 2300 mg, 2400 mg, 2500 mg, 2600 mg, 2700 mg, 2800 mg, 2900 mg, 3000 mg, 3100 mg, 3200 mg, 3300 mg, 3400 mg, 3500 mg, 3600 mg, 3700 mg, 3800 mg, 3900 mg, 4000 mg, 4100 mg, 4200 mg, 4300 mg, 4400 mg, 4500 mg, 4600 mg, 4700 mg, 4800 mg, 4900 mg, 5000 mg, 5100 mg, 5200 mg, 5300 mg, 5400 mg, 5500 mg, 5600 mg, 5700 mg, 5800 mg, 5900 mg, 6000 mg, 6100 mg, 6200 mg, 6300 mg, 6400 mg, 6500 mg, 6600 mg, 6700 mg, 6800 mg, 6900 mg, 7000 mg, 7100 mg, 7200 mg, 7300 mg, 7400 mg, 7500 mg, 7600 mg, 7700 mg, 7800 mg, 7900 mg, 8000 mg, 8100 mg, 8200 mg, 8300 mg, 8400 mg, 8500 mg, 8600 mg, 8700 mg, 8800 mg, 8900 mg, 9000 mg, 9100 mg, 9200 mg, 9300 mg, 9400 mg, 9500 mg, 9600 mg, 9700 mg, 9800 mg, 9900 mg, 10000 mg, 10100 mg, 10200 mg, 10300 mg, 10400 mg, 10500 mg, 10600 mg, 10700 mg, 10800 mg, 10900 mg, or 11000 mg of the anti-C5 antibody, or antigen binding fragment thereof, is administered to a patient weighing ≥60 to <100 kg. In another embodiment, 2700 mg or 3300 mg of the anti-C5 antibody, or antigen binding fragment thereof, (e.g., ravulizumab (ULTOMIRIS®)) is administered to a patient weighing ≥60 to <100 kg.

In another embodiment, 10 mg, 20 mg, 25 mg, 50 mg, 75 mg, 100 mg, 125 mg, 150 mg, 175 mg, 200 mg, 225 mg, 250 mg, 275 mg, 300 mg, 325 mg, 350 mg, 375 mg, 400 mg, 425 mg, 450 mg, 475 mg, 500 mg, 525 mg, 550 mg, 575 mg, 600 mg, 625 mg, 650 mg, 675 mg, 700 mg, 725 mg, 750 mg, 775 mg, 800 mg, 825 mg, 850 mg, 875 mg, 900 mg, 925 mg, 950 mg, 975 mg, 1000 mg, 1100 mg, 1200 mg, 1300 mg, 1400 mg, 1500 mg, 1600 mg, 1700 mg, 1800 mg, 1900 mg, 2000 mg, 2100 mg, 2200 mg, 2300 mg, 2400 mg, 2500 mg, 2600 mg, 2700 mg, 2800 mg, 2900 mg, 3000 mg, 3100 mg, 3200 mg, 3300 mg, 3400 mg, 3500 mg, 3600 mg, 3700 mg, 3800 mg, 3900 mg, 4000 mg, 4100 mg, 4200 mg, 4300 mg, 4400 mg, 4500 mg, 4600 mg, 4700 mg, 4800 mg, 4900 mg, 5000 mg, 5100 mg, 5200 mg, 5300 mg, 5400 mg, 5500 mg, 5600 mg, 5700 mg, 5800 mg, 5900 mg, 6000 mg, 6100 mg, 6200 mg, 6300 mg, 6400 mg, 6500 mg, 6600 mg, 6700 mg, 6800 mg, 6900 mg, 7000 mg, 7100 mg, 7200 mg, 7300 mg, 7400 mg, 7500 mg, 7600 mg, 7700 mg, 7800 mg, 7900 mg, 8000 mg, 8100 mg, 8200 mg, 8300 mg, 8400 mg, 8500 mg, 8600 mg, 8700 mg, 8800 mg, 8900 mg, 9000 mg, 9100 mg, 9200 mg, 9300 mg, 9400 mg, 9500 mg, 9600 mg, 9700 mg, 9800 mg, 9900 mg, 10000 mg, 10100 mg, 10200 mg, 10300 mg, 10400 mg, 10500 mg, 10600 mg, 10700 mg, 10800 mg, 10900 mg, or 11000 mg of the anti-C5 antibody, or antigen binding fragment, is administered to a patient weighing ≥100 kg. In another embodiment, 3000 mg or 3600 mg of the anti-C5 antibody, or antigen binding fragment thereof, (e.g., ravulizumab (ULTOMIRIS®)) is administered to a patient weighing ≥100 kg.

In another embodiment, a method of treating a human patient with CM-TMA is provided, the method comprising administering to the patient an anti-C5 antibody, or antigen binding fragment thereof (e.g., ravulizumab (ULTOMIRIS®):
(a) once on Day 1 at a dose of: 1200 mg to a patient weighing ≥30 to <40 kg, 2400 mg to a patient weighing ≥40 to <60 kg, 2700 mg to a patient weighing ≥60 to <100 kg, or 3000 mg to a patient weighing ≥100 kg; and
(b) on Day 15 and every eight weeks thereafter at a dose of 2700 mg to a patient weighing ≥30 to <40 kg, 3000 mg to a patient weighing ≥40 to <60 kg, 3300 mg to a patient weighing ≥60 to <100 kg, or 3600 mg to a patient weighing ≥100 kg.

In another embodiment, a method of treating a human patient with CM-TMA is provided, wherein the anti-C5 antibody, or antigen binding fragment thereof, (e.g., ravulizumab (ULTOMIRIS®)) is administered to a patient weighing ≥30 to <40 kg:
(a) once on Day 1 at a dose of 1200 mg; and
(b) on Day 15 and every eight weeks thereafter at a dose of 2700 mg.

In another embodiment, a method of treating a human patient with CM-TMA is provided, wherein the anti-C5 antibody, or antigen binding fragment thereof, (e.g., ravulizumab (ULTOMIRIS®)) is administered to a patient weighing ≥40 to <60 kg:
(a) once on Day 1 at a dose of 2400 mg; and
(b) on Day 15 and every eight weeks thereafter at a dose of 3000 mg.

In another embodiment, a method of treating a human patient with CM-TMA is provided, wherein the anti-C5 antibody, or antigen binding fragment thereof, (e.g., ravulizumab (ULTOMIRIS®)) is administered to a patient weighing ≥60 to <100 kg:
(a) once on Day 1 at a dose of 2700 mg; and
(b) on Day 15 of the administration cycle and every eight weeks thereafter at a dose of 3300 mg.

In another embodiment, a method of treating a human patient with CM-TMA is provided, wherein the anti-C5 antibody, or antigen binding fragment thereof, (e.g., ravulizumab (ULTOMIRIS®)) is administered to a patient weighing ≥100 kg:
(a) once on Day 1 at a dose of 3000 mg; and
(b) on Day 15 and every eight weeks thereafter at a dose of 3600 mg.

In another embodiment, a method of treating a human patient with CM-TMA is provided, the method comprising administering to the patient an effective amount of an anti-C5 antibody, or antigen binding fragment thereof, wherein the anti-C5 antibody, or antigen binding fragment thereof, comprises CDR1, CDR2 and CDR3 heavy chain sequences as set forth in SEQ ID NOs:19, 18 and 3, respectively, and CDR1, CDR2 and CDR3 light chain sequences as set forth in SEQ ID NOs:4, 5 and 6, respectively, and a variant human Fc region that binds to human neonatal Fc receptor (FcRn), wherein the variant human Fc CH3 region comprises Met-429-Leu and Asn-435-Ser substitutions at residues corresponding to methionine 428 and asparagine 434 of a native human IgG Fc region, each in EU numbering, and wherein the anti-C5 antibody, or antigen binding fragment thereof, is administered to the patient
(a) once on Day 1 at a dose of: 1200 mg to a patient weighing ≥30 to <40 kg, 2400 mg to a patient weighing ≥40 to <60 kg, 2700 mg to a patient weighing ≥60 to <100 kg, or 3000 mg to a patient weighing ≥100 kg; and
(b) on Day 15 and every eight weeks thereafter at a dose of 2700 mg to a patient weighing ≥30 to <40 kg, 3000 mg to a patient weighing ≥40 to <60 kg, 3300 mg to a patient weighing ≥60 to <100 kg, or 3600 mg to a patient weighing ≥100 kg.

In another embodiment, the anti-C5 antibody, or antigen binding fragment, is administered at a milligram per kilogram (mg/kg) dose. For example, in one embodiment, the anti-C5 antibody, or antigen binding fragment thereof, is administered at a dose of 0.1 mg/kg, 0.25 mg/kg, 0.5 mg/kg, 0.75 mg/kg, 1.0 mg/kg, 1.25 mg/kg, 1.50 mg/kg, 1.75 mg/kg, 2.0 mg/kg, 2.25 mg/kg, 2.50 mg/kg, 2.75 mg/kg, 3.0 mg/kg, 3.25 mg/kg, 3.50 mg/kg, 3.75 mg/kg, 4.0 mg/kg, 4.25 mg/kg, 4.50 mg/kg, 4.75 mg/kg, 5.0 mg/kg, 5.25 mg/kg, 5.50 mg/kg, 5.75 mg/kg, 6.0 mg/kg, 6.25 mg/kg, 6.50 mg/kg, 6.75 mg/kg, 7.0 mg/kg, 7.25 mg/kg, 7.50 mg/kg, 7.75 mg/kg, 8.0 mg/kg, 8.25 mg/kg, 8.50 mg/kg, 8.75 mg/kg, 9.0 mg/kg, 9.25 mg/kg, 9.50 mg/kg, 9.75 mg/kg, 10.0 mg/kg, 11.25 mg/kg, 11.50 mg/kg, 11.75 mg/kg, 12.0 mg/kg, 12.25 mg/kg, 12.50 mg/kg, 12.75 mg/kg, 13.0 mg/kg, 13.25 mg/kg, 13.50 mg/kg, 13.75 mg/kg, 14.0 mg/kg, 14.25 mg/kg, 14.50 mg/kg, 14.75 mg/kg, 15.0 mg/kg, 15.25 mg/kg, 15.50 mg/kg, 15.75 mg/kg, 16.0 mg/kg, 16.25 mg/kg, 16.50 mg/kg, 16.75 mg/kg, 17.0 mg/kg, 17.25 mg/kg, 17.50 mg/kg, 17.75 mg/kg, 18.0 mg/kg, 18.25 mg/kg, 18.50 mg/kg, 18.75 mg/kg, 19.0 mg/kg, 19.25 mg/kg, 19.50 mg/kg, 19.75 mg/kg, 20.0 mg/kg, 20.25 mg/kg, 20.50 mg/kg, 20.75 mg/kg, 21.0 mg/kg, 21.25 mg/kg, 21.50 mg/kg, 21.75 mg/kg, 22.0 mg/kg, 22.25 mg/kg, 22.50 mg/kg, 22.75 mg/kg, 23.0 mg/kg, 23.25 mg/kg, 23.50 mg/kg, 23.75 mg/kg, 24.0 mg/kg, 24.25 mg/kg, 24.50 mg/kg, 24.75 mg/kg, or 25.0 mg/kg.

In one embodiment, the anti-C5 antibody, or antigen binding fragment is administered once per week, twice per week, three times per week, four times per week, five times per week, six times per week, or daily. In another embodiment, anti-C5 antibody, or antigen binding fragment, is administered once every two weeks, once every three weeks, once every four weeks, once every five weeks, once every six weeks, once every seven weeks, once every eight weeks, once every nine weeks, once every ten weeks, once every eleven weeks, or once every twelve weeks. In another embodiment, the anti-C5 antibody, or antigen binding fragment, is administered at a loading dose on Day 1, followed by a different maintenance dose on Day 15 and every eight weeks thereafter.

In another embodiment, the anti-C5 antibody, or antigen binding fragment thereof, is administered for one or more administration cycles. In one embodiment, the administration cycle is 26 weeks. In another embodiment, the treatment comprises at least 1, 2, 3, 4, 5, 6, 7, 8, 9, 10, or 11 cycles. In another embodiment, the patient is treated for about 1, 2, 3, 4, 5, or 6 months. In another embodiment, the treatment continues for the lifetime of the human patient.

The anti-C5 antibody, or antigen binding fragment, can be administered via any suitable means. In one embodiment, the anti-C5 antibody, or antigen binding fragment (e.g., ravulizumab (ULTOMIRIS®)), is administered intravenously. In another embodiment, the anti-C5 antibody, or antigen binding fragment, is administered subcutaneously.

The patients treated according to the methods described herein can have been vaccinated against meningococcal infections within 3 years prior to, or at the time of, initiating treatment. The patients who received treatment less than 2 weeks after receiving a meningococcal vaccine can also treated with appropriate prophylactic antibiotics until 2 weeks after vaccination. The patients treated according to the methods described herein can be vaccinated against meningococcal serotypes A, C, Y, W135, and/or B.

The patients treated according to the methods described herein can have TMA associated with lupus nephritis, systemic sclerosis, or solid organ transplant. These patients can be vaccinated against Haemophilus influenzae type b (Hib) and Streptococcus pneumoniae prior to treatment.

The treatment regimens described herein are sufficient to maintain particular serum trough concentrations of the anti-C5 antibody, or antigen binding fragment thereof. For example, in one embodiment, the treatment can maintain a serum trough concentration of the anti-C5 antibody, or antigen binding fragment thereof, of 50, 55, 60, 65, 70, 75, 80, 85, 90, 95, 100, 105, 110, 115, 120, 125, 130, 135, 140, 145, 150, 155, 160, 165, 170, 175, 180, 185, 190, 200, 205, 210, 215, 220, 225, 230, 240, 245, 250, 255, 260, 265, 270, 280, 290, 300, 305, 310, 315, 320, 325, 330, 335, 340, 345, 350, 355, 360, 365, 370, 375, 380, 385, 390, 395, or 400 μg/mL or greater. In one embodiment, the treatment maintains a serum trough concentration of the anti-C5 antibody, or antigen binding fragment thereof, of 100 μg/mL or greater. In another embodiment, the treatment maintains a serum trough concentration of the anti-C5 antibody, or antigen binding fragment thereof, of 150 μg/mL or greater. In another embodiment, the treatment maintains a serum trough concentration of the anti-C5 antibody, or antigen binding fragment thereof, of 200 μg/mL or greater. In another embodiment, the treatment maintains a serum trough concentration of the anti-C5 antibody, or antigen binding fragment thereof, of 250 μg/mL or greater. In another embodiment, the treatment maintains a serum trough concentration of the anti-C5 antibody, or antigen binding fragment thereof, of 300 μg/mL or greater. In another embodiment, the treatment maintains a serum trough concentration of the anti-C5 antibody, or antigen binding fragment thereof, of between 100 μg/ml and 200 μg/mL. In another embodiment, the treatment maintains a serum trough concentration of the anti-C5 antibody, or antigen binding fragment thereof, of about 175 μg/mL.

To obtain an effective response, the anti-C5 antibody can be administered to the patient in an amount and with a frequency to maintain at least 50 μg, 55 μg, 60 μg, 65 μg, 70 μg, 75 μg, 80 μg, 85 μg, 90 μg, 95 μg, 100 μg, 105 μg, 110 μg, 115 μg, 120 μg, 125 μg, 130 μg, 135 μg, 140 μg, 145 μg, 150 μg, 155 μg, 160 μg, 165 μg, 170 μg, 175 μg, 180 μg, 185 μg, 190 μg, 195 μg, 200 μg, 205 μg, 210 μg, 215 μg, 220 μg, 225 μg, 230 μg, 235 μg, 240 μg, 245 μg, 250 μg, 255 μg, or 260 μg of antibody per milliliter of the patient's blood. In another embodiment, the anti-C5 antibody is administered to the patient in an amount and with a frequency to maintain between 50 μg and 250 μg of antibody per milliliter of the patient's blood. In another embodiment, the anti-C5 antibody is administered to the patient in an amount and with a frequency to maintain between 100 μg and 200 μg of antibody per milliliter of the patient's blood. In another embodiment, the anti-C5 antibody is administered to the patient in an amount and with a frequency to maintain about 175 μg of antibody per milliliter of the patient's blood.

In another embodiment, to obtain an effective response, the anti-C5 antibody is administered to the patient in an amount and with a frequency to maintain a minimum free C5 concentration. For example, in one embodiment, the anti-C5 antibody can be administered to the patient in an amount and with a frequency to maintain a free C5 concentration of 0.2 μg/mL, 0.3 μg/mL, 0.4 μg/mL, 0.5 μg/mL or below. In another embodiment, the treatment described herein reduces free C5 concentration by greater than 99% throughout the treatment period.

“Chronically administered,” “chronic treatment,” “treating chronically,” or similar grammatical variations thereof refer broadly to a treatment regimen that is employed to maintain a certain threshold concentration of a therapeutic agent in the blood of a patient in order to completely or substantially suppress systemic complement activity in the patient over a prolonged period of time. Accordingly, a subject chronically treated with a complement inhibitor can be treated for a period of time that is greater than or equal to 2 weeks (e.g., 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, or 52 weeks; 1, 2, 3, 4, 5, 6, 7, 8, 9, 10, 11, or 12 months; or 1, 1.5, 2, 2.5, 3, 3.5, 4, 4.5, 5, 5.5, 6, 6.5, 7, 7.5, 8, 8.5, 9, 9.5, 10, 10.5, or 12 years or for the remainder of the patient's life) with the inhibitor in an amount and with a dosing frequency that are sufficient to maintain a concentration of the inhibitor in the patient's blood that inhibits or substantially inhibits systemic complement activity in the patient. The complement inhibitor can be chronically administered to a patient in need thereof in an amount and with a frequency that are effective to maintain serum hemolytic activity at less than or equal to 20 (e.g., 19, 18, 17, 16, 15, 14, 13, 12, 11, 10, 9, 8, 7, 6, or 5) %. See, e.g., Hill et al. (2005) Blood 106(7):2559. The complement inhibitor can be administered to a patient in an amount and with a frequency that are effective to maintain serum lactate dehydrogenase (LDH) levels at within at least 20 (e.g., 19, 18, 17, 16, 15, 14, 13, 12, 11, 10, 9, 8, 7, 6, or 5) % of the normal range for LDH. See Hill et al. (2005) supra. The complement inhibitor may be administered to the patient in an amount and with a frequency that are effective to maintain a serum LDH level less than 550 (e.g., less than 540, 530, 520, 510, 500, 490, 480, 470, 460, 450, 440, 430, 420, 410, 400, 390, 380, 370, 360, 350, 340, 330, 320, 310, 300, 290, 280, or less than 270) IU/L. To maintain systemic complement inhibition in a patient, the complement inhibitor can be chronically administered to the patient, e.g., once a week, once every two weeks, twice a week, once a day, once a month, or once every three weeks.

In some embodiments, the therapy comprises administration of ravulizumab under its dosing schedule. In some embodiments, ravulizumab is administered as an intravenous (i.v.) formulation comprising a single loading dose on Day 1, followed by regular maintenance dosing beginning on Day 15, based on the subject's weight, wherein (a) for a subject whose weight is ≥40 to <60 kilograms (kg), treatment comprises 2400 milligrams (mg) loading, then 3000 mg maintenance dose every 8 weeks; (b) for a subject whose weight is ≥60 to <100 kg, treatment comprises 2700 mg loading, then 3300 mg maintenance dose every 8 weeks; and (c) for a subject whose weight is ≥100 kg, treatment comprises 3000 mg loading, then 3600 mg maintenance dose every 8 weeks. In some embodiments, ravulizumab is administered as a subcutaneous (SC) formulation.

### Compositions

A composition may comprise a therapeutically effective amount of an inhibitor of human complement component C5 (e.g., an anti-C5 antibody or antigen-binding fragment thereof). Two preferred anti-C5 antibodies include but are not limited to eculizumab (SOLIRIS®) and ravulizumab (ULTOMIRIS®). A preferred anti-C5 antigen binding fragment is pexelizumab.

The composition may be a pharmaceutical composition further comprising an excipient, carrier, diluent, stabilizer, buffer, antioxidant, or a combination thereof. A pharmaceutical composition may comprise a therapeutically effective amount of an inhibitor of human complement component C5 (e.g., an anti-C5 antibody or antigen-binding fragment thereof). Two preferred anti-C5 antibodies include but are not limited to eculizumab (SOLIRIS®) and ravulizumab (ULTOMIRIS®). A preferred anti-C5 antigen binding fragment is pexelizumab.

Effective amounts can be readily determined by one of ordinary skill in the art based, in part, on the effect of the administered inhibitor, or the combinatorial effect of the antibody and one or more additional active agents, if more than one agent is used. A therapeutically effective amount of an inhibitor of human complement component C5 (e.g., an anti-C5 antibody) [e.g., eculizumab, ravulizumab] can also vary according to factors such as the disease state, age, sex, and weight of the individual, and the ability of the antibody (and one or more additional active agents) to elicit a desired response in the individual, e.g., amelioration of at least one condition parameter, e.g., amelioration of at least one symptom of CM-TMA. For example, a therapeutically effective amount of an inhibitor of human complement component C5 (e.g., an anti-C5 antibody) can inhibit (lessen the severity of or eliminate the occurrence of) and/or prevent thrombocytopenia, microangiopathic hemolytic anemia, renal failure, and/or any one of the symptoms of CM-TMA known in the art or described herein. Two preferred anti-C5 antibodies include but are not limited to eculizumab (SOLIRIS®) and ravulizumab (ULTOMIRIS®). A therapeutically effective amount is also one in which any toxic or detrimental effects of the composition are outweighed by the therapeutically beneficial effects.

A composition described herein may comprise a therapeutically effective amount of an inhibitor of human complement component C5. Two preferred anti-C5 antibodies include but are not limited to eculizumab (SOLIRIS®) and ravulizumab (ULTOMIRIS®). A composition described herein may comprise a therapeutically effective amount of an antibody, or antigen-binding fragment thereof, which binds to a complement component C5 protein. In some embodiments, the composition may comprise two or more (e.g., three, four, five, six, seven, eight, nine, 10, or 11 or more) different inhibitors of human complement component C5 such that the composition as a whole is therapeutically effective. For example, a composition can may comprise an antibody that binds to a human C5 protein and an siRNA that binds to, and promotes the degradation of, an mRNA encoding a human C5 protein, wherein the antibody and siRNA are each at a concentration that when combined are therapeutically effective. In some embodiments, the composition may comprise the inhibitor and one or more second active agents such that the composition as a whole is therapeutically effective. For example, the composition may comprise an antibody that binds to a human C5 protein and another agent useful for treating or preventing CM-TMA.

Toxicity and therapeutic efficacy of such compositions can be determined by known pharmaceutical procedures in cell cultures or experimental animals (animal models of aHUS). These procedures can be used, e.g., for determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀. Compositions, or inhibitors (e.g., anti-C5 antibodies) [e.g., eculizumab, ravulizumab] of the compositions, that exhibit high therapeutic indices are preferred. While compositions that exhibit toxic side effects may be used, care should be taken to design a delivery system that targets such compounds to the site of affected tissue and to minimize potential damage to normal cells and, thereby, reduce side effects.

The data obtained from the cell culture assays and animal studies can be used in formulating a range of dosage for use in humans. Suitable animal models of aHUS are known in the art and are described in, e.g., Atkinson et al. (2007) Journal of Experimental Medicine 204(6):1245-1248. The dosage of such inhibitors lies generally within a range of circulating concentrations of the inhibitors (e.g., an anti-C5 antibody or antigen-binding fragment thereof) that include the ED₅₀ with little or no toxicity. The dosage may vary within this range depending upon the dosage form employed and the route of administration utilized. For an inhibitor of human complement component C5 (e.g., an anti-C5 antibody) used as described herein (e.g., for treating or preventing CM-TMA), the therapeutically effective dose can be estimated initially from cell culture assays. A dose can be formulated in animal models to achieve a circulating plasma concentration range that includes the IC₅₀ (e.g., the concentration of the test compound which achieves a half-maximal inhibition of symptoms) as determined in cell culture. Such information can be used to more accurately determine useful doses in humans. Levels in plasma may be measured, for example, by high performance liquid chromatography.

The required dose of an inhibitor of human complement component C5 can be determined based on the concentration of human C5 protein in the subject's blood. For example, a subject having a higher concentration of circulating human C5 protein levels may require a higher dose of a human C5 inhibitor than a subject having lower levels of circulating human C5. Methods for determining the concentration of human complement component C5 in a blood-derived fluid sample from a subject are known in the art and described in, e.g., Rawal et al. (1998) J Biol Chem 273(27):16828-16835.

The methods can be performed in conjunction with other therapies for CM-TMA. For example, the composition can be administered to a subject at the same time, prior to, or after, nephrectomy (e.g., bilateral nephrectomy), dialysis, a plasma exchange, or a plasma infusion (see, e.g., Noris et al. (2005) “Non-shiga toxin-associated hemolytic uremic syndrome.” In: Zipfel P (ed). Complement and Kidney Disease. Basel: Birkhauser-Verlag, 65-83).

The inhibitor of human complement component C5 (e.g., an anti-C5 antibody or antigen-binding fragment thereof) [e.g., eculizumab, ravulizumab] can be administered to a subject as a monotherapy. Alternatively, as described above, the inhibitor can be administered to a subject as a combination therapy with another treatment, e.g., another treatment for CM-TMA. For example, the combination therapy can include administering to the subject (e.g., a human patient) one or more additional agents (e.g., anti-hypertensives) that provide a therapeutic benefit to the subject who has, or is at risk of developing, CM-TMA. The inhibitor of human complement component C5 and the one or more additional active agents may be administered at the same time. The inhibitor may be administered first in time and the one or more additional active agents are administered second in time. In some embodiments, the one or more additional active agents are administered first in time and the inhibitor is administered second in time.

The inhibitor of human complement component C5 can replace or augment a previously or currently administered therapy. For example, upon treating with an anti-C5 antibody or antigen-binding fragment thereof, administration of the one or more additional active agents can cease or diminish, e.g., be administered at lower levels. Administration of the previous therapy may be maintained. A previous therapy may be maintained until the level of inhibitor of human C5 reaches a level sufficient to provide a therapeutic effect. The two therapies may be administered in combination.

Monitoring a subject (e.g., a human patient) for an improvement in CM-TMA, as defined herein, means evaluating the subject for a change in a disease parameter, e.g., an improvement in one or more symptoms of the disease. Such symptoms include any of the symptoms of CM-TMA described herein. In some embodiments, the evaluation is performed at least 1 hour, e.g., at least 2, 4, 6, 8, 12, 24, or 48 hours, or at least 1 day, 2 days, 4 days, 10 days, 13 days, 20 days or more, or at least 1 week, 2 weeks, 4 weeks, 10 weeks, 13 weeks, 20 weeks or more, after treatment begins. The subject can be evaluated in one or more of the following periods: prior to beginning of treatment; during the treatment; or after one or more elements of the treatment have been administered. Evaluating may comprise evaluating the need for further treatment, e.g., evaluating whether a dosage, frequency of administration, or duration of treatment should be altered. It may also comprise evaluating the need to add or drop a selected therapeutic modality, e.g., adding or dropping any of the treatments for CM-TMA described herein.

### Concentration of Proteolytic Fragment of Factor B (Ba)

The concentration of the proteolytic fragment of factor B may be measured. The fragment may be Ba. The biological sample can be a blood, serum, plasma, and/or urine sample. The biological sample may be plasma and urine.

The concentration of Ba in the biological sample is deemed elevated when it is at least two fold greater than the normal control concentration of Ba. The concentration of Ba in the biological sample is deemed elevated when it is at least five fold greater than the normal control concentration of Ba. The concentration of Ba in the biological sample is deemed elevated when it is greater than about 1,000 ng/mL. The concentration of Ba in the biological sample is deemed elevated when it is greater than about 1,500 ng/mL. The concentration of Ba in the biological sample is deemed elevated when it is greater than about 2,500 ng/mL.

A post-treatment reduction in Ba concentration (e.g., plasma Ba concentration) of at least 10%, 15%, 20%, 25%, 30%, 35%, 40%, 45%, or 50%, relative to the Ba concentration in a sample of the same type of biological fluid obtained from the subject prior to treatment, indicates that the subject has or is likely to achieve a complete thrombomicroangiopathy (TMA) response (e.g., cessation of TMA events). The reduction may occur by week 12 following the first treatment with the complement inhibitor. The reduction may occur within weeks 12-17 following the first treatment with the complement inhibitor. The reduction may occur by week 26 following the first treatment with the complement inhibitor.

Concentration of Terminal Complement Complex (sC5b-9)

The concentration of sC5b-9 in a sample may be deemed elevated when it is at least ten fold greater than the normal control concentration of sC5b-9. The concentration of sC5b-9 in the biological sample is deemed elevated when it is at least fifty fold greater than the normal control concentration of sC5b-9. The concentration of sC5b-9 in the biological sample is deemed elevated when it is at least one hundred fold greater than the normal control concentration of sC5b-9. The concentration of sC5b-9 in the biological sample is deemed elevated when it is at least 20 ng per mg of urinary creatinine. The concentration of sC5b-9 in the biological sample is deemed elevated when it is at least 30 ng per mg of urinary creatinine. The control concentration may be determined from biological samples from a healthy subject, e.g., a subject who does not have CM-TMA, preferably a subject whose urine sample does not contain detectable levels of sC5b9, as measured via a standard immunoassay.

### Creatinine Levels

In some embodiments of the present disclosure, creatinine levels in the subject's biological samples are measured. Any routine method may be used to measure creatinine levels, including, but not limited to, colorimetric assay (e.g., Jaffe method), enzymatic methods, chemiluminescence assays, chromatographic techniques, molecularly imprinted polymer (MIP), capillary electrophoresis, spectrophotometry methods, potentiometric sensors, electrochemical sensors (ECA), pH meters, and amperometric sensors. These techniques may be performed on blood samples, urine specimens, and even saliva samples, with little to no sample processing (Wei et al., Anal Chem, 2012 Sep. 18; 84(18):7933-7). In some embodiments, the above assays may be improved using nanoparticles (NPs) that bind to creatinine.

Estimated Glomerular Filtration Rate (eGFR)

In adults, a normal GFR value is more than about 90. This may decrease with age, for example, humans aged 70+ may have a GFR above about 75, even in the absence of disease. A GFR value below 60, at any age, is an indication of kidney distress and/or disease. The CKD-EPI Creatinine Equation (2009) may be used to estimate GFR.

Expressed as a single equation:

$$e G F R =$$

$$141 x min {( {S}_{Cr} / κ , 1 )}^{α} x$$

$$max {( {S}_{Cr} / κ , 1 )}^{- 1.209} x$$

$$0.99 {3}^{Age} x$$

$$1.018 [ if female ] x$$

$$1.159 [ if of African descent ]$$

eGFR (estimated glomerular filtration rate)=mL/min/1.73 m²
S_{Cr} (standardized serum creatinine)=mg/dL
κ=0.7 (females) or 0.9 (males)
α=−0.329 (females) or −0.411 (males)
min=indicates the minimum of S_{Cr}/κ or 1
max=indicates the maximum of S_{Cr}/κ or 1
age=years

The CKD-EPI Creatinine Equation (2009) is known in the art and provided by the National Kidney Foundation. National Kidney Foundation website (2021); Levey et al. Ann Intern Med. (2009) 150(9): 604-612.

The status of the biomarkers described herein can be predictive of improvement in the estimated glomerular filtration rate (eGFR) for a CM-TMA patient treated with a complement inhibitor. For example, a reduction in the concentration of Ba and/or sC5b-9 (e.g., within 26 weeks post initial treatment in a chronic treatment regimen) indicates that a CM-TMA patient treated with a complement inhibitor has achieved or is likely to achieve a clinically meaningful improvement in eGFR.

### Urine Protein/Creatinine (Cr) Ratio (UPCR)

A UPCR less than about 0.2 is considered within normal limits. Huang et al. Pediatr Nephrol. (2020) 35(3): 463-468; Karkar & Abdelrahman Saudi J Kidney Dis Transpl (2010) 21: 949-50.

Association Between Biomarkers and Secondary Markers eGFR and/or UPCR

Embodiments of the disclosure relate to use of CM-TMA biomarkers which are associated with the aforementioned secondary markers, e.g., eGFR and/or UPCR, in the diagnosis, management, and treatment of CM-TMA. As is exemplified in the representative Examples section, level of association between secondary markers of CM-TMA, such as eGFR and/or UPCR, with the primary protein biomarkers of the disclosure may be determined using routine techniques, e.g., regression analysis.

### Kits

Also provided are kits comprising various reagents and materials useful for carrying out the methods described herein. The procedures for measuring, diagnosing, evaluating, and/or assessing described herein may be performed by diagnostic laboratories, experimental laboratories, or individual practitioners. The invention provides kits which can be used in any or all of these settings.

Kits may comprise materials and reagents for, among other things, characterizing or processing biological samples (e.g., biological fluids), measuring biomarker levels (e.g., protein or nucleic acid levels), diagnosing CM-TMA in a subject, or monitoring treatment response in a subject according to the methods provided herein. A kit may comprise at least one or more reagents that specifically detect protein levels of one or more CM-TMA biomarker proteins (e.g., Ba, sC5b9, or both) and, optionally, instructions for using the kit. The kit may comprise, e.g., any of the arrays described herein.

The kits may comprise suitable control samples (e.g., biological fluids from normal healthy individuals or a solution comprising a known, control amount of a particular analyte of interest). Kits may comprise instructions for using the kit according to one or more methods described herein and may comprise instructions for processing the biological sample (e.g., a biological fluid) obtained from the subject and/or for performing the test or instructions for interpreting the results.

### EXAMPLES

### Example 1

### Detection of Cm-Tma Biomarkers in Patients

Using data from the phase III study of ravulizumab (terminal C5 complement inhibitor) in adults with aHUS (NCT02949128), baseline (BL; prior to treatment) serum, plasma and urine biomarker levels in patients were compared with levels in healthy volunteers (HV), and evaluated for associations with kidney function (e.g. estimated glomerular filtration rate [eGFR] and urine protein/creatinine [Cr] ratio [UPCR]) at BL and 26 weeks post-treatment initiation. Regression coefficients and p-values (two-sided t-test) are reported.

Results: This analysis included 55 patients: median age 39 (range 19-76) years; 67% female; 53% White, 27% Asian. Specific BL biomarkers were elevated compared with HV, and associations between BL biomarker levels and both BL eGFR and BL UPCR were identified (Table 1). BL plasma complement factor Ba was associated with eGFR changes after 26 weeks of ravulizumab treatment, while urine sC5b-9, sC5b-9/Cr, Ba and Ba/Cr were associated with UPCR changes after 26 weeks of treatment.

TABLE 1

| | Patients | BL biomarker associations | | BL biomarker associations | |
|---|---|---|---|---|---|
| | with | with kidney function | | with kidney function | |
| | elevated | measures (BL) | | measures (26 weeks) | |
| | BL | Regression coefficient | | Regression coefficient | |
| | biomarkers* | (p-value) | | (p-value) | |
| Biomarker | % (n/N) | eGFR | UPCR | eGFR | UPCR |
| Urine cystatin C/Cr | 100.0 | −0.25 | 0.42 | −2.28 | −9.72 |
| (ng/mg Cr) | (36/36) | (0.0002) | (0.0048) | (0.4290) | (0.2385) |
| Plasma Ba | 97.7 | −0.55 | 1.25 | −13.98 | −27.42 |
| (ng/ml) | (42/43) | (<0.0001) | (0.0001) | (0.0085) | (0.1021) |
| Urine cystatin C | 97.6 | −0.25 | 0.51 | −0.62 | −7.38 |
| (ng/ml) | (41/42) | (<0.0001) | (<0.0001) | (0.7788) | (0.2103) |
| Urine Ba | 97.5 | −0.28 | 0.73 | 1.41 | −16.07 |
| (ng/ml) | (39/40) | (<0.0001) | (<0.0001) | (0.5825) | (0.0090) |
| Urine Ba/Cr | 97.1 | −0.31 | 0.77 | 2.40 | −17.28 |
| (ng/mg Cr) | (34/35) | (<0.0001) | (<0.0001) | (0.3957) | (0.0110) |
| Urine sC5b-9** | 88.1 | −0.12 | 0.44 | −0.79 | −16.03 |
| (ng/ml) | (37/42) | (0.0021) | (<0.0001) | (0.5862) | (<0.0001) |
| Urine sC5b-9**/Cr | 88.1 | −0.10 | 0.45 | 1.91 | −21.46 |
| (ng/mg Cr) | (37/42) | (0.0391) | (<0.0001) | (0.3650) | (<0.0001) |
| Plasma sC5b-9 | 79.1 | 0.19 | −0.25 | −3.17 | 15.91 |
| (ng/ml) | (34/43) | (0.1430) | (0.3712) | (0.5269) | (0.1830) |

*compared with observed maximum for HV;
**urine sC5b-9 is undetectable in HV, therefore HV values were set at ½LLOQ (In further studies described herein, the normal donor values for sC5b-9 were set at half of the lower limit of quantification (½LLOQ) for urine sC5b-9).

Conclusions: The complement biomarkers Ba (plasma and urine) and sC5b-9 (urine) were associated with kidney function in patients with aHUS at baseline and over 26 weeks of treatment with anti-C5 therapy. Such biomarkers demonstrate diagnostic potential in CM-TMA and may predict renal response to terminal complement inhibition.

### Example 2

### Detection of Cm-Tma Biomarkers in Patients

aHUS is a form of complement-mediated thrombotic microangiopathy caused by dysregulation of the alternative complement pathway, with or without an identified trigger, typically resulting in kidney damage/failure; damage to other organ systems is common. No validated biomarker/s or assays for the diagnosis or prognosis of aHUS are currently available. Identification of individual, or combinations of, clinically meaningful biomarkers in patients with aHUS, could lead to more rapid diagnosis, prediction of clinical course, and/or earlier treatment initiation.

Using exploratory biomarker data from the phase III study of ravulizumab, a complement C5 inhibitor, in adults with aHUS who were naïve to complement inhibitor therapy, this analysis comprised:
(a) comparing baseline biomarker levels in this population to levels in normal donors;
(b) assessing longitudinal changes in biomarker levels from baseline during treatment with ravulizumab;
(c) examining associations between baseline biomarker levels and both baseline clinical measures and changes in clinical measures after 26 weeks of treatment; and
(d) examining associations between baseline biomarker levels and clinical outcomes after 26 weeks of treatment.

TABLE 2
Patient demographics and disease characteristics

| | |
|---|---|
| | n (%), unless stated, |
| Variable | at baseline (N = 56)* |

Median age at the time of first infusion, yr (range)

| | Age group | 40.1 | (19.5-76.6) |
|---|---|---|---|
| | ≥18 to <30 yr | 11 | (19.6) |
| | ≥30 to <40 yr | 17 | (30.4) |
| | ≥40 to <50 yr | 15 | (26.8) |
| | ≥50 to <60 yr | 5 | (8.9) |
| | ≥60 yr | 8 | (14.3) |
| | Sex, female | 37 | (66.1) |
| Race | | | |
| | Asian | 15 | (26.8) |
| | White | 29 | (51.8) |
| | Undisclosed | 8 | (14.3) |
| | Other | 4 | (7.1) |

Baseline laboratory values, median (range)^{#}

| | | |
|---|---|---|
| Platelets, ×10⁹/L | 95.3 | (18-473) |
| LDH, U/I | 508 | (230-3249) |
| Serum creatinine, μmol/L^{†} | 284 | (51-1027) |
| eGFR, mL/min/1.73 m² | 10 | (4-80) |
| Hemoglobin, g/L | 85 | (60.5-140) |

TABLE 3
Patient demographics and disease characteristics

| | n (%) [95% CI] at |
|---|---|
| Variable | 26 weeks (N = 56)* |
| Complete TMA response | 30 (53.6) [39.6-67.5] |
| Hematologic normalization^{‡} | 41 (73.2) [60.7-85.7] |
| Platelet count normalization | 47 (83.9) [73.4-94] |
| LDH normalization | 43 (76.8) [64.8-88.7] |
| ≥25% improvement in serum creatinine | 33 (58.9) [45.2-72.7] |
| from baseline | |

TABLE 4
Patient demographics and disease characteristics

| Variable | |
|---|---|
| Patients with ≥1 identified pathogenic | n (%), unless stated, |
| variant or autoantibody^{§,¶} | at baseline (N = 56)* |
| C3 | 1 (2.6) |
| Cd46 | 2 (5.1) |
| CFB | 1 (2.6) |
| CFH | 2 (5.1) |
| CFH autoantibody | 2 (5.1) |

See also Rondeau et al. Kidney Int (2020) 97: 1287-96

See also Rondeau er al. Kidney Int (2020) 97: 1287-96

CF, complement factor; CI, confidence interval; eGFR, estimated glomerular filtration rate; LDH, lactate dehydrogenase; TMA, thrombotic microangiopathy

TABLE 5
Most biomarkers were elevated in adults with aHus
before treatment, compared to normal donor levels

| | Biomarker levels at baseline* | | Patients with elevated baseline |
|---|---|---|---|
| | Adults with aHUS | Normal donors | biomarkers compared to observed |
| | n^{#}, median | n^{#}, median | maximum for normal donors |
| Biomarker | (min, max) | (min, max) | n/N (%) |
| Urine cystatin C/Cr (ng/mg Cr) | 36, 9052.1 | 5, 40.8 | 36/36 |
| | (99.9, 38649.5) | (22.1, 42.7) | (100) |
| Urine cystatin C (ng/ml) | 42, 4068.5 | 10, 16.3 | 41/42 |
| | (4.7, 45876.9) | (4.7, 31.1) | (98) |
| Plasma Ba (ng/ml) | 43, 6288.1 | 20, 891.4 | 42/43 |
| | (1280.4, 29886.6) | (684.7, 1375.9) | (98) |
| Urine Ba (ng/ml) | 40, 4129.0 | 10, 3.7 | 39/40 |
| | (5.0, 40200.0) | (1.0, 8.0) | (98) |
| Urine Ba/Cr (ng/mg Cr) | 35, 8358.0 | 10, 4.2 | 34/35 |
| | (22.0, 58478.0) | (3.0, 56.0) | (97) |
| Urine sC5b-9/Cr (ng/mg Cr)^{†} | 32, 321.5 | 10, 5.9 | 31/32 |
| | (9.3, 81600.0) | 3.6, 13.9 | (97) |
| Serum sTNF-RI (pg/mL) | 54,7407.9 | 19, 838.0 | 51/54 |
| | (1330.6, 24781.6) | (658.8, 1617.1) | (94) |
| Plasma thrombomodulin (ng/ml) | 44, 11.3 | 19, 4.3 | 40/44 |
| | (3.8, 27.2) | (3.1, 5.8) | (91) |
| Urine sC5b-9 (ng/ml)^{†} | 42, 130.5 | 10, 2.6 | 37/42 |
| | (2.6, 154000.0) | 2.6, 2.6 | (88) |
| Plasma sC5b-9 (ng/ml) | 43, 937.7 | 10, 423.9 | 34/43 |
| | (325.0, 10007.0) | (349.0, 585.0) | (79) |
| Plasma D-dimer (ng/ml) | 49, 4076.6 | 19, 551.9 | 31/49 |
| | (474.0, 47862.1) | (39.6, 2739.0) | (63) |
| Serum sVCAM-1 (ng/ml) | 54, 1506.0 | 20, 560.0 | 7/54 |
| | (763.5, 5522.2) | (251.3, 3803.2) | (13) |

*A nominal total of 55 adults with aHUS, 10 normal urine donors and 20 normal serum/plasma donors were assessed in this study;
^{#}n refers to the number of patients/donors with evaluable data.
^{†}tsC5b-9 is undetectable in normal donor urine samples, therefore for this analysis normal donor values were set at ½LLOQ for urine sC5b-9.

All bioanalytical assays, both commercial ELISA and custom MSD, were validated to 2018 FDA guidance for biomarker methods. Sample testing and data reporting were performed in regulated, quality-controlled laboratories.

Outlined boxes indicate results for complement-specific biomarkers in plasma, and in urine normalized to creatinine.

aHUS, atypical hemolytic uremic syndrome, Cr, creatinine; ELISA, enzyme-linked immunosorbent assay; FDA, United States Food and Drug Administration; LLOQ, lower limit of quantification; MSD, meso scale discovery

With the exception of serum sVCAM-1, all biomarkers were elevated in 63-100% of adults with aHUS, as compared to the maximum observed levels in normal donors. The complement-specific biomarkers Ba and sC5b-9 were elevated in >75% of patients, with plasma and urine Ba elevated in ≥97%. This is a set of biomarkers examined in urine, plasma and serum.

In urine samples, the inventors also assessed the biomarkers levels normalized against creatinine. Outlined highlight results for complement factor Ba and protein sC5b-9, which are specifically associated with complement activity (of the alternative and terminal pathways, respectively). These results support he use of these biomarkers both in the diagnosis of aHUS and other complement-mediated conditions.

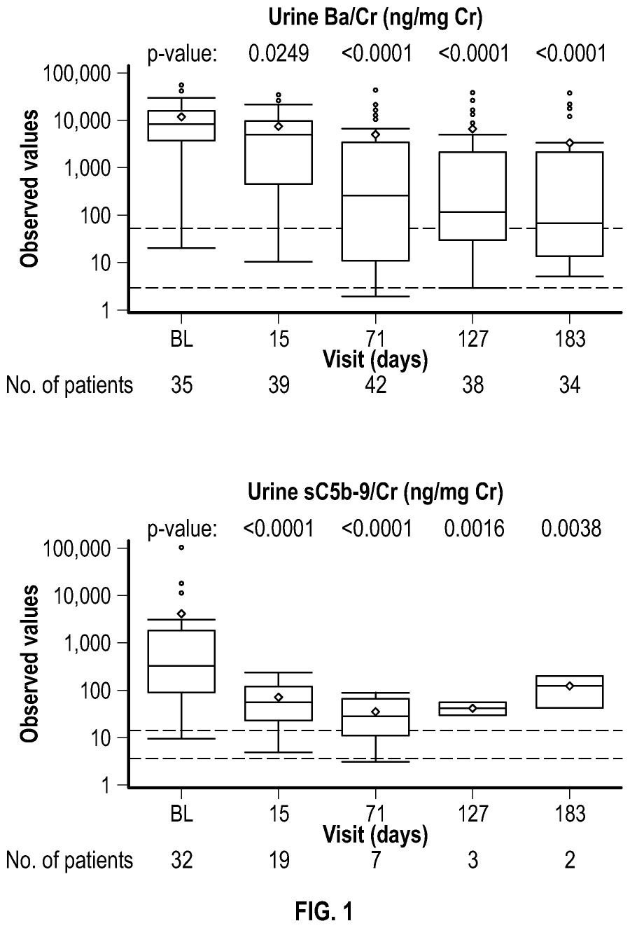

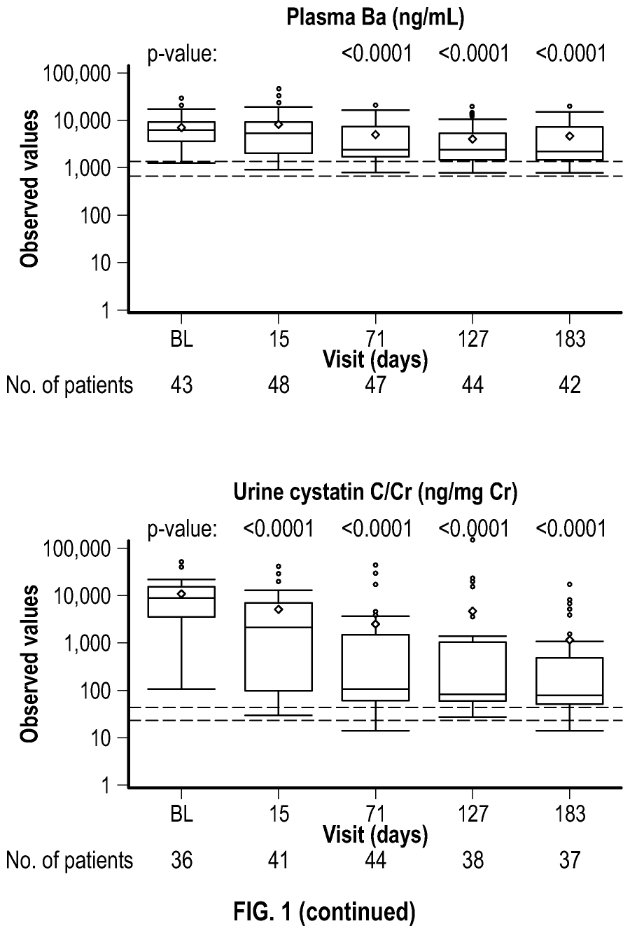

FIG. 1 depicts biomarkers of complement dysregulation and renal damage decrease with Ravulizumab treatment. Urine Ba/Cr and sC5b-9/Cr levels significantly declined after the first dose of ravulizumab, demonstrating an early pharmacodynamic effect of C5 inhibition. Plasma Ba levels were significantly reduced compared to baseline levels from day 71, indicating persistent terminal inhibition can reduce alternative complement pathway activity. Urine cystatin C/Cr levels significantly declined after the first dose of ravulizumab, suggesting renal recovery. The inventors evaluated normalized data, which best demonstrated the change over time. These results show that complement-specific biomarkers do decrease in response to anti-C5 therapy, which supporting the use of treatment response monitoring.

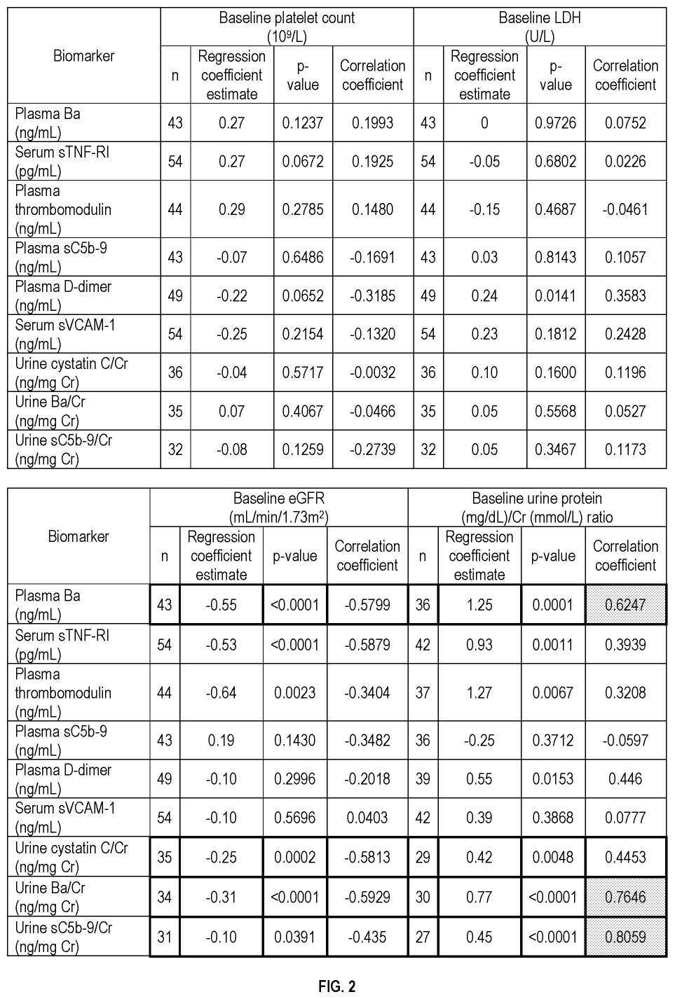

FIG. 2 depicts associations between baseline biomarkers and baseline clinical measures. Baseline levels of plasma Ba and urine Ba/Cr and sC5b-9/Cr, and urine cystatin C/Cr, were significantly associated with lower baseline eGFR and elevated baseline proteinuria. Significant associations were also observed between non-complement-specific biomarkers, and the same clinical measures. The results for Ba and sC5b-9 are highlighted because these complement-specific biomarkers were significantly associated with kidney function measures at baseline, specifically eGFR and proteinuria.

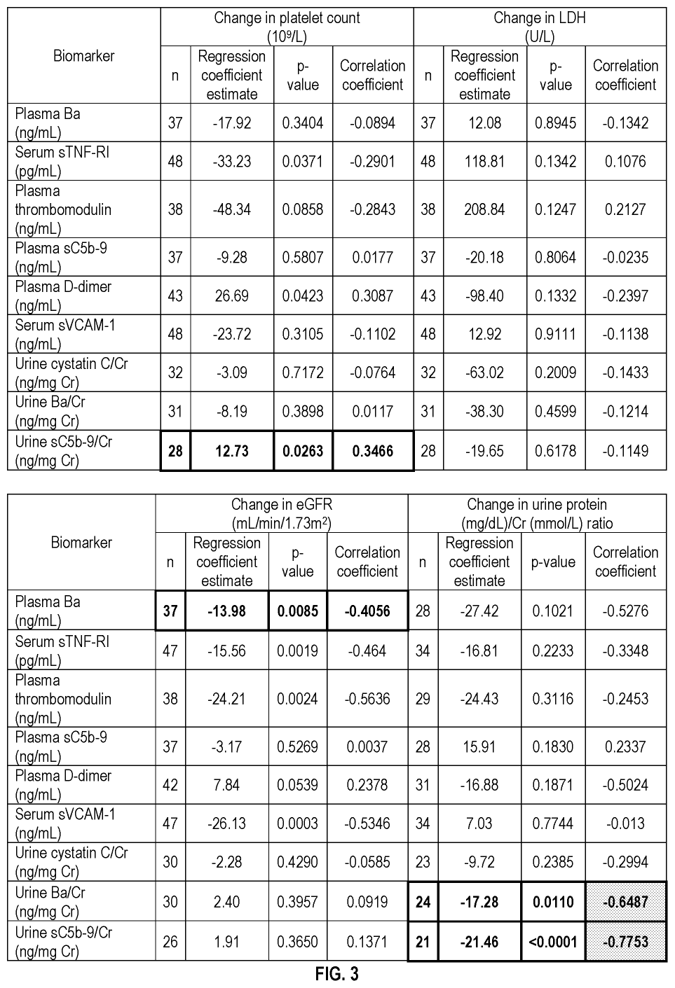

FIG. 3 depicts associations between baseline biomarkers and changes in clinical measures over 26 weeks of treatment. After 26 weeks of treatment with ravulizumab: (a) baseline urine sC5b-9/Cr was significantly associated with platelet count increases; (b) baseline urine Ba/Cr and sC5b-9/Cr were significantly inversely associated with change in proteinuria levels; and (c) baseline plasma Ba was significantly inversely associated with change in eGFR. Some significant associations were also observed for the non-complement-specific biomarkers.

The highlighted results show that baseline Ba in plasma and, particularly, Ba and sC5b-9 in urine (normalized results) were associated with kidney function during C5 inhibitor treatment. This indicates they may be used in in predicting (evaluating) treatment outcomes.

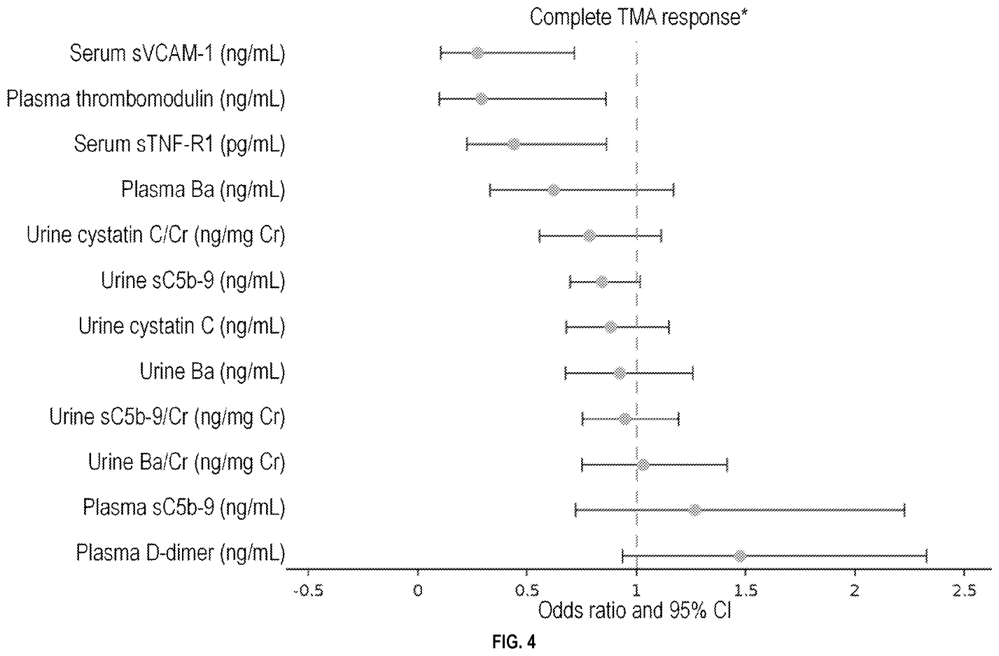

FIG. 4 depicts data showing that baseline biomarker levels were significantly associated with select clinical outcomes at 26 weeks. Odds ratios are derived from a logistic regression analysis with the response variable as the dependent variable and the log of the baseline biomarker level as the independent variable, and represent the increased (or decreased) odds of achieving the efficacy response for every 2-fold increase in baseline biomarker. At 26 weeks: (1) lower baseline levels of serum sVCAM-1 and sTNF-R1 were significantly associated with a higher likelihood of achieving complete TMA response; (2) lower baseline levels of plasma thrombomodulin were significantly associated with a higher likelihood of achieving complete TMA response; and (3) lower baseline levels of urine sC5b-9 approached a significant association with a higher likelihood of achieving complete TMA response. The inventors evaluated the complete TMA response: the combined measure of normalization of platelet count and LDH, along with improvement in serum creatinine. Of the complement-specific biomarkers, sC5b-9 in urine had the most marked association with complete TMA response after C5 inhibitor treatment. Of the non-complement-specific biomarkers, those associated with renal function had the most significant association.

From the evaluation of 7 biomarkers in blood and/or urine for clinical diagnostic, prognostic and predictive utility in newly diagnosed treatment-naïve adults with aHUS, the inventors made the following observations:
(1) Complement biomarkers in plasma (Ba and sC5b-9) and urine (Ba) were elevated in patients with aHUS compared to normal donors;
(2) Baseline levels of plasma Ba, urine Ba/Cr and sC5b-9/Cr, and of urine cystatin C/Cr, were significantly associated with kidney function at baseline (lower eGFR and elevated proteinuria);
(3) Complement biomarker levels (urine Ba/Cr and sC5b-9/Cr) significantly declined with C5 inhibition, demonstrating an early pharmacodynamic effect of C5 inhibition on complement activity; and
(4) Baseline urine sC5b-9 levels approached a significant association with complete TMA response at 26 weeks.

Measuring complement biomarkers Ba and sC5b-9, especially in urine, has utility in the diagnosis of CM-TMA/aHUS, as well as predictive utility in monitoring the responses to complement-inhibitor treatment, e.g., treatment with an anti-C5 antibody such as eculizumab (SOLIRIS®) and/or ravulizumab (ULTOMIRIS®). This may be attributed to their association with kidney function at baseline and 26 weeks. For example, complement-specific biomarkers in urine may be particularly useful in monitoring their renal function.

This data suggests that measuring plasma and urine biomarker proteins can yield a diagnostic, prognostic and/or predictive signature of complement-mediated renal diseases such as CM-TMA and response to complement-inhibitor therapy.

### Example 3

Exploratory Diagnostic and Prognostic Biomarkers of Complement-Mediated Thrombotic Microangiopathy (CM-TMA) in Adults with Atypical Hemolytic Uremic Syndrome (aHUS): Analysis of a Phase 3 Study of Ravulizumab

### INTRODUCTION

TMA is a broad clinical term encompassing conditions presenting as the triad of thrombocytopenia, microangiopathic hemolytic anemia and microvascular thrombosis, typically leading to organ damage/failure. CM-TMA is a subset of TMA disorders driven by the generation of complement activation with or without presence of complement gene mutations and acquired autoantibodies. aHUS, often considered the prototypical form of CM-TMA, is caused by overactivation of the alternative pathway of complement, leading to terminal complement overactivation. The complement cascade, including the production of split products.

Despite information about the pathophysiology of CM-TMA/aHUS, several clinical challenges exist relating to its differential diagnosis and prognosis. One such challenge is a lack of sensitive, specific, and clinically validated biomarkers that can define this heterogenous disease population. Therefore, continued exploration of novel matrices, sample collection protocols, and bioanalytical assays are needed to identify and accurately measure individual and/or combinations of clinically meaningful biomarkers in patients with aHUS. Identification of a biomarker “signature” in patients with aHUS may help inform diagnosis, treatment decisions and therapeutic monitoring of patients with other forms of CM-TMA.

A study from Cofiell et al. explored the pharmacodynamic effect of complement inhibition—via treatment with the anti-complement C5 monoclonal antibody (mAb) eculizumab—on markers of multiple cellular processes in patients with aHUS, including complement activation, inflammation, endothelial damage, thrombosis, and renal injury. Cofiell et al. Eculizumab reduces complement activation, inflammation, endothelial damage, thrombosis, and renal injury markers in aHUS. Blood (2015) 125(21):3253-62.

The study found that, following treatment with eculizumab, the majority of blood and urine markers of these processes returned to normal or near-normal levels when compared to normal donor samples. Id. Conversely, markers of proximal alternative pathway and endothelial activation remained partially elevated following treatment, suggesting on-going dysregulation in pathways upstream of eculizmab's mechanism of action. Id. Nevertheless, biomarker utility was not the key focus of the study from Cofiell et al., meaning more specific assessments of the utility of the analyzed proteins as aHUS/CM-TMA-specific biomarkers are required.

### Methods

The current analysis used data from the phase 3 clinical study of ravulizumab in adults with aHUS to explore the potential diagnostic and prognostic utility of blood and urinary biomarkers of complement activation, inflammation, and renal injury in adults with aHUS. The inventors assessed longitudinal changes in biomarker levels from baseline following ravulizumab treatment for 26 and 52 weeks. The inventors retrospectively assessed data from the initial evaluation (26 weeks) and extension period (52 weeks) of the phase 3 study of ravulizumab in adults with aHUS in a post-hoc analysis.

Biomarkers of interest are plasma complement factor Ba, sC5b-9, thrombomodulin, and D-dimer; serum sTNF-RI and sVCAM-1; urine factor Ba, sC5b-9, and cystatin C. Spot urine collections were performed under exploratory conditions of random time of day and void volume. To control for these variations in urine collection, urine biomarkers were normalized against urine creatinine concentration. Serum was collected using a Becton-Dickinson (BD) SST® vacutainer, plasma for factor Ba, sC5b-9 and thrombomodulin was collected using a BD P100® vacutainer, citrated plasma was collected for D-dimer. Urine was collected in a proprietary cryostabilizing solution with protease inhibitor. All normal donor serum, plasma and urine biomarker samples were generated matrix equivalent to the aHUS biomarker sample matrix. All bioanalytical methods were custom or modified commercial solid-phase ligand-binding immunoassays optimized to minimize preanalytical variability and fully validated to FDA Biomarker guidance including acceptable long-term storage stability that exceeded the time between collection and testing for all clinical samples.

Baseline serum, plasma, and urine biomarker levels in patients with aHUS—assessed prior to treatment initiation—were compared with biomarker levels obtained from normal donor samples; normal serum and plasma samples were obtained from Alexion in-house donors, BioIVT (Westbury, NY, USA), and Sanguine Biosciences (Waltham, MA, USA) while normal urine samples were obtained from Alexion in-house donors and Sanguine Biosciences.

Longitudinal biomarker levels, and changes in levels from baseline following treatment with ravulizumab, were evaluated using a mixed model for repeated measures (MMRM) analysis, with log-transformed biomarker level as the dependent variable, and fixed categorical effect of visit and fixed continuous effect of log-transformed baseline level as a covariate.

Baseline biomarkers were also assessed in the context of plasma exchange (PE)/plasma infusion (PI) and dialysis status at baseline.

Baseline biomarkers were evaluated for associations with key clinical measures of TMA—platelet count, lactate dehydrogenase (LDH) concentration, estimated glomerular filtration rate (eGFR) and urine protein/creatinine ratio (UPCR)—via Spearman correlation coefficients between baseline biomarker levels and baseline clinical measures, and between baseline biomarker levels and change in clinical measures at 26 and 52 weeks from treatment initiation. Simple linear regression analyses were also performed, with both the log-transformed baseline clinical measure as the dependent variable and the log-transformed baseline biomarker level as the independent variable, as well as with the 26—and 52-week changes from baseline in clinical variable as the dependent variable and the log-transformed baseline biomarker level as the independent variable. For associations with baseline clinical measures, every 2-fold increase in baseline biomarker results in an increase (or decrease) in the lab value by a factor of two raised to the regression coefficient. When assessing associations with the change in clinical measures at 26 and 52 weeks, every two-fold increase in baseline biomarker results in the change in lab value increasing (or decreasing) by the regression coefficient. Two-sided t-test was used to test for significance following regression analyses.

Further linear regression analyses were performed for clinical measures (e.g., platelet count, LDH, eGFR and UPCR) based on baseline biomarkers and biomarker levels at week 52. The dependent variable was 52-week change from baseline in clinical measure and the independent variables were the log(2)-transformed baseline biomarker level and log(2)-transformed post-dose biomarker level. Results were stratified by complete TMA response status (defined as: normalization of platelet count, normalization of LDH, and >25% improvement in serum creatinine from baseline, met concurrently at two separate assessments, at least 4 weeks apart) at 52 weeks.

Logistic regression analysis was performed to assess any relationship between identified biomarkers at baseline and complete TMA response. Patients were included in this analysis set if the end point of complete TMA response was confirmed by assessment at 26 weeks. For the logistic regression analysis, clinical response was set as the dependent variable with the log of the baseline biomarker level as the independent variable. Odds ratios (ORs) and 95% confidence intervals (CI) were reported, with ORs representing the increased (or decreased) odds of achieving the efficacy response for every two-fold increase in baseline biomarker; statistical significance following logistic regression analyses was assessed via Wald testing.

Baseline levels of Ba and sC5b-9, in urine and plasma, from aHUS patients and normal donors, were input into CombiROC (an online tool available at CombiROC.eu) to generate receiver operating characteristic (ROC) curves for analysis of sensitivity and specificity. The website is maintained by the Protein Microarray and Bioinformatics Facilities at the National Institute of Molecular Genetics (Milan, Italy).

### Results

### Baseline Demographics

A total of 56 adults with aHUS were enrolled in this clinical trial of which 55 were included in the current biomarker analyses; patient demographics and disease characteristics are outlined in Table 6. The median age at the time of first infusion was 40.1 years, and the majority were female (66%) and white (520%).

TABLE 6
Baseline demographics and disease characteristics

| | n (%), unless | |
|---|---|---|
| | stated, at baseline | |
| Variable | (N = 56)^{a} | |
| Median age at the time of first infusion, yr (range) | 40.1 | (19.5-76.6) |
| Age group | | |
| ≥18 to <30 yr | 11 | (19.6) |
| ≥30 to <40 yr | 17 | (30.4) |
| ≥40 to <50 yr | 15 | (26.8) |
| ≥50 to <60 yr | 5 | (8.9) |
| ≥60 yr | 8 | (14.3) |
| Sex, female | 37 | (66.1) |
| Race | | |
| Asian | 15 | (26.8) |
| White | 29 | (51.8) |
| Undisclosed | 8 | (14.3) |
| Other | 4 | (7.1) |
| Baseline laboratory values, median (range)^{b} | | |
| Platelets, ×10⁹/L | 95.3 | (18-473) |
| LDH, U/I | 508 | (230-3249) |
| Serum creatinine, μmol/L^{c} | 284 | (51-1027) |
| eGFR, mL/min/1.73 m² | 10 | (4-80) |
| Hemoglobin, g/L | 85 | (60.5-140) |
| Patients with ≥1 identified pathogenic | | |
| variant or autoantibody^{d,e} | | |
| C3 | 1 | (2.6) |
| CD46 | 2 | (5.1) |
| CFB | 1 | (2.6) |
| CFH | 2 | (5.1) |
| CFH autoantibody | 2 | (5.1) |

^{a}Full analysis set, of which N = 55 formed the biomarkers analysis set;
^{b}Baseline values may be after plasma exchange/infusion in some patients;
^{c}Safety set, N = 58;
^{‡}Defined as normalization of both LDH and platelet count;
^{d}Number of patients tested, n = 39;
^{e}One additional patient had a CFH autoantibody; however, this patient was not tested for genetics. In total, 3 of 52 patients tested for CFH autoantibodies were positive.
CF, complement factor;
eGFR, estimated glomerular filtration rate;
LDH, lactate dehydrogenase;
TMA, thrombotic microangiopathy;
yr, years
Table reproduced from Rondeau E, et al. Kidney Int 2020; 97: 1287-96, under the terms of the Creative Commons CC-BY-NC-ND license.

Baseline Biomarker Level Comparison Between Patients with aHUS and Normal Donors

A comparison of baseline biomarker levels in adults with aHUS and normal donors is presented in Table 7. All biomarkers were elevated in patients with aHUS compared with normal donors, with the exception of plasma sC5b-9 and serum sVCAM-1. Alternative-complement-pathway-specific biomarker factor Ba, in plasma and urine, was elevated in ≥950% of patients. Terminal-complement-pathway-specific biomarker sC5b-9, in urine, was elevated in >85% of patients.

TABLE 7
Comparison of baseline biomarker levels in adults with aHUS and normal donors

| | | | | Patients with elevated | |
|---|---|---|---|---|---|
| | Biomarker levels at baseline | | | baseline biomarkers | |
| | n^{a}, median (min, max) | | | compared to observed | |
| | Adults with | Normal | | maximum for normal | |
| Biomarker | aHUS^{b} | donors^{b} | p-value | donors n/N (%) | |
| Urine cystatin C/Cr | 36 | 10 | <0.0001 | 35/36 | (97) |
| (ng/mg Cr) | 9052.1 | 32.8 | | | |
| | (99.9, 38649.5) | (18.7, 329.3) | | | |
| Urine Ba/Cr (ng/mg Cr) | 35 | 2 | <0.0001 | 34/35 | (97) |
| | 8358.0 | 3.9 | | | |
| | (22.0, 58478.0) | (1.0, 108.0) | | | |
| Serum sTNF-RI (pg/mL) | 54 | 20 | <0.0001 | 52/54 | (96) |
| | 7407.9 | 867.2 | | | |
| | (1330.6, 24781.6) | (663.4, 1595.1) | | | |
| Plasma Ba (ng/ml) | 43 | 59 | <0.0001 | 41/43 | (95) |
| | 6288.1 | 951.8 | | | |
| | (1280.4, 29886.6) | (586.6, 1784.5) | | | |
| Urine sC5b-9/Cr | 42 | 22 | <0.0001 | 36/42 | (86) |
| (ng/mg Cr)^{c} | 256.0 | 3.8 | | | |
| | (4.1, 1629629.6) | (0.9, 13.9) | | | |
| Plasma thrombomodulin | 44 | 20 | <0.0001 | 35/44 | (80) |
| (ng/ml) | 11.3 | 4.4 | | | |
| | (3.8, 27.2) | (3.2, 5.8) | | | |
| Plasma D-dimer (ng/ml) | 49 | 19 | <0.0001 | 35/49 | (71) |
| | 4076.6 | 462.9 | | | |
| | (474.0 47862.1) | (91.2, 2064.8) | | | |
| Plasma sC5b-9 (ng/ml) | 43 | 39 | <0.0001 | 9/43 | (21) |
| | 937.7 | 406.6 | | | |
| | (325.0, 10007.0) | (216.0, 1505.0) | | | |
| Serum sVCAM-1 | 54 | 20 | <0.0001 | 5/54 | (9) |
| (ng/ml) | 1506.0 | 559.6 | | | |
| | (763.5, 5522.2) | (279.8, 3965.7) | | | |

^{a}n details the number of patients/donors with evaluable data;
^{b}A nominal total of 55 adults with aHUS, 25 normal urine, 20 normal serum and 60 normal plasma donors were assessed in this study.
aHUS, atypical hemolytic uremic syndrome;
Cr, creatinine

Longitudinal Changes in Biomarkers from Baseline Following Treatment with Ravulizumab

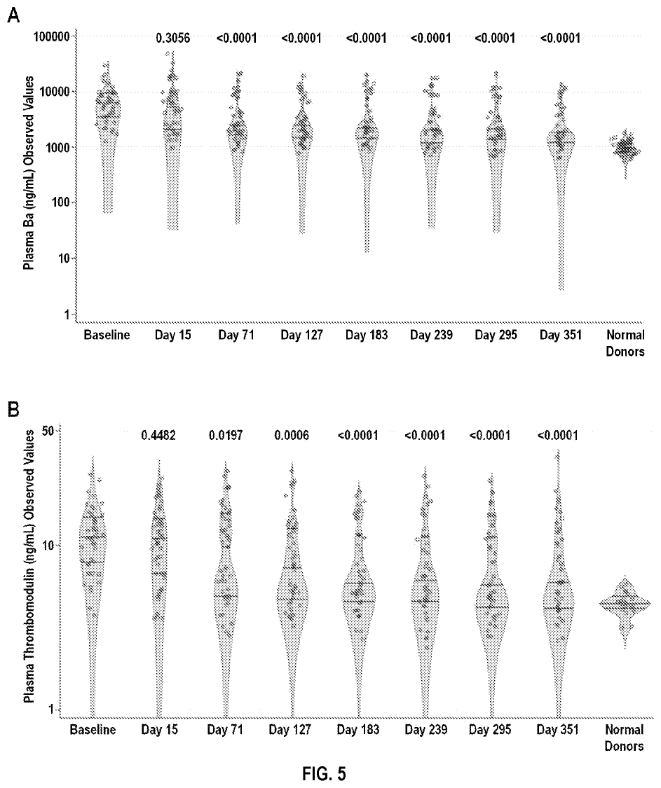

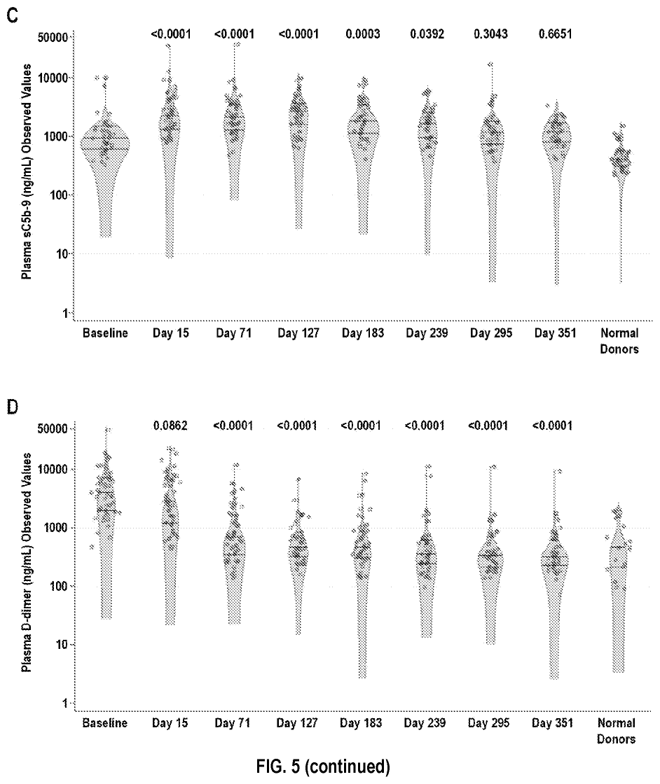

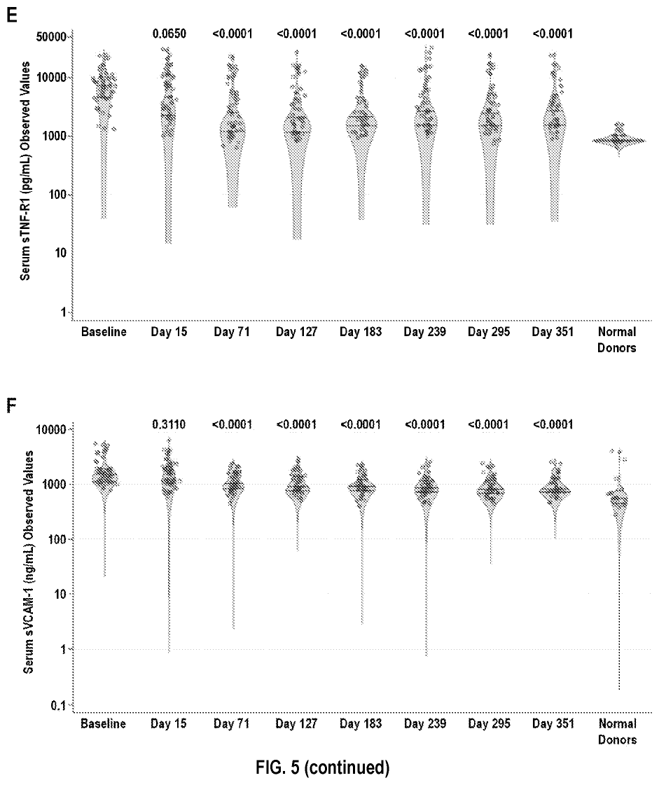

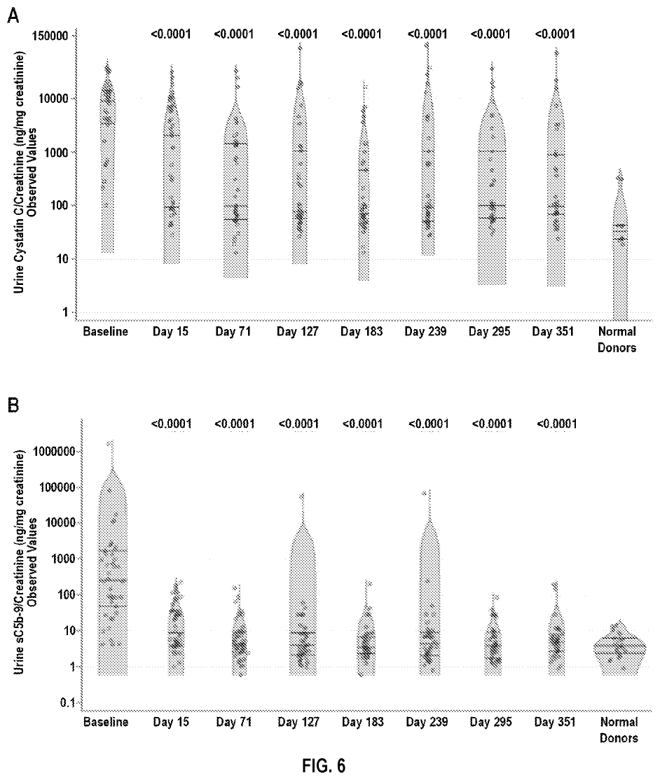

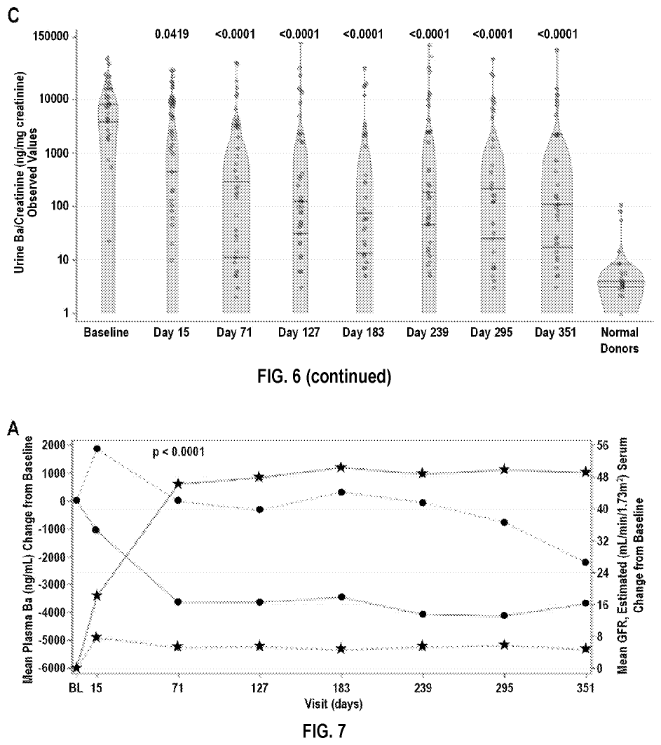

In ravulizumab-treated adults with aHUS, a pharmacodynamic effect was observed for both blood and urine biomarkers, including significant reductions in creatinine normalized urine sC5b-9 and Ba levels compared to baseline at all collection timepoints (FIGS. 5A-F and 6A-C). Serum sTNF-R1 and plasma D-dimer levels also decreased significantly from baseline levels until day 127 (FIG. 5).

Baseline Biomarker Associations with Pre-Treatment Plasma Exchange/Plasma Infusion (PE/P1) and Dialysis

When baseline biomarkers were assessed for any associations with pre-treatment PE/PI status, patients who received PE/PI within 7 days of the first infusion of ravulizumab had significantly lower plasma sC5b-9 levels and higher urine Ba/creatinine levels compared with patients who did not (Table 8). Patients who received dialysis within 5 days of treatment initiation had significantly higher plasma Ba, serum sTNF-R1, and urine Ba/creatinine than patients who did not (Table 9).

TABLE 8
Biomarker levels at baseline by pre-treatment plasma exchange/plasma infusion

| | PE/PI within 7 days | | No PE/PI within 7 days | | |
|---|---|---|---|---|---|
| | of first infusion | | of first infusion | | |
| | N = 36 | | N = 19 | | |
| | | Median | | Median | |
| Biomarker | n | (min, max) | n | (min, max) | p-value |
| Plasma Ba (ng/ml) | 28 | 6677.1 | 15 | 5437.6 | 0.5493 |
| | | (1280.4, 29886.6) | | (2059, 20190.8) | |
| Serum sTNF-R1 (pg/ml) | 36 | 7613.3 | 18 | 6942.8 | 0.3354 |
| | | (1330.6, 24781.6) | | (1367.3, 21815.0) | |
| Plasma | 29 | 11.1 | 15 | 12.3 | 0.4210 |
| thrombomodulin (ng/ml) | | (4.2, 20.2) | | (3.8, 27.2) | |
| Plasma sC5b-9 (ng/ml) | 28 | 784.7 | 15 | 1259.8 | 0.0428 |
| | | (325.0, 10007.0) | | (424.0, 9929.0) | |
| Plasma D-dimer (ng/ml) | 32 | 4714.9 | 17 | 3172.9 | 0.4817 |
| | | (1043.6, 47862.1) | | (474.0, 19258.3) | |
| Serum sVCAM-1 (ng/ml) | 36 | 1435.2 | 18 | 1612.0 | 0.3354 |
| | | (824.3, 5522.2) | | (763.5, 5185.1) | |
| Urine cystatin C/ | 22 | 9052.1 | 14 | 9310.5 | 0.7827 |
| creatinine (ng/mg creatinine) | | (212.3, 38649.5) | | (99.9, 32867.8) | |
| Urine Ba/ | 20 | 9950.5 | 15 | 7521.0 | 0.0597 |
| creatinine (ng/mg creatinine) | | (1829.0, 58478.0) | | (22.0, 22647.0) | |
| Urine sC5b-9/ | 20 | 426.5 | 12 | 318.5 | 0.4958 |
| creatinine (ng/mg creatinine) | | (9.0, 81600.0) | | (22.0, 1840.0) | |

PE, plasma exchange;
PI, plasma infusion

TABLE 9
Biomarker Levels at Baseline by Dialysis Within 5 Days Prior to Treatment Initiation

| | Dialysis within 5 days | | No dialysis within 5 days | | |
|---|---|---|---|---|---|
| | from treatment initiation | | from treatment initiation | | |
| | N = 28 | | N = 27 | | |
| | | Median | | Median | |
| Biomarker | n | (min, max) | n | (min, max) | p-value |
| Plasma Ba (ng/ml) | 24 | 7360.8 | 19 | 3829.0 | 0.0252 |
| | | (1741.6, 29886.6) | | (1280.4, 14232.5) | |
| Serum sTNF-R1 (pg/ml) | 28 | 9042.3 | 26 | 5436.4 | 0.0021 |
| | | (1514.4, 24781.6) | | (1330.6, 13737.3) | |
| Plasma | 25 | 12.1 | 19 | 11.2 | 0.3254 |
| thrombomodulin (ng/ml) | | (4.2, 25.2) | | (3.8, 27.2) | |
| Plasma sC5b-9 (ng/ml) | 24 | 807.9 | 19 | 1259.8 | 0.1092 |
| | | (325.0, 1584.0) | | (424.0, 10007.0) | |
| Plasma D-dimer (ng/ml) | 25 | 5301.7 | 24 | 3303.9 | 0.1585 |
| | | (1528.0, 47862.1) | | (474.0, 19258.3) | |
| Serum sVCAM-1 (ng/ml) | 28 | 1435.2 | 26 | 1612.0 | 0.8694 |
| | | (981.8, 5522.2) | | (763.5, 4050.0) | |
| Urine cystatin C/ | 13 | 10688.8 | 23 | 5486.8 | 0.0561 |
| creatinine (ng/mg creatinine) | | (709.7, 38649.5) | | (100.0, 32867.8) | |
| Urine Ba/ | 13 | 16790.0 | 22 | 6509.5 | 0.0022 |
| creatinine (ng/mg creatinine) | | (3849.0, 58478.0) | | (22.0, 22647.0) | |
| Urine sC5b-9/ | 13 | 957.0 | 19 | 148.0 | 0.0551 |
| creatinine (ng/mg creatinine) | | (31.0, 81600.0) | | (9.0, 2410.0) | |

Baseline Biomarker Associations with Baseline Clinical Measures

Following simple regression analysis of baseline clinical measures against baseline biomarker levels (Table 10), increased plasma Ba, plasma thrombomodulin, and serum sTNF-RI were all significantly associated with lower baseline eGFR values. Normalized urine cystatin C, sC5b-9 and Ba were also associated with lower baseline eGFR values. Increased plasma Ba, thrombomodulin, and D-dimer, serum sTNF-RI, and normalized urine cystatin C, sC5b-9, and Ba were all significantly associated with higher baseline UPCR values. No significant associations between any biomarker levels at baseline and baseline platelet count were identified.

TABLE 10
Comparison of baseline biomarker levels with baseline platelet count, LDH, eGFR and UPCR

| | Baseline platelet count (10⁹/L) | | | | Baseline LDH (U/L) | | | |
|---|---|---|---|---|---|---|---|---|
| | | Regression | | | | Regression | | |
| | | coefficient | P | Correlation | | coefficient | P | Correlation |
| Biomarker | n | estimate | value | coefficient | n | estimate | value | coefficient |
| Plasma Ba (ng/ml) | 43 | 0.27 | 0.1237 | 0.1993 | 43 | 0 | 0.9726 | 0.0752 |
| Plasma thrombomodulin (ng/ml) | 44 | 0.29 | 0.2785 | 0.148 | 44 | −0.15 | 0.4687 | −0.0461 |
| Plasma sC5b-9 (ng/ml) | 43 | −0.07 | 0.6486 | −0.1691 | 43 | 0.03 | 0.8143 | 0.1057 |
| Plasma D-dimer (ng/ml) | 49 | −0.22 | 0.0652 | −0.3185 | 49 | 0.24 | 0.0141 | 0.3583 |
| Serum sTNF-RI (pg/ml) | 54 | 0.27 | 0.0672 | 0.1925 | 54 | −0.05 | 0.6802 | 0.0226 |
| Serum sVCAM-1 (ng/ml) | 54 | −0.25 | 0.2154 | −0.132 | 54 | 0.23 | 0.1812 | 0.2428 |
| Urine cystatin C/creatinine | 36 | −0.04 | 0.5717 | −0.0032 | 36 | 0.1 | 0.16 | 0.1196 |
| (ng/mg creatinine) | | | | | | | | |
| Urine sC5b-9/creatinine | 32 | −0.08 | 0.1259 | −0.2739 | 32 | 0.05 | 0.3467 | 0.1173 |
| (ng/mg creatinine) | | | | | | | | |
| Urine Ba/creatinine | 35 | 0.07 | 0.4067 | −0.0466 | 35 | 0.05 | 0.5568 | 0.0527 |
| (ng/mg creatinine) | | | | | | | | |
| | | | | | Baseline urine protein (mg/dL)/ | | | |
| | Baseline eGFR (mL/min/1.73 m²) | | | | creatinine (mmol/L) ratio | | | |
| | | Regression | | | | Regression | | |
| | | coefficient | P | Correlation | | coefficient | P | Correlation |
| Biomarker | n | estimate | value | coefficient | n | estimate | value | coefficient |
| Plasma Ba (ng/ml) | 43 | −0.55 | <0.0001 | −0.5799 | 36 | 1.25 | 0.0001 | 0.6247 |
| Plasma thrombomodulin (ng/ml) | 44 | −0.64 | 0.0023 | −0.3404 | 37 | 1.27 | 0.0067 | 0.3208 |
| Plasma sC5b-9 (ng/ml) | 43 | 0.19 | 0.143 | 0.3482 | 36 | −0.25 | 0.3712 | −0.0597 |
| Plasma D-dimer (ng/ml) | 49 | −0.1 | 0.2996 | −0.2018 | 39 | 0.55 | 0.0153 | 0.446 |
| Serum sTNF-RI (pg/ml) | 54 | −0.53 | <0.0001 | −0.5879 | 42 | 0.93 | 0.0011 | 0.3939 |
| Serum sVCAM-1 (ng/ml) | 54 | −0.1 | 0.5696 | 0.0403 | 42 | 0.39 | 0.3868 | 0.0777 |
| Urine cystatin C/creatinine | 35 | −0.25 | 0.0002 | −0.5813 | 29 | 0.42 | 0.0048 | 0.4453 |
| (ng/mg creatinine) | | | | | | | | |
| Urine sC5b-9/creatinine | 31 | −0.1 | 0.0391 | −0.435 | 27 | 0.45 | <0.0001 | 0.8059 |
| (ng/mg creatinine) | | | | | | | | |
| Urine Ba/creatinine | 34 | −0.31 | <0.0001 | −0.5929 | 30 | 0.77 | <0.0001 | 0.7646 |
| (ng/mg creatinine) | | | | | | | | |

eGFR, estimated glomerular filtration rate;
LDH, lactate dehydrogenase;
TMA, thrombotic microangiopathy;
yr, years

Baseline Biomarker Associations with Changes in Clinical Measures at 26 and 52 Weeks

Analysis of the change in clinical measures at 26 and 52 weeks using simple regression against baseline biomarker levels found no significant associations between baseline biomarker levels and change in platelet count at either time point (Table 11). Baseline urine sC5b-9/creatinine levels were significantly associated with change in LDH at 26 weeks, but not at 52 weeks (Table 12). Increased plasma Ba and D-dimer, serum sTNF-R1 and sVCAM-1, and urine cystatin C/creatinine and Ba/creatinine were significantly inversely associated with the change from baseline in eGFR at 26 weeks (Table 13).

TABLE 11
Comparison of baseline biomarkers with change in platelet count at 26 and 52 weeks

| | Change in platelet count | | | | Change in platelet count | | | |
|---|---|---|---|---|---|---|---|---|
| | (10⁹/L) at 26 weeks | | | | (10⁹/L) at 52 weeks | | | |
| | | Regression | | | | Regression | | |
| | | coefficient | P | Correlation | | coefficient | P | Correlation |
| Biomarker | n | estimate | value | coefficient | n | estimate | value | coefficient |
| Plasma Ba (ng/ml) | 42 | −18.55 | 0.3866 | −0.1929 | 39 | −32.98 | 0.1029 | −0.3939 |
| Plasma thrombomodulin | 42 | −33.13 | 0.3386 | −0.3377 | 39 | −44.78 | 0.1666 | −0.4324 |
| (ng/ml) | | | | | | | | |
| Plasma sC5b-9 | 42 | 35.51 | 0.0725 | 0.2388 | 39 | 3.47 | 0.9088 | −0.0690 |
| (ng/ml) | | | | | | | | |
| Plasma D-dimer | 47 | −10.32 | 0.3891 | −0.1662 | 44 | −15.87 | 0.2084 | −0.2150 |
| (ng/ml) | | | | | | | | |
| Serum sTNF-R1 | 45 | −2.80 | 0.8630 | −0.2887 | 44 | −14.59 | 0.2794 | −0.3661 |
| (pg/ml) | | | | | | | | |
| Serum sVCAM-1 | 46 | −45.54 | 0.1510 | −0.2698 | 44 | −54.65 | 0.0892 | −0.2832 |
| (ng/ml) | | | | | | | | |
| Urine cystatin C/creatinine | 37 | −11.02 | 0.3402 | −0.3146 | 35 | −10.67 | 0.1216 | −0.3229 |
| (ng/mg creatinine) | | | | | | | | |
| Urine sC5b-9/creatinine | 39 | −13.21 | 0.3564 | −0.1420 | 40 | 1.59 | 0.8894 | 0.1465 |
| (ng/mg creatinine) | | | | | | | | |
| Urine Ba/creatinine | 34 | −9.70 | 0.1571 | −0.3724 | 36 | −7.65 | 0.1859 | −0.3239 |
| (ng/mg creatinine) | | | | | | | | |

TABLE 12
Comparison of baseline biomarkers with change in lactate dehydrogenase at 26 and 52 weeks

| | Change in LDH (U/L) at 26 weeks | | | | Change in LDH (U/L) at 52 weeks | | | |
|---|---|---|---|---|---|---|---|---|
| | | Regression | | | | Regression | | |
| | | coefficient | P | Correlation | | coefficient | | Correlation |
| Biomarker | n | estimate | value | coefficient | n | estimate | P value | coefficient |
| Plasma Ba (ng/ml) | 42 | 195.63 | 0.0799 | 0.1843 | 39 | 148.96 | 0.1987 | 0.1848 |
| Plasma thrombomodulin | 42 | 197.92 | 0.2781 | 0.1623 | 39 | 221.86 | 0.2131 | 0.2826 |
| (ng/ml) | | | | | | | | |
| Plasma sC5b-9 (ng/ml) | 42 | 21.65 | 0.8431 | −0.0523 | 39 | 252.52 | 0.1375 | 0.0051 |
| Plasma D-dimer | 47 | −14.05 | 0.8307 | 0.0661 | 44 | −80.41 | 0.3033 | −0.0499 |
| (ng/ml) | | | | | | | | |
| Serum sTNF-R1 | 45 | 17.80 | 0.8469 | 0.2282 | 44 | 54.70 | 0.5201 | 0.2779 |
| (pg/ml) | | | | | | | | |
| Serum sVCAM-1 | 46 | 102.39 | 0.5688 | 0.1740 | 44 | 148.72 | 0.4569 | 0.1051 |
| (ng/ml) | | | | | | | | |
| Urine cystatin C/creatinine | 37 | 31.73 | 0.6243 | 0.1268 | 35 | 42.99 | 0.4074 | 0.1249 |
| (ng/mg creatinine) | | | | | | | | |
| Urine sC5b-9/creatinine | 39 | 139.35 | 0.0454 | 0.1478 | 40 | 63.14 | 0.3448 | 0.1049 |
| (ng/mg creatinine) | | | | | | | | |
| Urine Ba/creatinine | 34 | 47.37 | 0.1846 | 0.2990 | 36 | 42.76 | 0.2334 | 0.1996 |
| (ng/mg creatinine) | | | | | | | | |

LDH, lactate dehydrogenase

TABLE 13
Comparison of baseline biomarkers with change in estimated glomerular filtration rate at 26 and 52 weeks

| | Change in eGFR (mL/min/1.73 m²) | | | | Change in eGFR (mL/min/1.73 m²) | | | |
|---|---|---|---|---|---|---|---|---|
| | at 26 weeks | | | | at 52 weeks | | | |
| | | Regression | | | | Regression | | |
| | | coefficient | P | Correlation | | coefficient | P | Correlation |
| Biomarker | n | estimate | value | coefficient | n | estimate | value | coefficient |
| Plasma Ba (ng/ml) | 41 | −18.36 | 0.0006 | −0.7447 | 38 | −20.38 | <0.0001 | −0.7381 |
| Plasma thrombomodulin | 41 | −21.78 | 0.0163 | −0.6654 | 38 | −30.15 | <0.0001 | −0.8356 |
| (ng/ml) | | | | | | | | |
| Plasma D-dimer | 41 | 3.55 | 0.5508 | −0.0846 | 38 | −11.99 | 0.1516 | −0.4229 |
| (ng/ml) | | | | | | | | |
| Pasma sC5b-9 | 46 | −13.29 | <0.0001 | −0.4875 | 43 | −15.05 | 0.0001 | −0.5023 |
| (ng/ml) | | | | | | | | |
| Serum sTNF-R1 | 44 | −23.96 | <0.0001 | −0.7938 | 43 | −22.50 | <0.0001 | −0.8260 |
| (pg/ml) | | | | | | | | |
| Serum sVCAM-1 | 45 | −26.37 | 0.0037 | −0.5847 | 43 | −24.93 | 0.0042 | −0.6880 |
| (ng/ml) | | | | | | | | |
| Urine cystatin C/creatinine | 36 | −8.85 | 0.0059 | −0.4938 | 34 | −8.54 | 0.0003 | −0.6691 |
| (ng/mg creatinine) | | | | | | | | |
| Urine sC5b-9/creatinine | 38 | −1.51 | 0.7046 | 0.1018 | 39 | 0.25 | 0.9437 | 0.1678 |
| (ng/mg creatinine) | | | | | | | | |
| Urine Ba/creatinine | 33 | −8.31 | <0.0001 | −0.8692 | 35 | −5.45 | 0.0008 | −0.7028 |
| (ng/mg creatinine) | | | | | | | | |

eGFR, estimated glomerular filtration rate

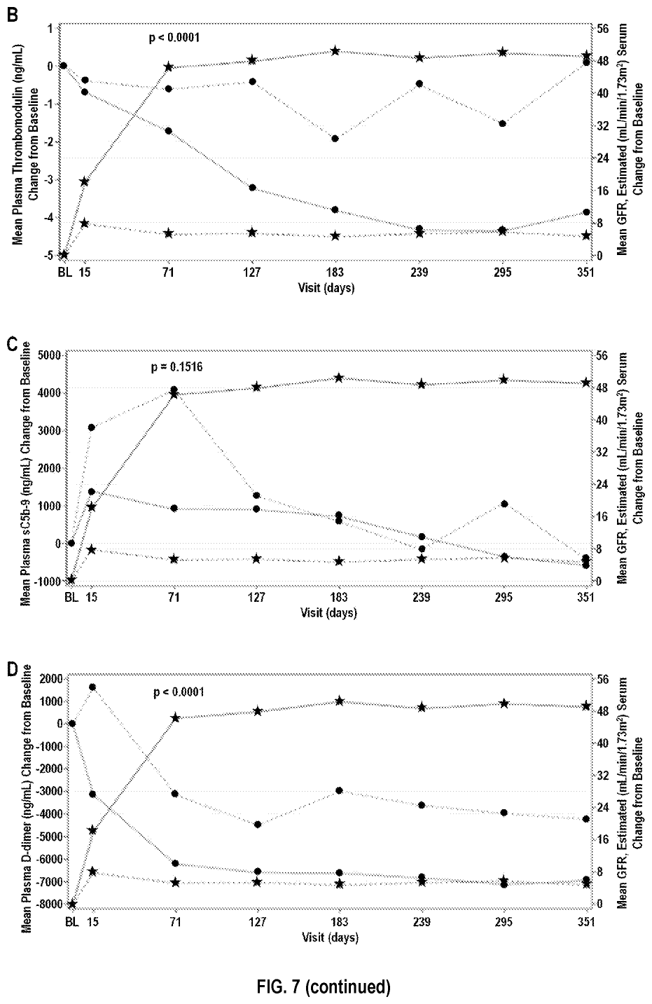

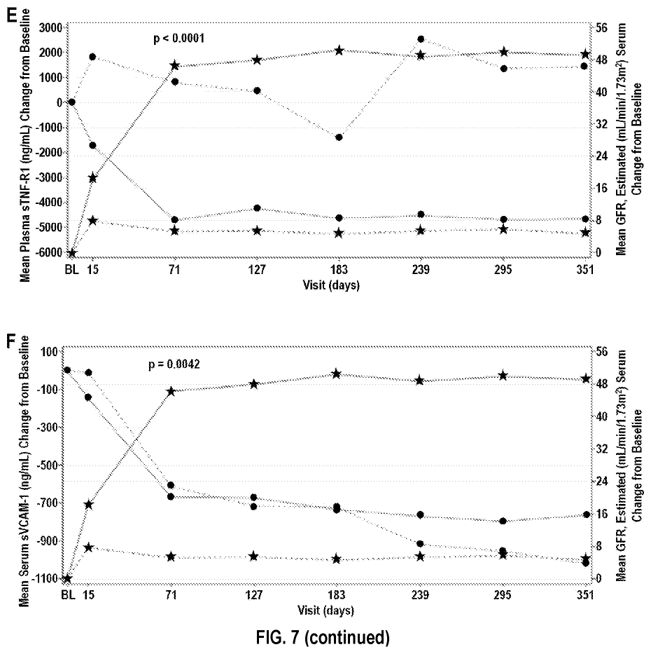

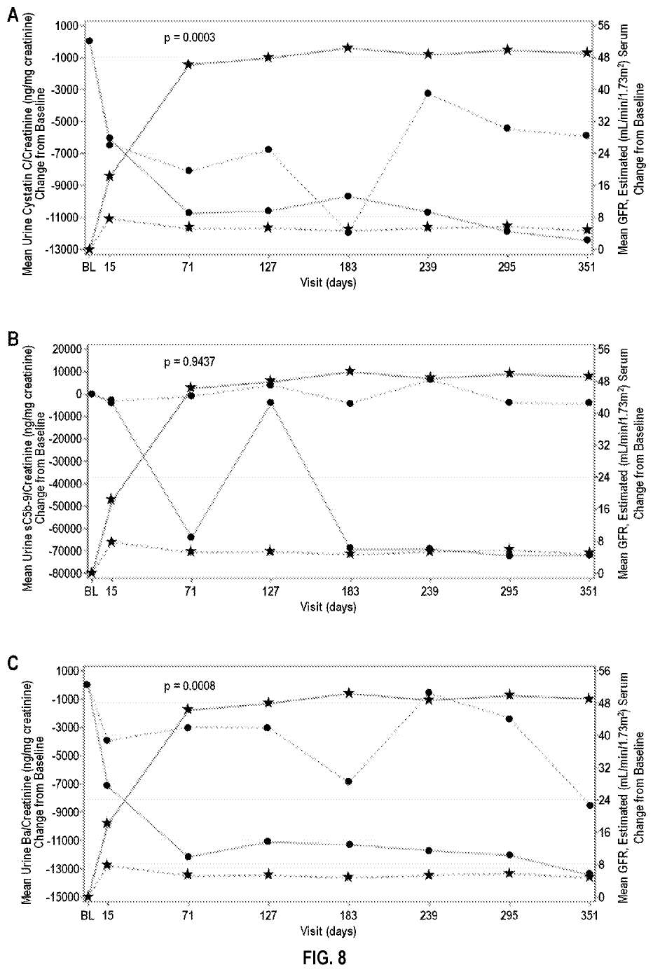

At 52 weeks the same relationships were observed, with the addition of a significant inverse association with baseline plasma thrombomodulin levels (Table 13 and FIGS. 7 and 8).

Baseline Biomarker Associations with Complete TMA Response at 26 and 52 Weeks

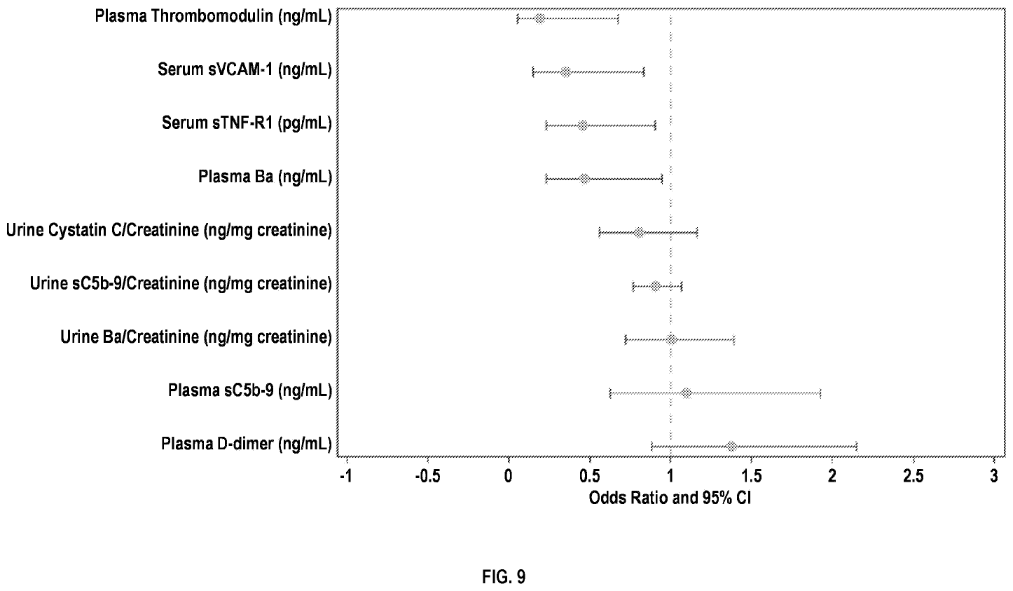

When assessed by logistic regression analysis, increased serum sVCAM-1, plasma thrombomodulin, serum sTNF-R1, and plasma Ba levels were found to be significantly associated with a higher likelihood of achieving complete TMA response; for normalized urine sC5b-9 the association approached significance. The same associations were observed at 52 weeks of treatment (FIG. 9). Complete TMA response was achieved in >60% of patients and median time to complete TMA response was 86 days (95% CI 42, 401).

Baseline Biomarker Levels Associations with Complete TMA Response

Baseline levels of plasma Ba and urine sC5b-9, plasma thrombomodulin, and serum sTNF-R1, were significantly lower in complete TMA responders compared to non-responders (FIG. 9).

### Clinical Sensitivity and Specificity of Select Baseline Biomarker Levels

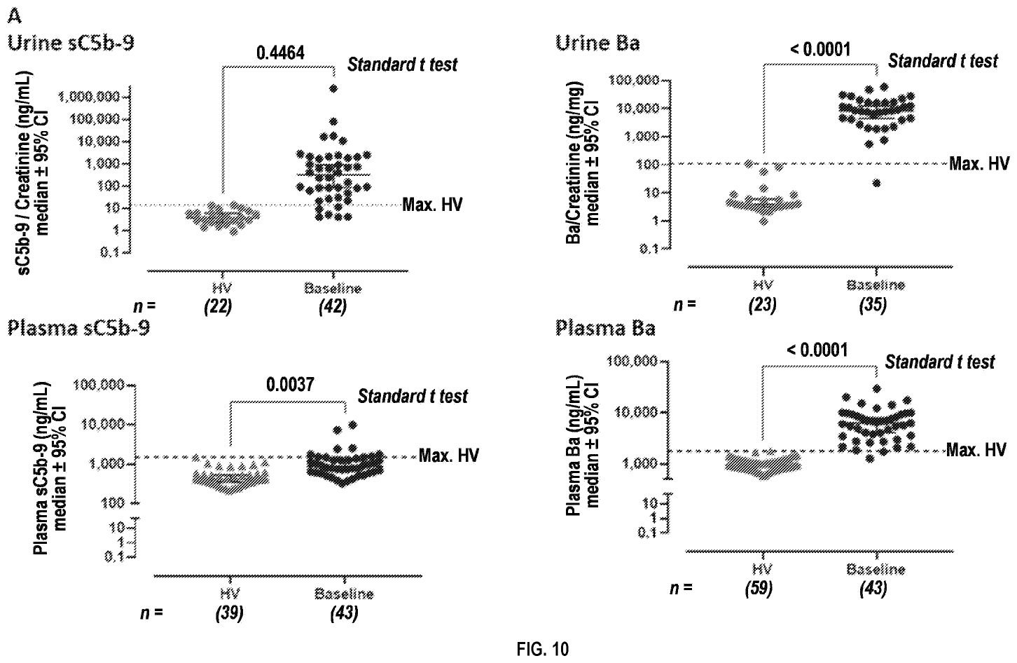

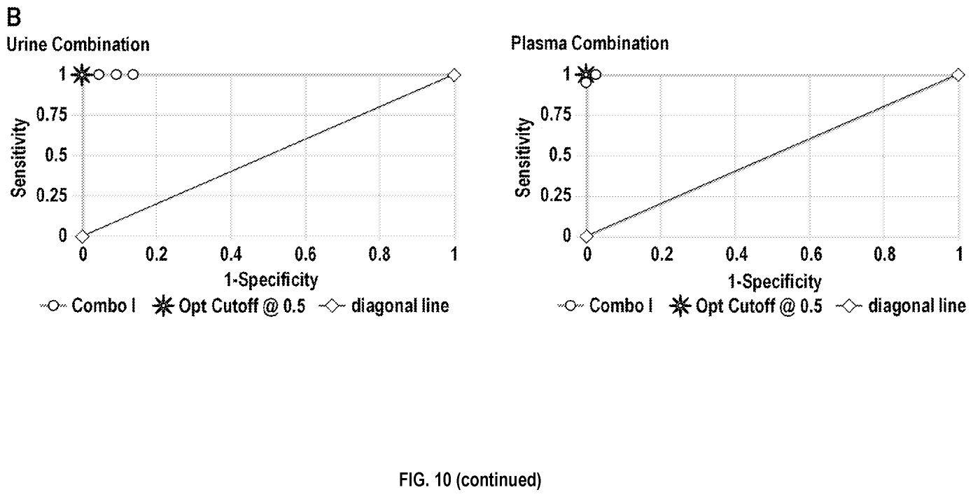

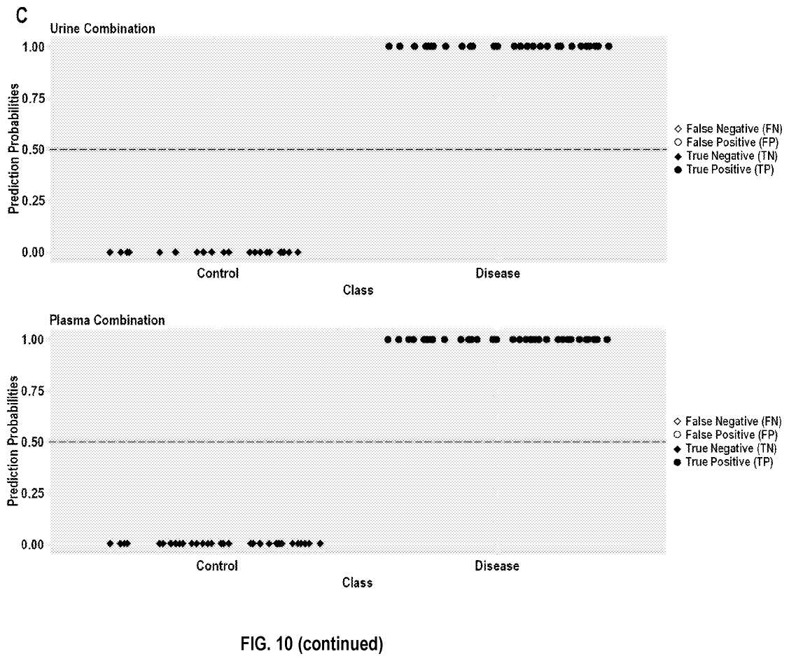

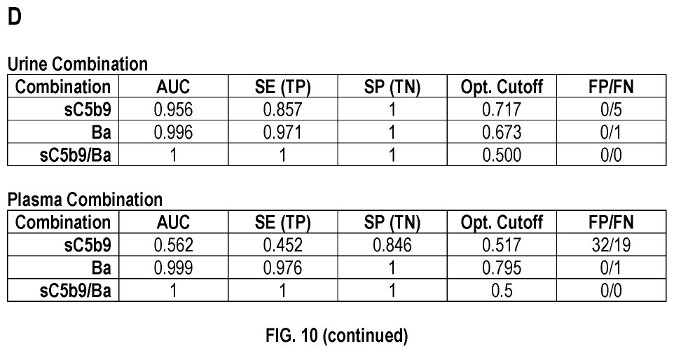

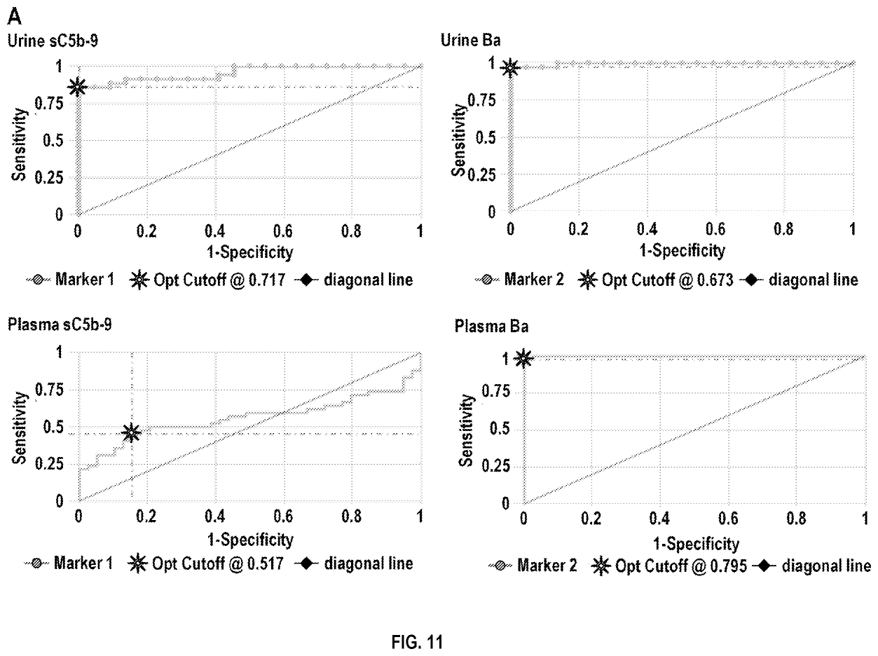

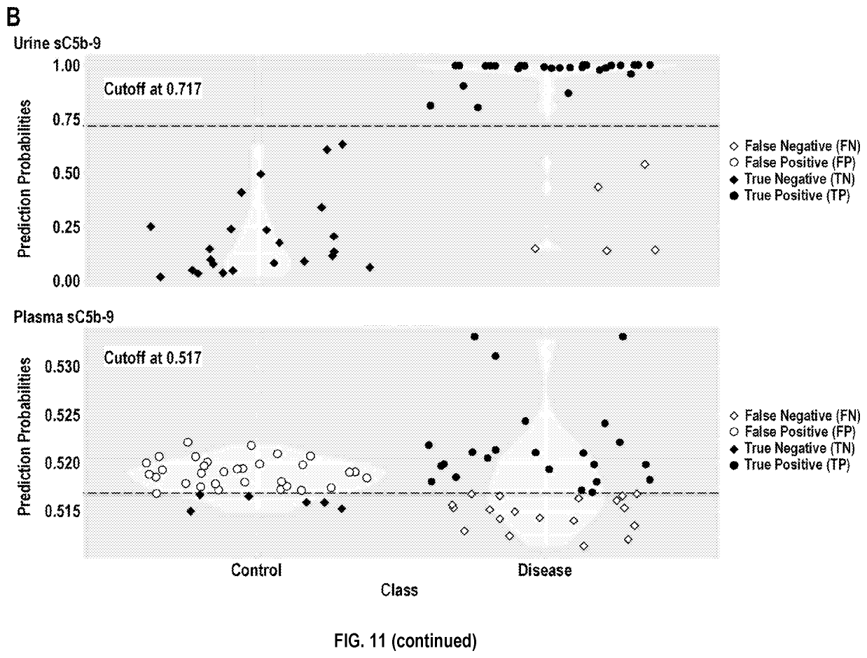

The CombiROC analysis of baseline Ba and sC5b-9 levels, in plasma and urine, in aHUS patients versus normal donor levels, showed that when the biomarkers were analyzed individually some degree of overlap of the two groups was present (FIG. 10A). When a combined biomarker analysis was used, this effect disappeared such that high specificity and selectivity was achieved in both urine and plasma (FIGS. 10B, C and D).

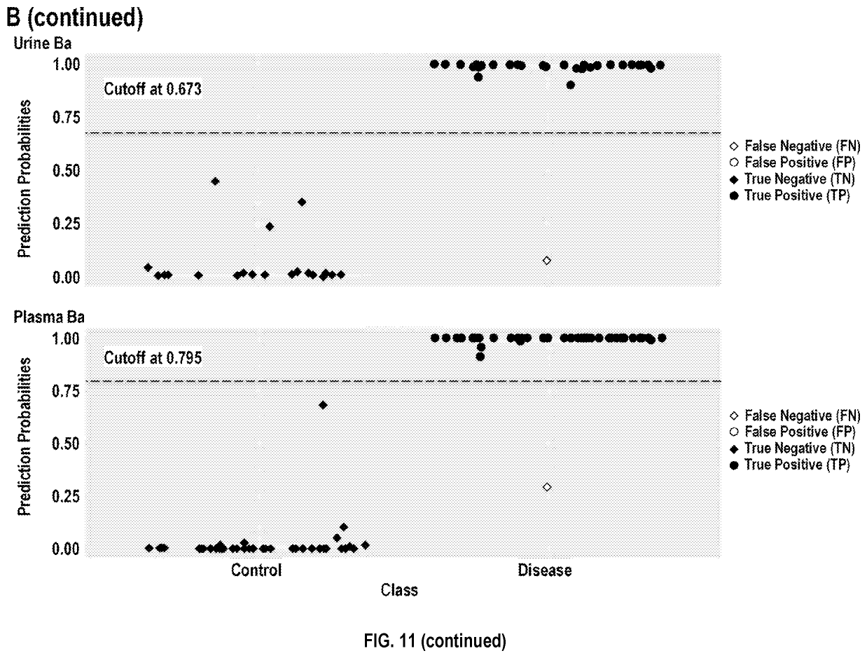

For individual area under the curve values, sC5b-9 was considerably larger in urine than in plasma (0.72 vs 0.52) whereas for Ba it was larger in plasma (0.67 vs 0.80) (FIG. 11A). More false-positive (FP) and false-negative (FN) results occurred in plasma sC5b-9 than urine sC5b-9 (32 FP, 19 FN vs. 0 FP, 5 FN); however, the distributions for Ba levels in urine and plasma were very similar, with few false results (FIG. 11B).

### DISCUSSION

In this study, biomarkers associated with activity of the alternative and terminal complement pathways, namely Ba and sC5b-9 respectively, were found to be significantly associated with baseline PE/PI and dialysis status, alongside baseline clinical measures associated with kidney function, namely eGFR and UPCR. Baseline plasma Ba and urine sC5b-9 levels both showed associations with complete TMA response at 52 weeks. Differences were observed in the utility of these biomarkers based on the analysis medium (plasma vs serum vs urine) and for urine biomarkers, whether they were normalized against creatinine levels. For example, plasma but not urine sC5b-9 and urine but not plasma Ba were associated with patients needing PE/PI within 7 days of treatment initiation. Since a plasma infusion inherently alters blood biomarker levels, urine would be expected to be the more reliable medium for biomarker testing in this setting. However, when normalized against creatinine levels, urinary Ba was no longer significantly associated with PE/PI status. Furthermore, while both urine and plasma Ba were significantly associated with dialysis status within the 5 days prior to treatment initiation, only urine sC5b-9 had this association; urinary Ba, but not sC5b-9, remained significantly associated with dialysis status when normalized against creatinine.

The analysis medium was also relevant to other sC5b-9 associations. Baseline levels of sC5b-9 were found to be elevated over normal donor levels to a much greater extent when analyzed in urine. Additionally, baseline urine but not plasma sC5b-9 was significantly associated with change in UPCR at 52 weeks; and urine but not plasma sC5b-9 levels decreased over 52 weeks of treatment. These observations, along with our ROC analyses of sC5b-9 in urine versus plasma, point to urine being the more reliable analysis medium.

The results of this study suggest that Ba and sC5b-9—particularly when analyzed in urine samples—show potential for use as biomarkers of CM-TMAs, as evidenced by their association with select clinical measures and outcomes, alongside their almost ubiquitous elevation in adults with aHUS compared with the maximum observed levels in normal donors. The reliability of their detection in urine, particularly when in combination, was further evidenced by FIG. 10.

The dot plots (FIG. 10A) graphically depicts the relative distribution of Ba and sC5b-9 levels in aHIUS vs HVs. The AUC curves (FIG. 10B) depicts the combined sensitivity/1-specificity when Ba and sC5b-9 values are combined using CombiROC web-based algorithm, the violin plots (FIG. 10C) provide a visual of the distributions of aHUS and HV relative the Optimal Threshold cutoff for true positive/true negative/false positive/false negative and the summary table (FIG. 10D) provides the actual values from the COmbiROC analyses, which is the tabulated form of the FIG. 10C violin plots. The CombiROC figures in the supplemental section are the individual results. As such, this evidence confirms that combining these 2 complement-specific biomarkers is clinically more sensitive and specific than either one alone at measuring activity and identifying a complement-mediated TMA patient, to predict a clinical benefit from complement inhibitor therapy.

This analysis suggests that these biomarkers may show utility for diagnosis and prognosis of specific facets of CM-TMA disease, particularly those relating to kidney function.

The results of this study support the use of sC5b-9 (particularly urinary sC5b-9) as a potential biomarker of aHUS, with a specific case for its use as a marker of renal (dys)function, as evidenced by its significant association with changes in UPCR during treatment. Further, sC5b-9 levels were also associated with baseline PE/PI requirements (plasma sC5b-9) and dialysis status (urinary sC5b-9).

### CONCLUSION

The results of this analysis highlight a key set of biomarkers with potential diagnostic and prognostic utility in the management of aHUS, particularly in urine. These data also demonstrate the pharmacodynamic effect on levels of biomarkers in response to anti-complement C5 therapy, alongside their utility for predicting treatment response, particularly for Ba and sC5b-9. Further assessment and validation of these biomarkers in a larger patient population is needed to guide patient risk stratification and management decisions.

All references cited in this specification are herein incorporated by reference as though each reference was specifically and individually indicated to be incorporated by reference. The citation of any reference is for its disclosure prior to the filing date and should not be construed as an admission that the present disclosure is not entitled to antedate such reference by virtue of prior invention.

It will be understood that each of the elements described above, or two or more together may also find a useful application in other types of methods differing from the type described above. Without further analysis, the foregoing will so fully reveal the gist of the present disclosure that others can, by applying current knowledge, readily adapt it for various applications without omitting features that, from the standpoint of prior art, fairly constitute essential characteristics of the generic or specific aspects of this disclosure set forth in the appended claims. The foregoing embodiments are presented by way of example only.

1. A method for diagnosing complement-mediated thrombotic microangiopathy (CM-TMA) in a subject comprising:
(a) detecting a level of at least one biomarker selected from the group consisting of cystatin C; cystatin C/creatinine ratio; complement factor Ba; complement factor Ba/creatinine ratio; complement sC5b-9; and/or complement sC5b-9/creatinine ratio in a fluid biological sample obtained from the subject; and
(b) comparing the level of the at least one biomarker to a control, wherein an increase in the level in the fluid biological sample of the subject compared to that of the control is indicative that the subject suffers from, is suspected of suffering from, or is at risk of suffering from CM-TMA.

2. (canceled)

3. A method for evaluating the treatment of CM-TMA in a subject comprising:
(a) diagnosing the patient with CM-TMA according to the method of claim 1,
(b) administering the treatment to the subject with CM-TMA;
(c) after administering the treatment, obtaining a second fluid biological sample from the subject with CM-TMA;
(d) detecting a second level of the at least one biomarker in the second fluid biological sample;
(e) comparing the second level of the at least one biomarker to the control.

4-5. (canceled)

6. The method of claim 1, wherein the fluid biological sample comprises serum, blood, urine, plasma, or a mixture thereof.

7-10. (canceled)

11. The method of claim 1, wherein the at least one biomarker is associated with the subject's estimated glomerular filtration rate (eGFR) and/or urine protein creatinine ratio (UPCR).

12-13. (canceled)

14. The method of claim 1, wherein the biomarker comprises a protein biomarker selected from cystatin C, complement factor Ba, and/or sC5b9 and the detection comprises contacting the biomarker with an agent comprising an antibody or an antigen-binding fragment thereof that binds, with specificity, to the biomarker.

15. (canceled)

16. The method of claim 1, wherein the detecting comprises detecting a biomarker signature comprising the following biomarkers: (a) cystatin C+Ba; (b) cystatin C+sC5b9; (c) cystatin C+cystatin C/creatinine; (d) cystatin C+Ba/creatinine; (e) cystatin C+sC5b9/creatinine; (f) Ba+sC5b9; (g) Ba+cystatin C/creatinine; (h) Ba+Ba/creatinine; (i) Ba+sC5b9/creatinine; (j) sC5b9+cystatin C/creatinine; (k) sC5b9+Ba/creatinine; (l) sC5b9+sC5b9/creatinine; (m) cystatin C/creatinine+Ba/creatinine; (n) cystatin C/creatinine+sC5b9/creatinine; or (o) Ba/creatinine+sC5b9/creatinine.

17. The method of claim 1, wherein the detecting comprises detecting a biomarker signature comprising the following biomarkers: (a) cystatin C+Ba+sC5b9; (b) cystatin C+Ba+cystatin C/creatinine; (c) cystatin C+Ba+Ba/creatinine; (d) cystatin C+Ba+sC5b9/creatinine; (e) cystatin C+sC5b9+cystatin C/creatinine; (f) cystatin C+sC5b9+Ba/creatinine; (g) cystatin C+sC5b9+sC5b9/creatinine; (h) cystatin C+cystatin C/creatinine+Ba/creatinine; (i) cystatin C+cystatin C/creatinine+sC5b9/creatinine; (j) cystatin C+Ba/creatinine+sC5b9/creatinine; (k) Ba+sC5b9+cystatin C/creatinine; (l) Ba+sC5b9+Ba/creatinine; (m) Ba+sC5b9+sC5b9/creatinine; (n) Ba+cystatin C/creatinine+Ba/creatinine; (o) Ba+cystatin C/creatinine+sC5b9/creatinine; (p) Ba+Ba/creatinine+sC5b9/creatinine; (q) sC5b9+cystatin C/creatinine+Ba/creatinine; (r) sC5b9+cystatin C/creatinine+sC5b9/creatinine; (s) sC5b9+Ba/creatinine+sC5b9/creatinine; or (t) cystatin C/creatinine+Ba/creatinine+sC5b9/creatinine.

18. The method of claim 1, wherein the detecting comprises detecting a biomarker signature comprising the following biomarkers: (a) cystatin C+Ba+sC5b9+cystatin C/creatinine; (b) cystatin C+Ba+sC5b9+Ba/creatinine; (c) cystatin C+Ba+sC5b9+sC5b9/creatinine; (d) cystatin C+sC5b9+cystatin C/creatinine+(e) cystatin C+sC5b9+cystatin C/creatinine+sC5b9/creatinine; (f) cystatin C+cystatin C/creatinine+Ba/creatinine+sC5b9/creatinine; (g) Ba+sC5b9+cystatin C/creatinine+Ba/creatinine; (h) Ba+sC5b9+cystatin C/creatinine+sC5b9/creatinine; (i) Ba+cystatin C/creatinine+Ba/creatinine+sC5b9/creatinine; or (j) sC5b9+cystatin C/creatinine+Ba/creatinine+sC5b9/creatinine.

19. The method of claim 1, wherein the detecting comprises detecting a biomarker signature comprising the following biomarkers: (a) cystatin C+Ba+sC5b9+cystatin C/creatinine+Ba/creatinine; (b) cystatin C+Ba+sC5b9+cystatin C/creatinine+sC5b9/creatinine; (c) cystatin C+sC5b9+cystatin C/creatinine+Ba/creatinine+sC5b9/creatinine; or (d) Ba+sC5b9+cystatin C/creatinine+Ba/creatinine+sC5b9/creatinine.

20. The method of claim 1, wherein the detecting comprises detecting a biomarker signature comprising the following biomarkers: (a) cystatin C+Ba+sC5b9+cystatin C/creatinine+Ba/creatinine+sC5b9/creatinine.

21-25. (canceled)

26. The method of claim 1, wherein the fluid biological sample comprises a heterogeneous sample comprising urine and plasma and the detecting comprises detecting a biomarker signature comprising at least one urine biomarker and at least one plasma biomarker selected from: (a) urine cystatin C+plasma Ba; (b) urine cystatin C+plasma sC5b9; (c) urine Ba+plasma Ba; (d) urine Ba+plasma sC5b9; (e) urine sC5b9+plasma Ba; (f) urine sC5b9+plasma sC5b9; (g) urine cystatin C/creatinine+plasma Ba; (h) urine cystatin C/creatinine+plasma sC5b9; (i) urine Ba/creatinine+plasma Ba; (j) Ba/creatinine+plasma sC5b9; (k) urine sC5b9/creatinine+plasma Ba; or (l) urine sC5b9/creatinine+plasma sC5b9.

27. The method of claim 1, wherein the fluid biological sample comprises a heterogeneous sample comprising urine and plasma and the detecting comprises detecting a biomarker signature comprising at least one urine biomarker and at least two plasma biomarker selected from: (a) urine cystatin C+plasma Ba+plasma sC5b9; (b) urine Ba+plasma Ba+plasma sC5b9; (c) urine sC5b9+plasma Ba+plasma sC5b9; (d) urine cystatin C/creatinine+plasma Ba+plasma sC5b9; (e) urine Ba/creatinine+plasma Ba+plasma sC5b9; or (f) urine sC5b9/creatinine+plasma Ba+plasma sC5b9.

28. The method of claim 1, wherein the fluid biological sample comprises a heterogeneous sample comprising urine and plasma and the detecting comprises detecting a biomarker signature comprising at least two urine biomarkers and at least one plasma biomarker selected from: (a) urine cystatin C+urine Ba+plasma Ba OR plasma sC5b9; (b) urine cystatin C+urine sC5b9+plasma Ba OR plasma sC5b9; (c) urine cystatin C+urine cystatin C/creatinine+plasma Ba OR plasma sC5b9; (d) urine cystatin C+urine Ba/creatinine+plasma Ba OR plasma sC5b9; (e) urine cystatin C+urine sC5b9/creatinine+plasma Ba OR plasma sC5b9; (f) urine Ba+urine sC5b9+plasma Ba OR plasma sC5b9; (g) urine Ba+urine cystatin C/creatinine+plasma Ba OR plasma sC5b9; (h) urine Ba+urine Ba/creatinine+plasma Ba OR plasma sC5b9; (i) urine Ba+urine sC5b9/creatinine+plasma Ba OR plasma sC5b9; (j) urine sC5b9+urine cystatin C/creatinine+plasma Ba OR plasma sC5b9; (k) urine sC5b9+urine Ba/creatinine+plasma Ba OR plasma sC5b9; (l) urine sC5b9+urine sC5b9/creatinine+plasma Ba OR plasma sC5b9; (m) urine cystatin C/creatinine+urine Ba/creatinine+plasma Ba OR plasma sC5b9; (n) urine cystatin C/creatinine+urine sC5b9/creatinine+plasma Ba OR plasma sC5b9; or (o) urine Ba/creatinine+urine sC5b9/creatinine+plasma Ba OR plasma sC5b9.

29. The method of claim 1, wherein the at least one biomarker comprises plasma complement factor Ba, sC5b-9, thrombomodulin, and D-dimer; serum sTNF-RI and sVCAM-1; urine factor Ba, sC5b-9, and cystatin C.

30-36. (canceled)

37. The method of claim 1, wherein the subject has been or is being treated with an inhibitor of complement.

38. The method of claim 37, wherein the inhibitor of complement comprises a complement C5 inhibitor selected from the group consisting of an antibody, a small molecule, a polypeptide, a polypeptide analog, a peptidomimetic, and an aptamer, or a combination thereof.

39-53. (canceled)

54. A method for treating complement-mediated thrombotic microangiopathy (CM-TMA) using a complement inhibitor in a manner sufficient to induce a physiological change in CM-TMA-associated biomarker proteins, the method comprising:
(a) determining a level or activity of the CM-TMA-associated biomarker proteins in a biological fluid obtained from the subject, wherein the CM-TMA-associated biomarker proteins are selected from the group consisting of: cystatin C; sVCAM-1, sTNF-R1, thrombomodulin, cystatin C/creatinine ratio; complement factor Ba; complement factor Ba/creatinine ratio; complement sC5b-9; and/or complement sC5b-9/creatinine ratio; and
(b) administering to a subject having, suspected of having, or at risk for developing, CM-TMA, an inhibitor of complement in an amount and with a frequency sufficient to cause a reduction in the levels or activity of the biomarker or the biomarker signature, as compared to the level or activity thereof in a sample of biological fluid of the same type obtained from the subject prior to treatment with the inhibitor.

55-59. (canceled)

60. A kit for the diagnosis of complement-mediated thrombotic microangiopathy (CM-TMA) comprising an assay plate and a binding agent,
wherein the binding agent is an antibody, or an antigen-binding fragment thereof, which, independently, binds specificity to a plurality of biological analytes,
wherein the plurality of biological analytes are protein biomarkers of CM-TMA, and
wherein the protein biomarkers comprise proteolytic fragment of complement component factor B (Ba) and soluble C5b9(sC5b-9).

61. The kit of claim 60, further comprising reagents for creatinine assay.

62. The kit of claim 60, further comprising reagents for determining urine protein/creatinine ratio (UPCR).

63-74. (canceled)
